(19) United States
(12) Patent Application Publication
(10) Pub. No.: US 2025/0249111 A1
(43) Pub. Date: Aug. 7, 2025

(54) ERIBULIN-BASED ANTIBODY-DRUG CONJUGATES AND METHODS OF USE

(71) Applicant: EISAI R&D MANAGEMENT CO., LTD., Tokyo (JP)

(72) Inventors: Sanae YASUDA, Nutley, NJ (US); Lora L. HAMURO, Lawrenceville, NJ (US); Sadhna SHANKAR, Nutley, NJ (US); Yohei OTAKE, Tokyo (JP); Rachael SCOTT, Hatfield Hertfordshire (GB); Calin DUMITRU, Nutley, NJ (US); Seiichi HAYATO, Tokyo (JP)

(21) Appl. No.: 18/856,037

(22) PCT Filed: Apr. 11, 2023
(86) PCT No.: PCT/US2023/018214

§ 371 Date: Oct. 10, 2024

Related U.S. Application Data
(60) Provisional application No. 63/362,882, filed on Apr. 12, 2022.
(60) Provisional application No. 63/366,029, filed on Jun. 8, 2022.

(51) Int. Cl. A61K 47/68; A61K 31/573; A61K 45/06; A61P 35/00; C07K 16/28

(52) U.S. Cl. CPC A61K 47/6803; A61K 31/573; A61K 45/06; A61K 47/6849; A61K 47/6889; A61P 35/00; C07K 16/28

(57) ABSTRACT

Linker toxins and antibody-drug conjugates that bind to human oncology antigen targets such as folate receptor alpha and/or provide anti-tubulin drug activity are disclosed. The linker toxins and antibody-drug conjugates comprise an eribulin drug moiety and can be internalized into target antigen-expressing cells. The disclosure further relates to methods and compositions for use in the treatment of cancer by administering the antibody-drug conjugates provided herein.

### RELATED APPLICATIONS

This application claims the benefit of U.S. Provisional Application Ser. No. 63/362,882, filed Apr. 12, 2022, and 63/366,029, filed Jun. 8, 2022, which are incorporated by reference herein.

### BACKGROUND AND SUMMARY OF THE INVENTION

The present disclosure relates to methods of treating a folate receptor alpha (FRA)-expressing cancer using an antibody drug conjugate (ADC) that binds folate receptor alpha and provides anti-tubulin drug activity. The disclosure further relates to methods of reducing risk of side effects, e.g., interstitial lung disease (ILD), in subjects being treated.

Cancer is among the leading causes of morbidity and mortality worldwide, with approximately 14 million new cases and 8.2 million cancer-related deaths in 2012. The most common causes of cancer death are cancers of: lung (1.59 million deaths); liver (745,000 deaths); stomach (723,000 deaths); colorectal (694,000 deaths); breast (521,000 deaths); and esophagus (400,000 deaths). The number of new cancer cases is expected to rise by about 70% over the next two decades, to approximately 22 million new cancer cases per year (World Cancer Report 2014).

Microtubules are dynamic filamentous cytoskeletal proteins that are involved in a variety of cellular functions, including intracellular migration and transport, cell signaling, and the maintenance of cell shape. Microtubules also play a critical role in mitotic cell division by forming the mitotic spindle required to segregate chromosomes into two daughter cells. The biological functions of microtubules in all cells are regulated in large part by their polymerization dynamics, which occurs by the reversible, non-covalent addition of a and 3 tubulin dimers at both ends of microtubules. This dynamic behavior and resulting control over microtubule length is vital to the proper functioning of the mitotic spindle. Even minor alteration of microtubule dynamics can engage the spindle checkpoint, arrest cell cycle progression at mitosis, and subsequently lead to cell death (Mukhtar et al. (2014) Mol. Cancer Ther. 13:275-84). Due to their rapid cell division, cancer cells are generally more sensitive to compounds that bind to tubulin and disrupt its normal function, as compared to normal cells. For this reason, tubulin inhibitors and other microtubule-targeted agents have become a promising class of drugs for the treatment of cancer (Dumontet and Jordan (2010) Nat. Rev. Drug Discov. 9:790-803).

Folate receptor alpha (FRA) is a glycophosphatidylinositol (GPI)-linked membrane protein that binds folate. While the role of FRA in the biology of normal and cancerous tissue is not fully understood, it is highly over-expressed on a high percentage of ovarian cancers of epithelial origin (O'Shannessy et al. (2013) Int. J. Gynecol. Pathol. 32(3):258-68), as well as in a percentage of non-small cell lung carcinomas (Christoph et al. (2014) Clin. Lung Cancer 15(5):320-30). FRA also has limited expression in normal tissues. These properties make FRA an attractive target for cancer immunotherapy.

The instant inventors have previously demonstrated effective treatment of a folate receptor alpha (FRA)-expressing cancer using compounds, e.g., ADCs, with biological activity against FRA-expressing tumor cells, e.g., an anti-FRA ADC, e.g., MORAb-202. There remains a need, however, for more effective methods and dosing regimens for treating subjects having an FRA-expressing cancer with an anti-FRA ADC, e.g., MORAb-202, e.g., to reduce side effects of treatment.

The present disclosure provides improved methods and dosing regimens for treating a folate receptor alpha (FRA)-expressing cancer using an anti-FRA ADC, e.g., MORAb-202.

In various embodiments, the present disclosure relates to methods of treating a folate receptor alpha (FRA)-expressing cancer, comprising administering to a subject in need thereof an antibody-drug conjugate compound represented by Formula (I):

Ab-(L-D)p (I)
wherein
Ab is an internalizing anti-folate receptor alpha antibody or internalizing antigen-binding fragment thereof comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 4 (LCDR1), SEQ ID NO: 5 (LCDR2), and SEQ ID NO: 6 (LCDR3), as defined by the Kabat numbering system; or three heavy chain complementarity determining regions (HCDRs) comprising amino acid sequences of SEQ ID NO: 7 (HCDR1), SEQ ID NO: 8 (HCDR2), and SEQ ID NO: 9 (HCDR3); and three light chain complementarity determining regions (LCDRs) comprising amino acid sequences of SEQ ID NO: 10 (LCDR1), SEQ ID NO: 11 (LCDR2), and SEQ ID NO: 12 (LCDR3), as defined by the IMGT numbering system;
D is eribulin;
L is a cleavable linker comprising Mal-(PEG)₂-Val-Cit-pAB; and
p is an integer from 1 to 8;
and wherein the antibody-drug conjugate is administered to said subject at a dose of 8 mg to 50 mg of the antibody-drug conjugate per square meter (m²) of the subject's body surface area (BSA).

In various embodiments, the present disclosure relates to methods of reducing risk of interstitial lung disease (ILD) in a subject being treated for an FRA-expressing cancer, comprising administering to the subject an antibody-drug conjugate of Formula (I):

Ab-(L-D)p (I)
wherein
Ab is an internalizing anti-folate receptor alpha antibody or internalizing antigen-binding fragment thereof comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 4 (LCDR1), SEQ ID NO: 5 (LCDR2), and SEQ ID NO: 6 (LCDR3), as defined by the Kabat numbering system; or three heavy chain complementarity determining regions (HCDRs) comprising amino acid sequences of SEQ ID NO: 7 (HCDR1), SEQ ID NO: 8 (HCDR2), and SEQ ID NO: 9 (HCDR3); and three light chain complementarity determining regions (LCDRs) comprising amino acid sequences of SEQ ID NO: 10 (LCDR1), SEQ ID NO: 11 (LCDR2), and SEQ ID NO: 12 (LCDR3), as defined by the IMGT numbering system;
D is eribulin;
L is a cleavable linker comprising Mal-(PEG)₂-Val-Cit-pAB; and
p is an integer from 1 to 8;
and wherein the antibody-drug conjugate is administered to said subject at a dose of 8 mg to 50 mg of the antibody-drug conjugate per square meter (m²) of the subject's body surface area (BSA).

In some embodiments, the antibody or antigen-binding fragment comprises a heavy chain variable region comprising an amino acid sequence of SEQ ID NO: 13, and a light chain variable region comprising an amino acid sequence of SEQ ID NO: 14. In some embodiments, the antibody or antigen-binding fragment comprises a heavy chain comprising an amino acid sequence of SEQ ID NO: 15, and a light chain comprising an amino acid sequence of SEQ ID NO: 16. In some embodiments, the antibody-drug conjugate is MORAb-202.

In some embodiments, p is an integer from 3 to 4.

In some embodiments, the dose of the ADC is 8 mg to 44 mg per m² of the subject's BSA. In some embodiments, the dose of the ADC is 11 mg to 44 mg per m² of the subject's BSA. In some embodiments, the dose is 8 mg to 10 mg per m² of the subject's BSA. In some embodiments, the dose is 33 mg per m² of the subject's BSA. In some embodiments, the dose is 25 mg per m² of the subject's BSA. In some embodiments, the dose is 17 mg per m² of the subject's BSA. In some embodiments, the dose is 15 mg per m² of the subject's BSA. In some embodiments, the dose is 10 mg per m² of the subject's BSA. In some embodiments, the dose is 8 mg per m² of the subject's BSA.

In some embodiments, the ADC is administered to the subject once every three weeks. In some embodiments, the ADC is administered to the subject once every two weeks. In some embodiments, the ADC is administered to the subject once per week.

In some embodiments, the subject has a body weight value that is in the upper quartile for weight.

In some embodiments, the methods disclosed herein reduce a risk of ILD in a subject by at least 5%, at least 10%, at least 15%, at least 16%, at least 17%, at least 18%, at least 19%, or at least 20% following administration of the ADC, as compared to a treatment in which the ADC is administered on a body weight based dose, e.g., at a dose of 0.5 to 2 mg per kilogram of the subject's body weight (BW), e.g., at a dose of 0.9 mg to 1.2 mg per kilogram of BW.

In some embodiments, the methods disclosed herein further comprise administration of a corticosteroid. In some embodiments, the corticosteroid is administered prophylactically. In some embodiments, the corticosteroid is administered concurrently or sequentially with the antibody-drug conjugate. In some embodiments, the corticosteroid is administered before or after the ADC is administered. In some embodiments, the corticosteroid is dexamethasone. In some embodiments, the dexamethasone is administered at a dose of 4 mg dexamethasone. In some embodiments, the dexamethasone is administered at least once a day, or two times a day.

In some embodiments, the dexamethasone is administered for at least three days at the start of treatment with the ADC. In some embodiments, the dexamethasone is administered orally. In some embodiments, the corticosteroid is prednisone. In some embodiments, the prednisone is administered at a dose of at least 0.5 mg, at least 1 mg, or at least 2 mg prednisone. In some embodiments, the prednisone is administered at a dose of 0.5 mg. In some embodiments, the prednisone is administered at a dose of 1 mg. In some embodiments, the prednisone is administered at a dose of 2 mg. In some embodiments, the prednisone is administered at least once a day. In some embodiments, the prednisone is administered for at least 14 days before the start of treatment with the ADC. In some embodiments, the prednisone is administered orally. In some embodiments, the corticosteroid is methylprednisolone. In some embodiments, the methylprednisolone is administered at a dose of 500 to 1000 mg methylprednisolone. In some embodiments, the methylprednisolone is administered at least once a day. In some embodiments, the prednisone is administered for at least three days before the start of treatment with the ADC. In some embodiments, the prednisone is administered intravenously.

In some embodiments, the ADC is administered intravenously.

In some embodiments, a folate receptor alpha-expressing cancer treated by the methods disclosed herein is an ovarian cancer, a breast cancer, a non-small cell lung cancer, or an endometrial cancer. In some embodiments, the ovarian cancer is a platinum resistant ovarian cancer. In some embodiments, the breast cancer is a triple negative breast cancer. In some embodiments, the non-small cell lung cancer is a metastatic non-small cell lung cancer.

In some embodiments, an FRA-expressing cancer treated by the methods disclosed herein is a metastatic cancer. In some embodiments, the metastatic cancer has no genomic alteration. In some embodiments, the metastatic cancer has at least one genomic alteration. In some embodiments, the at least one genomic alteration is in at least one of any of the following genes: EGFR, ALK, PI3K, AKT mTOR, RET, MET, BRAF, NTRK, ROS1, and any gene involved in the RAS-MAPK pathway. In some embodiments, the metastatic cancer is a non-small cell lung cancer (NSCLC). In some embodiments, the FRA-expressing cancer is a refractory cancer. In some embodiments, the refractory cancer is non-responsive to at least one prior treatment, e.g., an approved treatment, e.g., a targeted treatment. As used herein, a “targeted treatment” is a cancer treatment that targets certain genes and/or proteins that are involved in the growth and/or survival of cancer cells. In some embodiments, the targeted treatment is a targeted treatment against any one of the following genes or variants thereof: EGFR, ALK, BRAF, RET, MET, NTRK, and ROS1. In some embodiments, the refractory cancer is non-responsive to an approved treatment, e.g., a platinum-based treatment and/or an immunotherapy-based treatment (e.g., a checkpoint inhibitor therapy). In some embodiments, the refractory cancer is non-responsive to a platinum-based treatment and an immunotherapy-based treatment (e.g., a checkpoint inhibitor therapy), wherein the treatments are administered concurrently or sequentially. In some embodiments, the platinum-based treatment is a platinum-doublet chemotherapy, and the immunotherapy-based treatment is a PD-1 inhibitor or a PD-L1 inhibitor. In various embodiments, the refractory cancer is non-responsive to an anti-CTLA4 inhibitor. In various embodiments, the refractory cancer is non-responsive to radiotherapy. In various embodiments, the refractory cancer is non-responsive to surgery. In various embodiments, the refractory cancer is non-responsive to chemotherapy. In various embodiments, a subject with a refractory cancer is non-responive to not more than 3 prior systemic therapies, e.g., not more than 2 prior systemic therapies. In some embodiments, a subject with a refractory cancer is non-responsive to not more than 1 prior chemotherapy.

In some embodiments, a subject being treated according to the methods disclosed herein does not have one or more of the following: interstitial lung disease (ILD) and/or pneumonitis, a history of ILD and/or pneumonitis, a lung-specific clinically significant illness, pleural effusion, pericardial effusion, prior pneumonectomy, a history of chest radiotherapy within the past 2 years, autoimmune disorder with pulmonary involvement, connective tissue disorder with pulmonary involvement, or inflammatory disorder with pulmonary involvement. In some embodiments, the subject being treated does not have one or more of the following: a history of more than three prior therapies for the FRA-expressing cancer, a high neutrophil-to-lymphocyte ratio, or a serum albumin level at the start of treatment of less than 3 g/dL.

### BRIEF DESCRIPTION OF THE DRAWINGS

FIG. 1 shows the results of the exposure-response (E-R) analysis for ORR in subjects with platinum resistant ovarian cancer (PROC). Subjects were stratified into exposure quartiles. Points represent observed median exposure and ORR per quartile. Vertical bars represent exact 90% confidence intervals. The solid curve in the upper panel represents the logistic regression fit. The shaded band in the upper panel represents the 90% confidence interval of the fit.

FIG. 2 shows the results of the exposure-response (E-R) analysis for ILD in subjects with PROC and across tumor types. Subjects were stratified into exposure quartiles. Points represent observed median exposure and ORR per quartile. Vertical bars represent exact 90% confidence intervals. The solid curve in the upper panel represents the logistic regression fit for a reference subject with a median age of 60 years old. The shaded band in the upper panel represents the 90% confidence interval of the fit.

FIG. 3 shows the simulation results of different dosing regimens of MORAb-202. AIBW=adjusted ideal body weight; BW=body weight; BSA=body surface area. Orange lines in the top panels are LOESS fitted error lines. BW quartiles are indicated by values enclosed by parantheses on the x-axes of the bottom panels.

FIG. 4 shows the predicted median concentrations of MORAb-202 over time with a BW-based dosing regimen and a BSA-based dosing regimen.

FIG. 5 shows the study design using a BSA-based dosing regimen for subjects with ovarian cancer (OC) and/or endometrial cancer (EC).

FIG. 6 shows the study design using a BSA-based dosing regimen for subjects with metastatic non-small cell lung cancer (NSCLC).

FIG. 7 shows a study design using a BSA-based dosing regimen for subjects with platinum resistant high-grade serous ovarian cancer, primary peritoneal cancer, or fallopian tube cancer.

### DETAILED DESCRIPTION

The disclosed methods may be understood more readily by reference to the following detailed description taken in connection with the accompanying figures, which form a part of this disclosure. It is to be understood that the disclosure is not limited to the specific methods described and/or shown herein, and that the terminology used herein is for the purpose of describing particular embodiments by way of example only and is not intended to be limiting of the claimed methods.

Throughout this text, the descriptions refer to methods of using compositions, e.g., an anti-FRA ADC, e.g., MORAb-202. Where the disclosure describes or claims a feature or embodiment associated with a method of using said compositions, such a feature or embodiment is equally applicable to the composition.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control.

It is to be appreciated that certain features of the disclosed methods, which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise.

The terms “about” or “approximately” in the context of numerical values and ranges refers to values or ranges that approximate or are close to the recited values or ranges such that the embodiment may perform as intended, such as having a desired amount of nucleic acids or polypeptides in a reaction mixture, as is apparent to the skilled person from the teachings contained herein. This is due, at least in part, to the varying properties of nucleic acid compositions, age, race, gender, anatomical and physiological variations and the inexactitude of biological systems. Thus, these terms encompass values beyond those resulting from systematic error. It should be understood that this definition of “about” applies to the entirety of the present disclosure, unless otherwise specified in certain contexts, e.g., in descriptions of average number of drug moieties per individual antibody moiety or within a mixture of ADCs.

The term “agent” is used herein to refer to a chemical compound, a mixture of chemical compounds, a biological macromolecule, an extract made from biological materials or a combination thereof. The term “therapeutic agent,” “drug,” or “drug moiety” refers to an agent that is capable of modulating a biological process and/or has biological activity.

The term “antibody” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. The heavy chain of an antibody comprises a heavy chain variable domain (V_{H}) and a heavy chain constant region (C_{H}). The light chain comprises a light chain variable domain (V_{L}) and a light chain constant domain (C_{L}). For the purposes of this application, the mature heavy chain and light chain variable domains each comprises three complementarity determining regions (CDR1, CDR2 and CDR3) within four framework regions (FR1, FR2, FR3 and FR4) arranged from N-terminus to C-terminus: FR1, CDR1, FR2, CDR2, FR3, CDR3 and FR4. An “antibody” can be naturally occurring or man-made, such as monoclonal antibodies produced by conventional hybridoma technology. The term “antibody” includes full-length monoclonal antibodies and full-length polyclonal antibodies, as well as antibody fragments such as Fab, Fab′, F(ab′)2, Fv, and single chain antibodies. An antibody can be any one of the five major classes of immunoglobulins: IgA, IgD, IgE, IgG, and IgM, or subclasses thereof (e.g., isotypes IgG1, IgG2, IgG3, IgG4). The term further encompasses human antibodies, chimeric antibodies, humanized antibodies and any modified immunoglobulin molecule containing an antigen recognition site, so long as it demonstrates the desired biological activity.

The term “chimeric antibody,” as used herein, refers to antibodies wherein the amino acid sequence of the immunoglobulin molecule is derived from two or more species. In some instances, the variable regions of both heavy and light chains corresponds to the variable regions of antibodies derived from one species with the desired specificity, affinity, and activity while the constant regions are homologous to antibodies derived from another species (e.g., human) to minimize an immune response in the latter species.

The term “human antibody,” as used herein, refers an antibody produced by a human or an antibody having an amino acid sequence of an antibody produced by a human.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). Monoclonal antibodies may also be isolated from phage antibody libraries using the techniques described in Clackson et al. (1991) Nature 352:624-8, and Marks et al. (1991) J. Mol. Biol. 222:581-97, for example.

The monoclonal antibodies described herein specifically include “chimeric” antibodies, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity.

The terms “antibody-drug conjugate,” “antibody conjugate,” “conjugate,” “immunoconjugate,” and “ADC” are used interchangeably, and refer to a compound (e.g., eribulin) or derivative thereof that is linked to an antibody (e.g., an anti-FRA antibody) and is defined by the generic formula: Ab-(L-D)ₚ (Formula I), wherein Ab=an antibody moiety (i.e., antibody or antigen-binding fragment), L=a linker moiety, D=a drug moiety, and p=the number of drug moieties per antibody moiety.

The term “antigen-binding fragment” or “antigen-binding portion” of an antibody, as used herein, refers to one or more fragments of an antibody that retain the ability to specifically bind to an antigen (e.g., FRA). Antigen-binding fragments preferably also retain the ability to internalize into an antigen-expressing cell. In some embodiments, antigen-binding fragments also retain immune effector activity. It has been shown that fragments of a full-length antibody can perform the antigen-binding function of a full-length antibody. Examples of binding fragments encompassed within the term “antigen-binding fragment” or “antigen-binding portion” of an antibody include (i) a Fab fragment, a monovalent fragment consisting of the V_{L}, V_{H}, C_{L}, and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody; (v) a dAb fragment, which comprises a single variable domain, e.g., a V_{H} domain (see, e.g., Ward et al. (1989) Nature 341:544-6; and Winter et al., WO 90/05144); and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). See, e.g., Bird et al. (1988) Science 242:423-6; and Huston et al. (1988) Proc. Natl. Acad. Sci. USA 85:5879-83. Such single chain antibodies are also intended to be encompassed within the term “antigen-binding fragment” or “antigen-binding portion” of an antibody, and are known in the art as an exemplary type of binding fragment that can internalize into cells upon binding. See, e.g., Zhu et al. (2010) 9:2131-41; He et al. (2010) J. Nucl. Med. 51:427-32; and Fitting et al. (2015) MAbs 7:390-402. In certain embodiments, scFv molecules may be incorporated into a fusion protein. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-8; and Poljak et al. (1994) Structure 2:1121-3). Antigen-binding fragments are obtained using conventional techniques known to those of skill in the art, and the binding fragments are screened for utility (e.g., binding affinity, internalization) in the same manner as are intact antibodies. Antigen-binding fragments may be prepared by cleavage of the intact protein, e.g., by protease or chemical cleavage.

“Body surface area” or “BSA” as used herein refers to the total surface area of a subject being treated with the methods disclosed herein. A subject's body surface area may be calculated to determine the most appropriate dosage for a compound, e.g., an antibody-drug conjugate, e.g., an anti-FRA ADC, to be administered to the subject. In general, values for a subject's body surface area may be derived from calculations based on the subject's body weight.

The term “cancer” refers to a physiological condition in a mammal in which a population of cells is characterized by unregulated cell growth. Examples of cancers include, but are not limited to, carcinoma, lymphoma, blastoma, sarcoma, leukemia, ovarian cancer (e.g., platinum resistant ovarian cancer), breast cancer (e.g., triple negative breast cancer), non-small cell lung cancer, endometrial cancer, peritoneal cancer, and fallopian tube cancer. Triple negative breast cancer refers to breast cancer that is negative for expression of the genes for estrogen receptor (ER), progesterone receptor (PR), and Her2/neu. More particular examples of such cancers include squamous cell cancer, small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, liver cancer, bladder cancer, hepatoma, osteosarcoma, melanoma, colon cancer, colorectal cancer, uterine cancer, salivary gland carcinoma, kidney cancer, liver cancer, prostate cancer, vulval cancer, thyroid cancer, hepatic carcinoma, peritoneal cancer, fallopian tube cancer, and various types of head and neck cancers.

The terms “cancer cell” and “tumor cell” refer to individual cells or the total population of cells derived from a tumor, including both non-tumorigenic cells and cancer stem cells. As used herein, the term “tumor cell” will be modified by the term “non-tumorigenic” when referring solely to those tumor cells lacking the capacity to renew and differentiate to distinguish those tumor cells from cancer stem cells.

The term “chemotherapeutic agent” or “anti-cancer agent” is used herein to refer to all chemical compounds that are effective in treating cancer regardless of mechanism of action. Inhibition of metastasis or angiogenesis is frequently a property of a chemotherapeutic agent. Non-limiting examples of chemotherapeutic agents include alkylating agents, for example, nitrogen mustards, ethyleneimine compounds, and alkyl sulphonates; antimetabolites, for example, folic acid, purine or pyrimidine antagonists; anti-mitotic agents, for example, anti-tubulin agents such as eribulin or eribulin mesylate (Halaven™) or derivatives thereof, vinca alkaloids, and auristatins; cytotoxic antibiotics; compounds that damage or interfere with DNA expression or replication, for example, DNA minor groove binders; and growth factor receptor antagonists. In addition, chemotherapeutic agents include antibodies, biological molecules, and small molecules. A chemotherapeutic agent may be a cytotoxic or cytostatic agent. The term “cytostatic agent” refers to an agent that inhibits or suppresses cell growth and/or multiplication of cells.

The term “co-administration” or administration “in combination with” one or more therapeutic agents includes concurrent and consecutive administration in any order.

“Corticosteroid,” as used herein, is any compound belonging to a class of steroid hormones that are typically produced in the adrenal cortex of vertebrates. The term as used herein also encompasses synthetic analogs of such hormones, e.g., pharmaceutical compositions which mimic the actions of naturally occurring corticosteroids. Dexamethasone is an exemplary embodiment of a corticosteroid. Prednisone is also an exemplary embodiment of a corticosteroid. Methylprednisolone is another exemplary embodiment of a corticosteroid.

The term “cytotoxic agent” refers to a substance that causes cell death primarily by interfering with a cell's expression activity and/or functioning. Examples of cytotoxic agents include, but are not limited to, anti-mitotic agents, such as eribulin, auristatins (e.g., monomethyl auristatin E (MMAE), monomethyl auristatin F (MMAF)), maytansinoids (e.g., maytansine), dolastatins, duostatins, cryptophycins, vinca alkaloids (e.g., vincristine, vinblastine), taxanes, taxols, and colchicines; anthracyclines (e.g., daunorubicin, doxorubicin, dihydroxyanthracindione); cytotoxic antibiotics (e.g., mitomycins, actinomycins, duocarmycins (e.g., CC-1065), auromycins, duomycins, calicheamicins, endomycins, phenomycins); alkylating agents (e.g., cisplatin); intercalating agents (e.g., ethidium bromide); topoisomerase inhibitors (e.g., etoposide, tenoposide); radioisotopes, such as At²¹¹, I¹³¹, I²⁵, Y⁹⁰, Re¹⁸⁶, Re¹⁸⁸, Sm¹⁵³, Bi²¹² or ²¹³, P³², and radioactive isotopes of lutetium (e.g., Lu¹⁷⁷); and toxins of bacterial, fungal, plant or animal origin (e.g., ricin (e.g., ricin A-chain), diphtheria toxin, Pseudomonas exotoxin A (e.g., PE40), endotoxin, mitogellin, combrestatin, restrictocin, gelonin, alpha-sarcin, abrin (e.g., abrin A-chain), modeccin (e.g., modeccin A-chain), curicin, crotin, Sapaonaria officinalis inhibitor, glucocorticoid).

An “effective amount” of an ADC as disclosed herein is an amount sufficient to perform a specifically stated purpose, for example to produce a therapeutic effect after administration, such as a reduction in tumor growth rate or tumor volume, a reduction in a symptom of cancer, or some other indicia of treatment efficacy. An effective amount can be determined in a routine manner in relation to the stated purpose. The term “therapeutically effective amount” refers to an amount of an ADC effective to treat a disease or disorder in a subject. In the case of cancer, a therapeutically effective amount of ADC can reduce the number of cancer cells, reduce tumor size, inhibit (e.g., slow or stop) tumor metastasis, inhibit (e.g., slow or stop) tumor growth, and/or relieve one or more symptoms. Treatment and therapeutically effective amounts for treatment encompass but do not require complete treatment. For instance, the term encompasses but does not require complete stopping of tumor growth.

The term “epitope” refers to the portion of an antigen capable of being recognized and specifically bound by an antibody. When the antigen is a polypeptide, epitopes can be formed from contiguous amino acids or noncontiguous amino acids juxtaposed by tertiary folding of the polypeptide. The epitope bound by an antibody may be identified using any epitope mapping technique known in the art, including X-ray crystallography for epitope identification by direct visualization of the antigen-antibody complex, as well as monitoring the binding of the antibody to fragments or mutated variations of the antigen, or monitoring solvent accessibility of different parts of the antibody and the antigen. Exemplary strategies used to map antibody epitopes include, but are not limited to, array-based oligo-peptide scanning, limited proteolysis, site-directed mutagenesis, high-throughput mutagenesis mapping, hydrogen-deuterium exchange, and mass spectrometry (see, e.g., Gershoni et al. (2007) 21:145-56; and Hager-Braun and Tomer (2005) Expert Rev. Proteomics 2:745-56).

The term “eribulin,” as used herein, refers to a synthetic analog of halichondrin B, a macrocyclic compound that was originally isolated from the marine sponge Halichondria okadais. The term “eribulin drug moiety” refers to the component of an ADC that has the structure of eribulin, and is attached to the linker of the ADC via its C-35 amine. Eribulin is a microtubule dynamics inhibitor, which is thought to bind tubulin and induce cell cycle arrest at the G2/M phase by inhibiting mitotic spindle assembly. The term “eribulin mesylate” refers to the mesylate salt of eribulin, which is marketed under the trade name Halaven™.

The term “folate receptor alpha” or “FRA,” as used herein, refers to any native form of human FRA. The term encompasses full-length FRA (e.g., NCBI Reference Sequence: NP_000793; SEQ ID NO: 37), as well as any form of human FRA that results from cellular processing. The term also encompasses naturally occurring variants of FRA, including but not limited to splice variants, allelic variants, and isoforms. FRA can be isolated from a human, or may be produced recombinantly or by synthetic methods.

The term “anti-FRA antibody” or “antibody that specifically binds FRA” refers to any form of antibody or fragment thereof that specifically binds FRA, and encompasses monoclonal antibodies (including full length monoclonal antibodies), polyclonal antibodies, and biologically functional antibody fragments so long as they specifically bind FRA. Preferably the anti-FRA antibody used in the ADCs disclosed herein is an internalizing antibody or internalizing antibody fragment. MORAb-003 is an exemplary internalizing anti-human FRA antibody that may be used in the present disclosure, e.g., as part of an anti-FRA ADC. As used herein, the terms “specific,” “specifically binds,” and “binds specifically” refer to the selective binding of the antibody to the target antigen epitope. Antibodies can be tested for specificity of binding by comparing binding to appropriate antigen to binding to irrelevant antigen or antigen mixture under a given set of conditions. If the antibody binds to the appropriate antigen with at least 2, 5, 7, and preferably 10 times more affinity than to irrelevant antigen or antigen mixture, then it is considered to be specific. In one embodiment, a specific anti-FRA antibody is one that only binds the FRA antigen, but does not bind (or exhibits minimal binding) to other antigens.

“Internalizing” as used herein in reference to an antibody or antigen-binding fragment refers to an antibody or antigen-binding fragment that is capable of being taken through the cell's lipid bilayer membrane to an internal compartment (i.e., “internalized”) upon binding to the cell, preferably into a degradative compartment in the cell. For example, an internalizing anti-FRA antibody is one that is capable of being taken into the cell after binding to FRA on the cell membrane.

The term “interstitial lung disease” refers to any one of a group of lung diseases characterized by inflammation which may lead to pulmonary fibrosis. Interstitial lung disease (ILD) is known to be a potential adverse effect associated with immunotherapy treatment. An “adverse effect” or “AE” is any untoward medical occurrence in a subject administered a compound, and does not necessarily implicate a causal relationship with the administered compound or indicate that the compound cannot be used in treatment, but may present a limitation on the compound's use. Methods of identifying and diagnosing interstitial lung disease are well known in the art, for example, evaluating oxygen saturation using pulse oximetry, and assessing ILD-related lung damage using chest computed tomography (CT) scans.

A “linker” or “linker moiety” is any chemical moiety that is capable of covalently joining a compound, usually a drug moiety such as a chemotherapeutic agent, to another moiety such as an antibody moiety. Linkers can be susceptible to or substantially resistant to acid-induced cleavage, peptidase-induced cleavage, light-based cleavage, esterase-induced cleavage, and/or disulfide bond cleavage, at conditions under which the compound or the antibody remains active.

The term “p” or “antibody:drug ratio” or “drug-to-antibody ratio” or “DAR” refers to the number of drug moieties per antibody moiety, i.e., drug loading, or the number of -L-D moieties per antibody or antigen-binding fragment (Ab) in ADCs of Formula I. In compositions comprising multiple copies of ADCs of Formula I, “p” refers to the average number of -L-D moieties per antibody or antigen-binding fragment, also referred to as average drug loading.

“Pharmaceutically acceptable” means approved or approvable by a regulatory agency of the Federal or a state government, or listed in the U.S. Pharmacopeia or other generally recognized pharmacopeia, for use in animals, and more particularly in humans.

A “pharmaceutical composition” refers to a preparation which is in such form as to permit administration and subsequently provide the intended biological activity of the active ingredient(s) and/or to achieve a therapeutic effect, and which contains no additional components which are unacceptably toxic to a subject to which the formulation would be administered. The pharmaceutical composition may be sterile.

A “pharmaceutical excipient” comprises a material such as an adjuvant, a carrier, pH-adjusting and buffering agents, tonicity adjusting agents, wetting agents, preservative, and the like.

By “protein,” as used herein, is meant at least two covalently attached amino acids. The term encompasses polypeptides, oligopeptides, and peptides. In some embodiments, the two or more covalently attached amino acids are attached by a peptide bond. The protein may be made up of naturally occurring amino acids and peptide bonds, for example when the protein is made recombinantly using expression systems and host cells. Alternatively, the protein may include synthetic amino acids (e.g., homophenylalanine, citrulline, ornithine, and norleucine), or peptidomimetic structures, i.e., “peptide or protein analogs,” such as peptoids.

For amino acid sequences, sequence identity and/or similarity may be determined using standard techniques known in the art, including, but not limited to, the local sequence identity algorithm of Smith and Waterman (1981) Adv. Appl. Math. 2:482, the sequence identity alignment algorithm of Needleman and Wunsch (1970) J. Mol. Biol. 48:443, the search for similarity method of Pearson and Lipman (1988) Proc. Nat. Acad. Sci. USA 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (1984) Nucl. Acid Res. 12:387-95, preferably using the default settings, or by inspection. Preferably, percent identity is calculated by FastDB based upon the following parameters: mismatch penalty of 1; gap penalty of 1; gap size penalty of 0.33; and joining penalty of 30 (“Current Methods in Sequence Comparison and Analysis,” Macromolecule Sequencing and Synthesis, Selected Methods and Applications, pp. 127-149 (1988), Alan R. Liss, Inc).

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle (1987) J. Mol. Evol. 35:351-60; the method is similar to that described by Higgins and Sharp (1989) CABIOS 5:151-3. Useful PILEUP parameters including a default gap weight of 3.00, a default gap length weight of 0.10, and weighted end gaps.

Another example of a useful algorithm is the BLAST algorithm, described in: Altschul et al. (1990) J. Mol. Biol. 215:403-10; Altschul et al. (1997) Nucleic Acids Res. 25:3389-402; and Karin et al. (1993) Proc. Natl. Acad. Sci. USA 90:5873-87. A particularly useful BLAST program is the WU-BLAST-2 program which was obtained from Altschul et al. (1996) Methods in Enzymology 266:460-80. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. (1993) Nucl. Acids Res. 25:3389-402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between proteins disclosed herein and variants thereof, including variants of FRA, and variants of antibody variable domains (including individual variant CDRs), are at least 80% to the sequences depicted herein, and more typically with preferably increasing homologies or identities of at least 85%, 90%, 91%, 92%, 93%, 94%, 95%, 96%, 97%, 98%, 99%, and almost 100% or 100%.

In a similar manner, “percent (%) nucleic acid sequence identity” with respect to the nucleic acid sequence of the antibodies and other proteins identified herein is defined as the percentage of nucleotide residues in a candidate sequence that are identical with the nucleotide residues in the coding sequence of the antigen binding protein. A specific method utilizes the BLASTN module of WU-BLAST-2 set to the default parameters, with overlap span and overlap fraction set to 1 and 0.125, respectively.

The terms “subject,” “patient,” and “participant” are used interchangeably herein to refer to any animal, such as any mammal, including but not limited to, humans, non-human primates, rodents, and the like. In some embodiments, the mammal is a mouse. In some embodiments, the mammal is a human.

The terms “tumor” and “neoplasm” refer to any mass of tissue that results from excessive cell growth or proliferation, either benign or malignant, including precancerous lesions.

As used herein, “to treat” or “therapeutic” and grammatically related terms, refer to any improvement of any consequence of disease, such as prolonged survival, less morbidity, and/or a lessening of side effects which are the byproducts of an alternative therapeutic modality. As is readily appreciated in the art, full eradication of disease is a preferred but albeit not a requirement for a treatment act. “Treatment” or “treat,” as used herein, refers to the administration of, e.g., a described ADC, to a subject, e.g., a patient. The treatment can be to cure, heal, alleviate, relieve, alter, remedy, ameliorate, palliate, improve or affect the disorder, the symptoms of the disorder or the predisposition toward the disorder, e.g., a cancer. The terms “treatment” and “therapy” are used interchangeably herein.

In various embodiments, provided herein is a method of reducing risk of interstitial lung disease (ILD) in a subject treated an anti-FRA ADC. “Reducing risk” as used herein refers to a change in incidence rate of ILD (for example, a change in number of subjects with ILD) relative to a comparative treatment. For example, a treatment with an anti-FRA ADC disclosed herein using a BSA-based dosing regimen may reduce the risk of ILD in certain subjects as compared to similar subjects administered the anti-FRA ADC at an equivalent body weight-based dose over a given time period.

The term “upper quartile for weight” as used herein refers to a body weight value that is within the top 25% of all body weight values for a given group of subjects, e.g., a group of adults in a general population or a group of subjects with an FRA-expressing cancer. As used herein, the term is inclusive of the value representing the border of the top 25%. For example, the upper quartile for weight for a given group of subjects can be determined by first arranging the body weight values of the subjects in ascending order, then dividing the set of values into quarters (also known as quartiles), and lastly determining the value between the third and fourth quartiles. A subject who is in the upper quartile for weight has a body weight value that is at least equal to, if not greater than, the value between the third and fourth quartiles. In some embodiments, the value between the third and fourth quartiles for a given group of subjects is near or about 80 kilograms.

### Antibody-Drug Conjugates

The methods of the present disclosure include the use of compounds with anti-cancer activity. In particular, the compounds include an antibody moiety (including an antigen-binding fragment thereof) conjugated (i.e., covalently attached by a linker) to a drug moiety, wherein the drug moiety, e.g., when not conjugated to an antibody moiety, has a cytotoxic or cytostatic effect. In various embodiments, the drug moiety exhibits reduced or no cytotoxicity when bound in a conjugate but resumes cytotoxicity after cleavage from the linker and antibody moiety.

In some embodiments, the ADC comprises a peptide cleavable linker attaching eribulin to an anti-FRA ADC. In some embodiments, the linker comprises a val-cit moiety. In some embodiments, the linker comprises a PEG spacer. In some embodiments, the linker compries a Mal-(PEG)₂-Val-Cit-pAB linker joining eribulin to an anti-FRA antibody (e.g., an anti-FRA antibody such as MORAb-003). In some embodiments, the anti-FRA ADC is MORAb-202. “MORAb-202” refers to an anti-FRA ADC, wherein the anti-FRA antibody or antigen-binding fragment comprises a heavy chain amino acid sequence of SEQ ID NO: 15 and a light chain amino acid sequence of SEQ ID NO: 16, wherein the linker moiety comprises Mal-(PEG)₂-Val-Cit-pAB, and wherein the linker is attached to eribulin via a C-35 amine. In some embodiments, the structure of MORAb-202 (including the sequences of the anti-FRA antibody moiety in the ADC) are disclosed in PCT Application No. PCT/US2017/020529 (published as WO 2017/151979), which is incorporated herein by reference in its entiry.

In some embodiments, the anti-FRA ADC (e.g., MORAb-202) exhibits particularly favorable properties across various categories, such as: (i) the ability to retain one or more therapeutic properties exhibited by the antibody and drug moieties in isolation, (ii) the ability to maintain the specific binding properties of the antibody moiety; (iii) optimal drug loading and drug-to-antibody ratios; (iv) the ability to allow delivery, e.g., intracellular delivery, of the drug moiety via stable attachment to the antibody moiety; (v) the ability to retain ADC stability as an intact conjugate until transport or delivery to a target site; (vi) minimal aggregation of the ADC prior to or after administration; (vii) the ability to allow for the therapeutic effect, e.g., cytotoxic effect, of the drug moiety after cleavage in the cellular environment; (viii) in vivo anti-cancer treatment efficacy comparable to or superior to that of the antibody and drug moieties in isolation; (ix) minimal off-target killing by the drug moiety; and/or (x) desirable pharmacokinetic and pharmacodynamics properties, formulatability, and toxicologic/immunologic profiles.

An ADC compound of the present disclosure may selectively deliver an effective dose of a cytotoxic or cytostatic agent to FRA-expressing cancer cells or to FRA-expressing tumor tissue. It has been discovered that the disclosed ADCs have potent cytotoxic and/or cytostatic activity against cells expressing FRA. Exemplary FRA-expressing cancers include but are not limited to ovarian cancer (e.g., serous ovarian cancer, clear cell ovarian cancer, or platinum resistant ovarian cancer), lung cancer (e.g., non-small cell lung cancer, e.g., metastatic non-small cell lung cancer), breast cancer (e.g., triple negative breast cancer), and endometrial cancer.

An exemplary ADC has Formula I:

Ab-(L-D)hd p (I)

wherein Ab=internalizing anti-folate receptor alpha antibody or internalizing antigen-binding fragment thereof, L=cleavable linker moiety, D=drug moiety, and p=the number of drug moieties per antibody moiety.

### Antibodies

The antibody moiety (Ab) of Formula I includes within its scope any antibody or antigen-binding fragment that specifically binds to FRA on a cancer cell. The antibody or antigen-binding fragment may bind to FRA with a dissociation constant (K_{D}) of ≤1 mM, ≤100 nM or ≤10 nM, or any amount in between, as measured by, e.g., BIAcore® analysis. In certain embodiments, the K_{D} is 1 pM to 500 pM. In some embodiments, the K_{D} is between 500 pM to 1 μM, 1 μM to 100 nM, or 100 mM to 10 nM.

In some embodiments, the antibody moiety is a four-chain antibody (also referred to as an immunoglobulin), comprising two heavy chains and two light chains. In some embodiments the antibody moiety is a two-chain half body (one light chain and one heavy chain), or an antigen-binding fragment of an immunoglobulin.

In some embodiments, the antibody moiety is an internalizing antibody or internalizing antigen-binding fragment thereof. In some embodiments, the internalizing antibody binds to FRA expressed on the surface of a cell and enters the cell upon binding. In some embodiments, the FRA-targeting antibody moiety is MORAb-003. In some embodiments, the drug moiety of the ADC is released from the antibody moiety of the ADC after the ADC enters and is present in a cell expressing the target cancer antigen (i.e., after the ADC has been internalized).

Amino acid and nucleic acid sequences of exemplary antibodies that may be used in the ADCs disclosed herein are set forth in Tables 1-9.

TABLE 1
Antibodies

| mAb | Class/Isotype | Target |
|---|---|---|
| MORAb-003 | humanized | human folate receptor alpha |

TABLE 2
Amino acid sequences of mAb variable regions

| | | | SEQ | |
|---|---|---|---|---|
| | mAb | IgG chain | ID NO | Amino acid sequence |
| 1 | MORAb-003 | Heavy | 13 | EVQLVESGGGVVQPGRSLRLSCSAS |
| | | chain | | GFTFSGYGLSWVRQAPGKGLEWVAM |
| | | | | ISSGGSYTYYADSVKGRFAISRDNA |
| | | | | KNTLFLQMDSLRPEDTGVYFCARHG |
| | | | | DDPAWFAYWGQGTPVTVSS |
| 2 | MORAb-003 | Light | 14 | DIQLTQSPSSLSASVGDRVTITCSV |
| | | chain | | SSSISSNNLHWYQQKPGKAPKPWIY |
| | | | | GTSNLASGVPSRFSGSGSGTDYTFT |
| | | | | ISSLQPEDIATYYCQQWSSYPYMYT |
| | | | | FGQGTKVEIK |

TABLE 3
Nucleic acid sequences encoding mAb
variable regions

| | | IgG | SEQ | |
|---|---|---|---|---|
| | mAb | chain | ID NO | Nucleic acid sequence |
| 1 | MORAb-003 | Heavy | 29 | GAGGTCCAACTGGTGGAGAGCGGTG |
| | | chain | | GAGGTGTTGTGCAACCTGGCCGGTC |
| | | | | CCTGCGCCTGTCCTGCTCCGCATCT |
| | | | | GGCTTCACCTTCAGCGGCTATGGGT |
| | | | | TGTCTTGGGTGAGACAGGCACCTGG |
| | | | | AAAAGGTCTTGAGTGGGTTGCAATG |
| | | | | ATTAGTAGTGGTGGTAGTTATACCT |
| | | | | ACTATGCAGACAGTGTGAAGGGTAG |
| | | | | ATTTGCAATATCGCGAGACAACGCC |
| | | | | AAGAACACATTGTTCCTGCAAATGG |
| | | | | ACAGCCTGAGACCCGAAGACACCGG |
| | | | | GGTCTATTTTTGTGCAAGACATGGG |
| | | | | GACGATCCCGCCTGGTTCGCTTATT |
| | | | | GGGGCCAAGGGACCCCGGTCACCGT |
| | | | | CTCCTCA |
| 2 | MORAb-003 | Light | 30 | GACATCCAGCTGACCCAGAGCCCAA |
| | | chain | | GCAGCCTGAGCGCCAGCGTGGGTGA |
| | | | | CAGAGTGACCATCACCTGTAGTGTC |
| | | | | AGCTCAAGTATAAGTTCCAACAACT |
| | | | | TGCACTGGTACCAGCAGAAGCCAGG |
| | | | | TAAGGCTCCAAAGCCATGGATCTAC |
| | | | | GGCACATCCAACCTGGCTTCTGGTG |
| | | | | TGCCAAGCAGATTCAGCGGTAGCGG |
| | | | | TAGCGGTACCGACTACACCTTCACC |
| | | | | ATCAGCAGCCTCCAGCCAGAGGACA |
| | | | | TCGCCACCTACTACTGCCAACAGTG |
| | | | | GAGTAGTTACCCGTACATGTACACG |
| | | | | TTCGGCCAAGGGACCAAGGTGGAAA |
| | | | | TCAAA |

TABLE 4
Amino acid sequences of mAb Kabat CDRs

| | mAb | IgG chain | SEQ ID NO | Amino acid sequence |
|---|---|---|---|---|
| 1 | MORAb-003 | HC CDR1 | 1 | GYGLS |
| 2 | MORAb-003 | HC CDR2 | 2 | MISSGGSYTYYADSVKG |
| 3 | MORAb-003 | HC CDR3 | 3 | HGDDPAWFAY |
| 4 | MORAb-003 | LC CDR1 | 4 | SVSSSISSNNLH |
| 5 | MORAb-003 | LC CDR2 | 5 | GTSNLAS |
| 6 | MORAb-003 | LC CDR3 | 6 | QQWSSYPYMYT |

TABLE 5
Nucleic acid sequences encoding mAb Kabat CDRs

| | | | SEQ | |
|---|---|---|---|---|
| | | IgG | ID | |
| | mAb | chain | NO | Nucleic acid sequence |
| 1 | MORAb-003 | HC CDR1 | 17 | GGCTATGGGTTGTCT |
| 2 | MORAb-003 | HC CDR2 | 18 | ATGATTAGTAGTGGTGGTAGTTATACCT |
| | | | | ACTATGCAGACAGTGTGAAGGGT |
| 3 | MORAb-003 | HC CDR3 | 19 | CATGGGGACGATCCCGCCTGGTTCGCTT |
| | | | | AT |
| 4 | MORAb-003 | LC CDR1 | 20 | AGTGTCAGCTCAAGTATAAGTTCCAACA |
| | | | | ACTTGCAC |
| 5 | MORAb-003 | LC CDR2 | 21 | GGCACATCCAACCTGGCTTCT |
| 6 | MORAb-003 | LC CDR3 | 22 | CAACAGTGGAGTAGTTACCCGTACATGT |
| | | | | ACACG |

TABLE 6
Amino acid sequences of mAb IMGT CDRs

| | mAb | IgG chain | SEQ ID NO | Amino acid sequence |
|---|---|---|---|---|
| 1 | MORAb-003 | HC CDR1 | 7 | GFTFSGYG |
| 2 | MORAb-003 | HC CDR2 | 8 | ISSGGSYT |
| 3 | MORAb-003 | HC CDR3 | 9 | ARHGDDPAWFAY |
| 4 | MORAb-003 | LC CDR1 | 10 | SSISSNN |
| 5 | MORAb-003 | LC CDR2 | 11 | GTS |
| 6 | MORAb-003 | LC CDR3 | 12 | QQWSSYPYMYT |

TABLE 7
Nucleic acid sequences encoding mAb IMGT CDRs

| | | | SEQ | |
|---|---|---|---|---|
| | | IgG | ID | |
| | mAb | chain | NO | Nucleic acid sequence |
| 1 | MORAb-003 | HC CDR1 | 23 | GGCTTCACCTTCAGCGGCTATGGG |
| 2 | MORAb-003 | HC CDR2 | 24 | ATTAGTAGTGGTGGTAGTTATACC |
| 3 | MORAb-003 | HC CDR3 | 25 | GCAAGACATGGGGACGATCCCGCCTGG |
| | | | | TTCGCTTAT |
| 4 | MORAb-003 | LC CDR1 | 26 | TCAAGTATAAGTTCCAACAAC |
| 5 | MORAb-003 | LC CDR2 | 27 | GGCACATCC |
| 6 | MORAb-003 | LC CDR3 | 28 | CAACAGTGGAGTAGTTACCCGTACATG |
| | | | | TACACG |

TABLE 8
Amino acid sequences of full-length mAb
Ig chains

| | | | SEQ | |
|---|---|---|---|---|
| | | IgG | ID | |
| | mAb | chain | NO | Amino acid sequence |
| 1 | MORAb- | Heavy | 15 | EVQLVESGGGVVQPGRSLRLSCSASGFTF |
| | 003 | chain | | SGYGLSWVRQAPGKGLEWVAMISSGGSYT |
| | | | | YYADSVKGRFAISRDNAKNTLFLQMDSLR |
| | | | | PEDTGVYFCARHGDDPAWFAYWGQGTPVT |
| | | | | VSSASTKGPSVFPLAPSSKSTSGGTAALG |
| | | | | CLVKDYFPEPVTVSWNSGALTSGVHTFPA |
| | | | | VLQSSGLYSLSSVVTVPSSSLGTQTYICN |
| | | | | VNHKPSNTKVDKKVEPKSCDKTHTCPPCP |
| | | | | APELLGGPSVFLFPPKPKDTLMISRTPEV |
| | | | | TCVVVDVSHEDPEVKFNWYVDGVEVHNAK |
| | | | | TKPREEQYNSTYRVVSVLTVLHQDWLNGK |
| | | | | EYKCKVSNKALPAPIEKTISKAKGQPREP |
| | | | | QVYTLPPSRDELTKNQVSLTCLVKGFYPS |
| | | | | DIAVEWESNGQPENNYKTTPPVLDSDGSF |
| | | | | FLYSKLTVDKSRWQQGNVFSCSVMHEALH |
| | | | | NHYTQKSLSLSPGK |
| 2 | MORAb- | Light | 16 | SSNNLHWYQQKPGKAPKPWIYGTSNLASG |
| | 003 | chain | | VPSRFSGSGSGTDYTFTISSLQPEDIATY |
| | | | | YCQQWSSYPYMYTFGQGTKVEIKRTVAAP |
| | | | | SVFIFPPSDEQLKSGTASVVCLLNNFYPR |
| | | | | EAKVQWKVDNALQSGNSQESVTEQDSKDS |
| | | | | TYSLSSTLTLSKADYEKHKVYACEVTHQG |
| | | | | LSSPVTKSFNRGEC |

TABLE 9

| | Nucleic acid sequences encoding full-length mAb Ig chains⁺ | | | |
|---|---|---|---|---|
| | | | SEQ ID | |
| | mAb | IgG chain | NO | Nucleic acid sequence |
| 1 | MORAb-003 | Heavy chain | 31 | GAGGTCCAACTGGTGGAGAGCGGTGGAGGT |
| | | | | GTTGTGCAACCTGGCCGGTCCCTGCGCCTG |
| | | | | TCCTGCTCCGCATCTGGCTTCACCTTCAGC |
| | | | | GGCTATGGGTTGTCTTGGGTGAGACAGGCA |
| | | | | CCTGGAAAAGGTCTTGAGTGGGTTGCAATG |
| | | | | ATTAGTAGTGGTGGTAGTTATACCTACTAT |
| | | | | GCAGACAGTGTGAAGGGTAGATTTGCAATA |
| | | | | TCGCGAGACAACGCCAAGAACACATTGTTC |
| | | | | CTGCAAATGGACAGCCTGAGACCCGAAGAC |
| | | | | ACCGGGGTCTATTTTTGTGCAAGACATGGG |
| | | | | GACGATCCCGCCTGGTTCGCTTATTGGGGC |
| | | | | CAAGGGACCCCGGTCACCGTCTCCTCAGCC |
| | | | | TCCACCAAGGGCCCATCGGTCTTCCCCCTG |
| | | | | GCACCCTCCTCCAAGAGCACCTCTGGGGGC |
| | | | | ACAGCGGCCCTGGGCTGCCTGGTCAAGGAC |
| | | | | TACTTCCCCGAACCGGTGACGGTGTCGTGG |
| | | | | AACTCAGGCGCCCTGACCAGCGGCGTGCAC |
| | | | | ACCTTCCCGGCTGTCCTACAGTCCTCAGGA |
| | | | | CTCTACTCCCTCAGCAGCGTGGTGACCGTG |
| | | | | CCCTCCAGCAGCTTGGGCACCCAGACCTAC |
| | | | | ATCTGCAACGTGAATCACAAGCCCAGCAAC |
| | | | | ACCAAGGTGGACAAGAAAGTTGAGCCCAAA |
| | | | | TCTTGTGACAAAACTCACACATGCCCACCG |
| | | | | TGCCCAGCACCTGAACTCCTGGGGGGACCG |
| | | | | GACACCCTCATGATCTCCCGGACCCCTGAG |
| | | | | GTCACATGCGTGGTGGTGGACGTGAGCCAC |
| | | | | GAAGACCCTGAGGTCAAGTTCAACTGGTAC |
| | | | | GTGGACGGCGTGGAGGTGCATAATGCCAAG |
| | | | | ACAAAGCCGCGGGAGGAGCAGTACAACAGC |
| | | | | ACGTACCGTGTGGTCAGCGTCCTCACCGTC |
| | | | | CTGCACCAGGACTGGCTGAATGGCAAGGAG |
| | | | | TACAAGTGCAAGGTCTCCAACAAAGCCCTC |
| | | | | CCAGCCCCCATCGAGAAAACCATCTCCAAA |
| | | | | GCCAAAGGGCAGCCCCGAGAACCACAGGTG |
| | | | | TACACCCTGCCCCCATCCCGGGATGAGCTG |
| | | | | ACCAAGAACCAGGTCAGCCTGACCTGCCTG |
| | | | | GTCAAAGGCTTCTATCCCAGCGACATCGCC |
| | | | | GTGGAGTGGGAGAGCAATGGGCAGCCGGAG |
| | | | | AACAACTACAAGACCACGCCTCCCGTGCTG |
| | | | | GACTCCGACGGCTCCTTCTTCTTATATTCA |
| | | | | AAGCTCACCGTGGACAAGAGCAGGTGGCAG |
| | | | | CAGGGGAACGTCTTCTCATGCTCCGTGATG |
| | | | | CATGAGGCTCTGCACAACCACTACACGCAG |
| | | | | AAGAGCCTCTCCCTGTCTCCCGGGAAATGA |
| 2 | MORAb-003 | Light chain | 32 | GACATCCAGCTGACCCAGAGCCCAAGCAGC |
| | | | | CTGAGCGCCAGCGTGGGTGACAGAGTGACC |
| | | | | ATCACCTGTAGTGTCAGCTCAAGTATAAGT |
| | | | | TCCAACAACTTGCACTGGTACCAGCAGAAG |
| | | | | CCAGGTAAGGCTCCAAAGCCATGGATCTAC |
| | | | | GGCACATCCAACCTGGCTTCTGGTGTGCCA |
| | | | | AGCAGATTCAGCGGTAGCGGTAGCGGTACC |
| | | | | GACTACACCTTCACCATCAGCAGCCTCCAG |
| | | | | CCAGAGGACATCGCCACCTACTACTGCCAA |
| | | | | CAGTGGAGTAGTTACCCGTACATGTACACG |
| | | | | TTCGGCCAAGGGACCAAGGTGGAAATCAAA |
| | | | | CGAACTGTGGCTGCACCATCTGTCTTCATC |
| | | | | TTCCCGCCATCTGATGAGCAGTTGAAATCT |
| | | | | GGAACTGCCTCTGTTGTGTGCCTGCTGAAT |
| | | | | AACTTCTATCCCAGAGAGGCCAAAGTACAG |
| | | | | TGGAAGGTGGATAACGCCCTCCAATCGGGT |
| | | | | AACTCCCAGGAGAGTGTCACAGAGCAGGAC |
| | | | | AGCAAGGACAGCACCTACAGCCTCAGCAGC |
| | | | | ACCCTGACGCTGAGCAAAGCAGACTACGAG |
| | | | | AAACACAAAGTCTACGCCTGCGAAGTCACC |
| | | | | CATCAGGGCCTGAGCTCGCCCGTCACAAAG |
| | | | | AGCTTCAACAGGGGAGAGTGTTAA |

⁺Nucleic acid sequences listed do not include leader sequences.

In various embodiments, an ADC disclosed herein may comprise the set of MORAb-003 heavy and light chain variable domains listed in the tables above, or a set of six MORAb-003 CDR sequences (Kabat and/or IMGT) from the heavy and light chains listed in the tables above. In some embodiments, the ADC further comprises human heavy and light chain constant domains or fragments thereof. For instance, the ADC may comprise a human IgG heavy chain constant domain (such as an IgG1) and a human kappa or lambda light chain constant domain. In various embodiments, the antibody moiety of the described ADCs comprises a human immunoglobulin G subtype 1 (IgG1) heavy chain constant domain with a human Ig kappa light chain constant domain. In some embodiments, the anti-FRA antibody moiety in the ADC comprises the full heavy and light chain sequences listed in the tables above.

In various embodiments, the anti-FRA antibody or antigen-binding fragment thereof comprises three heavy chain CDRs and three light chain CDRs as follows: heavy chain CDR1 (HCDR1) comprising SEQ ID NO: 1, heavy chain CDR2 (HCDR2) comprising SEQ ID NO: 2, heavy chain CDR3 (HCDR3) comprising SEQ ID NO: 3; light chain CDR1 (LCDR1) comprising SEQ ID NO: 4, light chain CDR2 (LCDR2) comprising SEQ ID NO: 5, and light chain CDR3 (LCDR3) comprising SEQ ID NO: 6, as defined by the Kabat numbering system (Kabat, Sequences of Proteins of Immunological Interest (National Institutes of Health, Bethesda, Md. (1987 and 1991))).

In some embodiments, the anti-FRA antibody or antigen-binding fragment thereof comprises three heavy chain CDRs and three light chain CDRs as follows: heavy chain CDR1 comprising SEQ ID NO: 7, heavy chain CDR2 comprising SEQ ID NO: 8, heavy chain CDR3 comprising SEQ ID NO: 9; light chain CDR1 comprising SEQ ID NO: 10, light chain CDR2 comprising SEQ ID NO: 11, and light chain CDR3 comprising SEQ ID NO: 12, as defined by the IMGT numbering system (International ImMunoGeneTics Information System (IMGT@)).

In various embodiments, the anti-FRA antibody or antigen-binding fragment thereof comprises a heavy chain variable region comprising the amino acid sequence of SEQ ID NO: 13, and a light chain variable region comprising the amino acid sequence of SEQ ID NO: 14. In some embodiments, the anti-FRA antibody or antigen-binding fragment thereof comprises the heavy chain variable region amino acid sequence of SEQ ID NO: 13 and the light chain variable region amino acid sequence of SEQ ID NO: 14, or sequences that are at least 95% identical to the above-mentioned sequences. In some embodiments, the anti-FRA antibody or antigen-binding fragment thereof has a heavy chain variable region amino acid sequence that is at least 96%, at least 97%, at least 98%, or at least 99% identical to SEQ ID NO: 13 and a light chain variable region amino acid sequence that is at least 96%, at least 97%, at least 98%, or at least 99% identical to SEQ ID NO: 14.

In various embodiments, the anti-FRA antibody comprises a human IgG1 heavy chain constant domain with a human Ig kappa light chain constant domain.

In various embodiments, the anti-FRA antibody comprises the heavy chain amino acid sequence of SEQ ID NO: 15 or a sequence that is at least 95% identical to SEQ ID NO: 15, and the light chain amino acid sequence of SEQ ID NO: 16 or a sequence that is at least 95% identical to SEQ ID NO: 16. In particular embodiments, the antibody comprises the heavy chain amino acid sequence of SEQ ID NO: 15 and the light chain amino acid sequence of SEQ ID NO: 16, or sequences that are at least 95% identical to the above-mentioned sequences. In some embodiments, the anti-FRA antibody has a heavy chain amino acid sequence that is at least 96%, at least 97%, at least 98%, or at least 99% identical to SEQ ID NO: 15 and/or a light chain amino acid sequence that is at least 96%, at least 97%, at least 98%, or at least 99% identical to SEQ ID NO: 16. In some embodiments, the anti-FRA antibody comprises the heavy chain amino acid sequence of SEQ ID NO: 15 and the light chain amino acid sequence of SEQ ID NO: 16. In some embodiments, the anti-FRA antibody comprises a heavy chain encoded by the nucleotide sequence of SEQ ID NO: 35 (with the nucleotides encoding the leader sequence), or SEQ ID NO: 31 (without the nucleotides encoding the leader sequence); and a light chain encoded by the nucleotide sequence of SEQ ID NO: 36 (with the nucleotides encoding the leader sequence), or SEQ ID NO: 32 (without the nucleotides encoding the leader sequence). In some embodiments, the heavy chain amino acid sequence lacks the C-terminal lysine. In various embodiments, the anti-FRA antibody has the amino acid sequence of the antibody produced by a cell line deposited under terms in accordance with the Budapest Treaty with the American Type Culture Collection (ATCC, 10801 University Blvd., Manassas, Va. 20110-2209) on Apr. 24, 2006, under the Accession No. PTA-7552, or such sequences lacking the heavy chain C-terminal lysine. In various embodiments, the anti-FRA antibody is MORAb-003 (USAN name: farletuzumab) (Ebel et al. (2007) Cancer Immunity 7:6), or an antigen-binding fragment thereof.

In various embodiments, amino acid substitutions are of single residues. Insertions usually will be on the order of from about 1 to about 20 amino acid residues, although considerably larger insertions may be tolerated as long as anti-FRA biological function is retained. Deletions usually range from about 1 to about 20 amino acid residues, although in some cases deletions may be much larger. Substitutions, deletions, insertions, or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances. Conservative substitutions are generally made in accordance with the following chart depicted as Table 10.

TABLE 10

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In some embodiments, the FRA-targeting antibody moiety is MORAb-003. In some embodiments, FRA-targeting antibody moieties such as MORAb-003 provide particularly improved drug:antibody ratio, tumor targeting, bystander killing, treatment efficacy, and reduced off-target killing. Improved treatment efficacy can be measured in vitro or in vivo, and may include reduced tumor growth rate and/or reduced tumor volume.

### Linkers

In various embodiments, the linker in an anti-FRA ADC is stable extracellularly in a sufficient manner to be therapeutically effective. In some embodiments, the linker is stable outside a cell, such that the ADC remains intact when present in extracellular conditions (e.g., prior to transport or delivery into an FRA-expressing cell). The term “intact,” used in the context of an ADC, means that the antibody moiety remains attached to the drug moiety. As used herein, “stable,” in the context of a linker or ADC comprising a linker, means that no more than 20%, no more than about 15%, no more than about 10%, no more than about 5%, no more than about 3%, or no more than about 1% of the linkers (or any percentage in between) in a sample of ADC are cleaved (or in the case of an overall ADC are otherwise not intact) when the ADC is present in extracellular conditions.

Whether a linker is stable extracellularly can be determined, for example, by including an anti-FRA ADC in plasma for a predetermined time period (e.g., 2, 4, 6, 8, 16, or 24 hours) and then quantifying the amount of free drug moiety present in the plasma. Stability may allow the ADC time to localize to target tumor cells and prevent the premature release of the drug, which could lower the therapeutic index of the ADC by indiscriminately damaging both normal and tumor tissues. In some embodiments, the linker is stable outside of a target cell and releases the drug moiety from the ADC once inside of the cell, such that the drug moiety can bind to its target (e.g., to microtubules). Thus, an effective linker will: (i) maintain the specific binding properties of the antibody moiety; (ii) allow delivery, e.g., intracellular delivery, of the drug moiety via stable attachment to the antibody moiety; (iii) remain stable and intact until the ADC has been transported or delivered to its target site; and (iv) allow for the therapeutic effect, e.g., cytotoxic effect, of the drug moiety after cleavage.

A linker may be “cleavable” or “non-cleavable” (Ducry and Stump, Bioconjugate Chem. (2010) 21:5-13). Cleavable linkers are designed to release the drug when subjected to certain environment factors, e.g., when internalized into the target cell, whereas non-cleavable linkers generally rely on the degradation of the antibody moiety itself.

In some embodiments, the linker is a cleavable linker. A cleavable linker refers to any linker that comprises a cleavable moiety. As used herein, the term “cleavable moiety” refers to any chemical bond that can be cleaved. Suitable cleavable chemical bonds are well known in the art and include, but are not limited to, acid labile bonds, protease/peptidase labile bonds, photolabile bonds, disulfide bonds, and esterase labile bonds. Linkers comprising a cleavable moiety can allow for the release of the drug moiety from the ADC via cleavage at a particular site in the linker. In various embodiments, cleavage of the anti-FRA antibody from the linked toxin activates or increases the activity of the toxin.

In some embodiments, the linker is cleavable by a cleaving agent, e.g., an enzyme, that is present in the intracellular environment (e.g., within a lysosome or endosome or caveolea). The linker can be, e.g., a peptide linker that is cleaved by an intracellular peptidase or protease enzyme, including, but not limited to, a lysosomal or endosomal protease. In some embodiments, the linker is a cleavable peptide linker. As used herein, a cleavable peptide linker refers to any linker that comprises a cleavable peptide moiety. The term “cleavable peptide moiety” refers to any chemical bond linking amino acids (natural or synthetic amino acid derivatives) that can be cleaved by an agent that is present in the intracellular environment. For instance, a linker may comprise a valine-citrulline (Val-Cit) sequence that is cleavable by a peptidase such as cathepsin, e.g., cathepsin B.

In some embodiments, the linker is an enzyme-cleavable linker and a cleavable peptide moiety in the linker is cleavable by the enzyme. In some embodiments, the cleavable peptide moiety is cleavable by cathepsin B. An exemplary dipeptide that may be cleaved by cathepsin B is valine-citrulline (Val-Cit) (Dubowchik et al. (2002) Bioconjugate Chem. 13:855-69).

In some embodiments, the linker or the cleavable peptide moiety in the linker comprises an amino acid unit. In some embodiments, the amino acid unit allows for cleavage of the linker by a protease, thereby facilitating release of the drug moiety from the ADC upon exposure to one or more intracellular proteases, such as one or more lysosomal enzymes (Doronina et al. (2003) Nat. Biotechnol. 21:778-84; Dubowchik and Walker (1999) Pharm. Therapeutics 83:67-123). Exemplary amino acid units include, but are not limited to, dipeptides. Exemplary dipeptides include, but are not limited to, valine-citrulline (Val-Cit). In some embodiments, the amino acid unit in the linker comprises Val-Cit. An amino acid unit may comprise amino acid residues that occur naturally and/or minor amino acids and/or non-naturally occurring amino acid analogs, such as citrulline.

In some embodiments, the linker in an anti-FRA ADC disclosed herein comprises at least one spacer unit joining the antibody moiety to the drug moiety. In some embodiments, the spacer unit joins a cleavage site (e.g., a cleavable peptide moiety) in the linker to the antibody moiety. In some embodiments, the linker comprises one or more polyethylene glycol (PEG) moieties, e.g., 1, 2, 3, 4, 5, or 6 PEG moieties. In some embodiments, the linker comprises 2 PEG moieties.

In some embodiments, the spacer unit in the linker comprises one or more PEG moieties. In some embodiments, the spacer unit comprises -(PEG)ₘ-, and m is 2. In some preferred embodiments, the spacer unit comprises (PEG)₂.

In some embodiments, the spacer unit links the antibody moiety to the drug moiety indirectly. In some embodiments, the spacer unit links the antibody moiety to the drug moiety indirectly through a cleavable peptide moiety and an attachment moiety to join the spacer unit to the antibody moiety, e.g., a maleimide moiety.

The spacer unit, in various embodiments, attaches to an anti-FRA antibody moiety (i.e., an anti-FRA antibody or antigen-binding fragment thereof) via a maleimide moiety (Mal).

A spacer unit that attaches to the antibody or antigen-binding fragment via a Mal is referred to herein as a “Mal-spacer unit.” The term “maleimide moiety,” as used herein, means a compound that contains a maleimide group and that is reactive with a sulfhydryl group, e.g., a sulfhydryl group of a cysteine residue on the antibody moiety. In some embodiments, the Mal-spacer unit is reactive with a cysteine residue on the antibody or antigen-binding fragment. In some embodiments, the Mal-spacer unit is joined to the antibody or antigen-binding fragment via the cysteine residue. In some embodiments, the Mal-spacer unit comprises a (PEG)₂ moiety.

In certain embodiments, the linker comprises the Mal-spacer unit and a cleavable peptide moiety. In some embodiments, the cleavable peptide moiety comprises an amino acid unit. In some embodiments, the amino acid unit comprises Val-Cit. In some embodiments, the linker comprises Mal-(PEG)₂ and Val-Cit.

In some embodiments, the Mal-spacer unit attaches an anti-FRA antibody moiety (i.e., an anti-FRA antibody or antigen-binding fragment thereof) to the cleavable moiety in the linker. In some embodiments, the Mal-spacer unit attaches the antibody or antigen-binding fragment to a cleavable peptide moiety. In some embodiments, the cleavable peptide moiety comprises an amino acid unit. In some embodiments, the linker comprises Mal-spacer unit-amino acid unit. In some embodiments, the Mal-spacer unit comprises a PEG moiety. In some embodiments, the amino acid unit comprises Val-Cit.

In some embodiments, the linker comprises the structure: Mal-spacer unit-Val-Cit. In some embodiments, the linker comprises the structure: Mal-(PEG)₂-Val-Cit. In some embodiments, the linker comprises the structure: Mal-(PEG)₂-Val-Cit-pAB.

In some embodiments, another spacer unit is used to attach the cleavable moiety in the linker to the drug moiety, e.g., eribulin. In some embodiments, the eribulin is attached to the cleavable moiety in the linker by a self-immolative spacer unit. In certain embodiments, the eribulin is attached to the cleavable moiety in the linker by a self-immolative spacer unit, the cleavable moiety comprises Val-Cit, and a further spacer unit comprising (PEG)₂ joins the cleavable moiety to an anti-FRA antibody moiety. In certain embodiments, the eribulin is joined to an anti-FRA antibody via a Mal-spacer unit in the linker joined to a Val-Cit cleavable moiety and a pAB self-immolative spacer unit.

A spacer unit may be “self-immolative” or “non-self-immolative.” A “non-self-immolative” spacer unit is one in which part or all of the spacer unit remains bound to the drug moiety upon cleavage of the linker. Examples of non-self-immolative spacer units include, but are not limited to, a glycine spacer unit and a glycine-glycine spacer unit. Non-self-immolative spacer units may eventually degrade over time but do not readily release a linked native drug entirely under cellular conditions. A “self-immolative” spacer unit allows for release of the native drug moiety under intracellular conditions. A “native drug” is one where no part of the spacer unit or other chemical modification remains after cleavage/degradation of the spacer unit.

Self-immolation chemistry is known in the art and could be readily selected for the disclosed ADCs. In various embodiments, the spacer unit attaching the cleavable moiety in the linker to the drug moiety (e.g., eribulin) is self-immolative, and undergoes self-immolation concurrently with or shortly before/after cleavage of the cleavable moiety under intracellular conditions.

In certain embodiments, the self-immolative spacer unit in the linker comprises a p-aminobenzyl unit. In some embodiments, a p-aminobenzyl alcohol (pABOH) is attached to an amino acid unit or other cleavable moiety in the linker via an amide bond, and a carbamate, methylcarbamate, or carbonate is made between the pABOH and the drug moiety (Hamann et al. (2005) Expert Opin. Ther. Patents 15:1087-103). In some embodiments, the self-immolative spacer unit is or comprises p-aminobenzyloxycarbonyl (pAB). Without being bound by theory, it is thought that the self-immolation of pAB involves a spontaneous 1,6-elimination reaction (Jain et al. (2015) Pharm Res 32:3526-40).

In various embodiments, the structure of the p-aminobenzyloxycarbonyl (pAB) used in the disclosed ADCs is shown below:

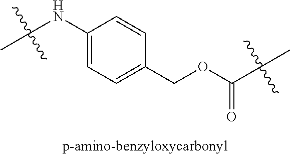 [SMILES: *Nc1ccc(COC(*)=O)cc1]

In various embodiments, the self-immolative spacer unit attaches the cleavable moiety in the linker to the C-35 amine on eribulin. In some embodiments, the self-immolative spacer unit is pAB. In some embodiments, the pAB attaches the cleavable moiety in the linker to the C-35 amine on eribulin. In some embodiments, the pAB undergoes self-immolation upon cleavage of the cleavable moiety, and eribulin is released from the ADC in its native, active form. In some embodiments, an anti-FRA antibody (e.g., MORAb-003) is joined to the C-35 amine of eribulin by a linker comprising Mal-(PEG)₂-Val-Cit-pAB.

In some embodiments, the pAB undergoes self-immolation upon cleavage of a cleavable peptide moiety in the linker. In some embodiments, the cleavable peptide moiety comprises an amino acid unit. In some embodiments, the linker comprises amino acid unit-pAB. In some embodiments, the amino acid unit is Val-Cit. In some embodiments, the linker comprises Val-Cit-pAB (VCP). In various aspects, the antibody moiety of the ADC is conjugated to the drug moiety via a linker, wherein the linker comprises a Mal-spacer unit, a cleavable amino acid unit, and a pAB. In some embodiments, the spacer unit comprises a PEG moiety. In some embodiments, the linker comprises Mal-(PEG)₂-Val-Cit-pAB.

In some embodiments, the antibody moiety is conjugated to the drug moiety via a linker comprising a maleimide moiety (Mal), a polyethylene glycol (PEG) moiety, valine citrulline (Val-Cit or “vc”), and a pAB. In these embodiments, the maleimide moiety covalently attaches the linker-drug moiety to the antibody moiety, and the pAB acts as a self-immolative spacer unit. Such linker may be referred to as the “m-vc-pAB” linker, the “Mal-VCP” linker, the “Mal-(PEG)₂-VCP” linker, or the “Mal-(PEG)₂-Val-Cit-pAB” linker. In some embodiments, the drug moiety is eribulin. The structure of Mal-(PEG)₂-Val-Cit-pAB-eribulin is provided below. The pAB of the Mal-(PEG)₂-Val-Cit-pAB linker is attached to the C-35 amine on eribulin.

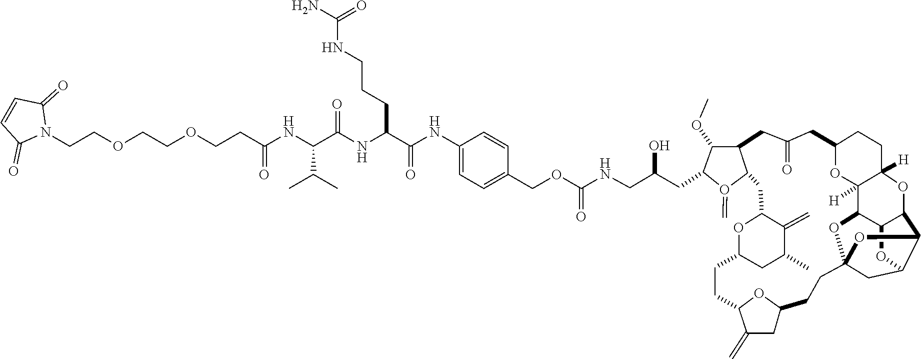

It has been discovered that ADCs comprising Mal-(PEG)₂-Val-Cit-pAB-eribulin demonstrate a particular combination of desirable properties, particularly when paired with an anti-FRA antibody such as MORAb-003 or an antigen-binding fragment thereof. These functional properties are also exemplified in the Examples provided in PCT Application No. PCT/US2017/020529 (published as WO 2017/151979), which is incorporated herein by reference in its entirety.

In some embodiments, an ADC comprises Mal-(PEG)₂-Val-Cit-pAB-eribulin and an antibody moiety comprising an internalizing anti-FRA antibody or an antigen-binding fragment thereof that retains the ability to target and internalize in a tumor cell. In some embodiments, the ADC comprises Mal-(PEG)₂-Val-Cit-pAB-eribulin and an internalizing anti-FRA antibody or internalizing antigen-binding fragment thereof that targets a FRA-expressing tumor cell. In some embodiments, the internalizing antibody or internalizing antigen-binding fragment thereof that targets an FRA-expressing tumor cell comprises three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 4 (LCDR1), SEQ ID NO: 5 (LCDR2), and SEQ ID NO: 6 (LCDR3), as defined by the Kabat numbering system; or three heavy chain complementarity determining regions (HCDRs) comprising amino acid sequences of SEQ ID NO: 7 (HCDR1), SEQ ID NO: 8 (HCDR2), and SEQ ID NO: 9 (HCDR3); and three light chain complementarity determining regions (LCDRs) comprising amino acid sequences of SEQ ID NO: 10 (LCDR1), SEQ ID NO: 11 (LCDR2), and SEQ ID NO: 12 (LCDR3), as defined by the IMGT numbering system. In some embodiments, the internalizing antibody or internalizing antigen-binding fragment thereof that targets an FRA-expressing tumor cell comprises a heavy chain variable region comprising an amino acid sequence of SEQ ID NO: 13, and a light chain variable region comprising an amino acid sequence of SEQ ID NO: 14. In some embodiments, the internalizing antibody or internalizing antigen-binding fragment thereof that targets an FRA-expressing tumor cell comprises a human IgG1 heavy chain constant domain and an Ig kappa light chain constant domain.

In some embodiments, the ADC has Formula I:

Ab-(L-D)ₚ (I)

wherein:
(i) Ab is an internalizing anti-folate receptor alpha (FRA) antibody or internalizing antigen-binding fragment thereof comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 4 (LCDR1), SEQ ID NO: 5 (LCDR2), and SEQ ID NO: 6 (LCDR3), as defined by the Kabat numbering system; or three heavy chain complementarity determining regions (HCDRs) comprising amino acid sequences of SEQ ID NO: 7 (HCDR1), SEQ ID NO: 8 (HCDR2), and SEQ ID NO: 9 (HCDR3); and three light chain complementarity determining regions (LCDRs) comprising amino acid sequences of SEQ ID NO: 10 (LCDR1), SEQ ID NO: 11 (LCDR2), and SEQ ID NO: 12 (LCDR3), as defined by the IMGT numbering system;
(ii) D is eribulin;
(iii) L is a cleavable linker comprising Mal-(PEG)₂-Val-Cit-pAB; and
(iv) p is an integer from 1 to 20.

In some embodiments, the internalizing antibody or internalizing antigen-binding fragment thereof comprises a heavy chain variable region comprising an amino acid sequence of SEQ ID NO: 13, and a light chain variable region comprising an amino acid sequence of SEQ ID NO: 14. In some embodiments, the internalizing antibody is MORAb-003. In some embodiments, p is from 1 to 8, or 1 to 6. In some embodiments, p is from 2 to 8, or 2 to 5. In some embodiments, p is from 3 to 4. In some embodiments, p is 4.

### Drug Moieties

The drug moiety (D) of the ADCs described herein is an anti-tubulin agent, e.g., eribulin.

In some embodiments, the drug moiety is eribulin and the linker of the ADC is attached via the C-35 amine on eribulin.

In various embodiments, the natural form of eribulin used for joining to the linker and antibody moiety is shown below:

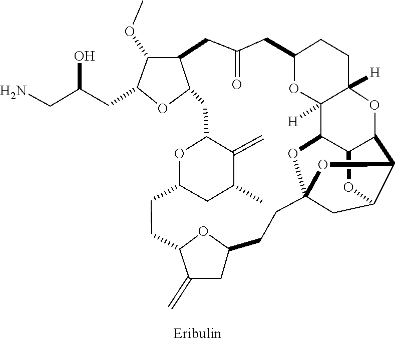

In certain embodiments, an ADC is prepared by reacting an intermediate, which is the precursor of a linker, with eribulin under appropriate conditions. In certain embodiments, reactive groups are used on eribulin and/or the intermediate or linker. The product of the reaction between eribulinand the intermediate is subsequently reacted with an anti-FRA antibody or antigen-binding fragment under appropriate conditions. Alternatively, the linker or intermediate may first be reacted with the antibody or a derivatized antibody, and then reacted with eribulin.

A number of different reactions are available for covalent attachment of eribulin and/or linkers to the antibody moiety. This is often accomplished by reaction of one or more amino acid residues of the antibody molecule, e.g., the sulfhydryl groups of cysteine. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

### Drug Loading

Drug loading is represented by p, and is also referred to herein as the drug-to-antibody ratio (DAR). Drug loading may range from 1 to 20 drug moieties per antibody moiety. In some embodiments, p is an integer from 1 to 20. In some embodiments, p is an integer from 1 to 10, 1 to 9, 1 to 8, 1 to 7, 1 to 6, 1 to 5, 1 to 4, 1 to 3, or 1 to 2. In some embodiments, p is an integer from 2 to 10, 2 to 9, 2 to 8, 2 to 7, 2 to 6, 2 to 5, 2 to 4, or 2 to 3. In some embodiments, p is an integer from 3 to 4. In other embodiments, p is 1, 2, 3, 4, 5, or 6, preferably 3 or 4.

Drug loading may be limited by the number of attachment sites on the antibody moiety. In some embodiments, the linker moiety (L) of the ADC attaches to the antibody moiety through a chemically active group on one or more amino acid residues on the antibody moiety. For example, the linker may be attached to the antibody moiety via a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, or to the sulfhydryl group of one or more cysteine residues). The site to which the linker is attached can be a natural residue in the amino acid sequence of the antibody moiety, or it can be introduced into the antibody moiety, e.g., by DNA recombinant technology (e.g., by introducing a cysteine residue into the amino acid sequence) or by protein biochemistry (e.g., by reduction, pH adjustment, or hydrolysis).

In some embodiments, the number of drug moieties that can be conjugated to an anti-FRA antibody moiety is limited by the number of free cysteine residues. For example, where the attachment is a cysteine thiol group, an anti-FRA antibody may have only one or a few cysteine thiol groups, or may have only one or a few sufficiently reactive thiol groups through which a linker may be attached. Generally, antibodies do not contain many free and reactive cysteine thiol groups that may be linked to a drug moiety. Indeed, most cysteine thiol residues in antibodies exist as disulfide bridges. Over-attachment of linker-toxin to an antibody may destabilize the antibody by reducing the cysteine residues available to form disulfide bridges. Therefore, an optimal drug:antibody ratio should increase potency of the ADC (by increasing the number of attached drug moieties per antibody) without destabilizing the antibody moiety. In some embodiments, an optimal ratio may be about 3-4.

In some embodiments, a linker attached to an antibody moiety through a Mal moiety provides a ratio of about 3-4. In some embodiments, a linker comprising a short spacer unit (e.g., a short PEG spacer unit such as (PEG)₂) provides a ratio of about 3-4. In some embodiments, a linker comprising a peptide cleavable moiety provides a ratio of about 3-4. In some embodiments, an ADC comprising Mal-(PEG)₂-Val-Cit-pAB-eribulin joined to an anti-FRA antibody such as MORAb-003 has a ratio of about 3-4.

In some embodiments, an antibody moiety, e.g., MORAb-003, is exposed to reducing conditions prior to conjugation in order to generate one or more free cysteine residues. In some embodiments, the antibody may be reduced with a reducing agent such as dithiothreitol (DTT) or tris(2-carboxyethyl)phosphine (TCEP), under partial or total reducing conditions, to generate reactive cysteine thiol groups. In certain embodiments, the antibody may be subjected to denaturing conditions to reveal reactive nucleophilic groups on amino acid residues, such as lysine or cysteine.

Where more than one nucleophilic group reacts with a drug-linker intermediate or a linker moiety reagent followed by drug moiety reagent, in a reaction mixture comprising multiple copies of the antibody moiety and linker moiety, then the resulting product may be a mixture of ADC compounds with a distribution of one or more drug moieties attached to each copy of the antibody moiety in the mixture. In some embodiments, the drug loading in a mixture of ADCs resulting from a conjugation reaction ranges from 1 to 20 drug moieties attached per antibody moiety. The average number of drug moieties per antibody moiety (i.e., the average drug loading, or average p) may be calculated by any conventional method known in the art, e.g., by mass spectrometry (e.g., reverse-phase LC-MS), and/or high-performance liquid chromatography (e.g., HIC-HPLC). In some embodiments, the average number of drug moieties per antibody moiety is determined by hydrophobic interaction chromatography-high performance liquid chromatography (HIC-HPLC). In some embodiments, the average number of drug moieties per antibody moiety is determined by reverse-phase liquid chromatography-mass spectrometry (LC-MS). In some embodiments, the average number of drug moieties per antibody moiety is from about 3 to about 4; from about 3.1 to about 3.9; from about 3.2 to about 3.8; from about 3.2 to about 3.7; from about 3.2 to about 3.6; from about 3.3 to about 3.8; or from about 3.3 to about 3.7. In some embodiments, the average number of drug moieties per antibody moiety is from about 3.2 to about 3.8. In some embodiments, the average number of drug moieties per antibody moiety is about 3.8. In some embodiments, the average number of drug moieties per antibody moiety is from 3 to 4; from 3.1 to 3.9; from 3.2 to 3.8; from 3.2 to 3.7; from 3.2 to 3.6; from 3.3 to 3.8; or from 3.3 to 3.7. In some embodiments, the average number of drug moieties per antibody moiety is from 3.2 to 3.8. In some embodiments, the average number of drug moieties per antibody moiety is 3.8.

In some embodiments, the average number of drug moieties per antibody moiety is from about 3.5 to about 4.5; from about 3.6 to about 4.4; from about 3.7 to about 4.3; from about 3.7 to about 4.2; or from about 3.8 to about 4.2. In some embodiments, the average number of drug moieties per antibody moiety is from about 3.6 to about 4.4. In some embodiments, the average number of drug moieties per antibody moiety is about 4.0. In some embodiments, the average number of drug moieties per antibody moiety is from 3.5 to 4.5; from 3.6 to 4.4; from 3.7 to 4.3; from 3.7 to 4.2; or from 3.8 to 4.2. In some embodiments, the average number of drug moieties per antibody moiety is from 3.6 to 4.4. In some embodiments, the average number of drug moieties per antibody moiety is 4.0.

In various embodiments, the term “about” as used with respect to the average number of drug moieties per individual antibody moiety or within a mixture of ADCs, means+/−10%. It should be understood that this definition of “about” applies to all descriptions of average number of drug moieties per individual antibody moiety or within a mixture of ADCs, within the present disclosure.

Individual ADC compounds having particular DAR ratios, or “species,” may be identified in the mixture by mass spectroscopy and separated by UPLC or HPLC, e.g., hydrophobic interaction chromatography (HIC-HPLC). In certain embodiments, a homogeneous or nearly homogenous ADC with a single loading value may be isolated from the conjugation mixture, e.g., by electrophoresis or chromatography.

In some embodiments, drug loading and/or average drug loading in an ADC (e.g., in MORAb-202) is about 4. In some embodiments, a drug loading and/or an average drug loading of about 4 provides beneficial properties. See, e.g., PCT/US2017/020529 (published as WO 2017/151979), which is incorporated herein by reference in its entirety.

In some embodiments, an ADC has Formula I:

Ab-(L-D)ₚ (I)

wherein:
(i) Ab is an internalizing anti-folate receptor alpha antibody or antigen-binding fragment thereof comprising a heavy chain variable region comprising an amino acid sequence of SEQ ID NO: 13, and a light chain variable region comprising an amino acid sequence of SEQ ID NO: 14;
(ii) D is eribulin;
(iii) L is a cleavable linker comprising Mal-(PEG)₂-Val-Cit-pAB; and
(iv) p is an integer from 1 to 8.

In some embodiments, an ADC has Formula I:

Ab-(L-D)ₚ (I)

wherein:
(i) Ab is an internalizing anti-folate receptor alpha antibody or antigen-binding fragment thereof comprising a heavy chain amino acid sequence of SEQ ID NO: 15, and a light chain amino acid sequence of SEQ ID NO: 16;
(ii) D is eribulin;
(iii) L is a cleavable linker comprising Mal-(PEG)₂-Val-Cit-pAB; and
(iv) p is an integer of about 4.

### Therapeutic Uses

In various embodiments, disclosed herein are methods of using the disclosed anti-FRA ADCs, e.g., ADCs comprising the the six CDR amino acid sequences of MORAb-003 attached to a linker comprising Mal-(PEG)₂-Val-Cit-pAB, e.g., MORAb-202, in treating a subject for a disorder, e.g., an oncologic disorder, e.g., a FRA-expressing cancer. ADCs may be administered alone or in conjunction (e.g., simultaneously or sequentially) with a second therapeutic agent (e.g., a corticosteroid, e.g., dexamethasone, prednisone, or methylprednisolone), and may be administered in any pharmaceutically acceptable formulation. In particular, the methods disclosed herein provide uses of the disclosed anti-FRA ADCs for treating a subject having an FRA-expressing cancer, wherein the dosage of the ADC is based on the subject's body surface area (BSA). In some embodiments, methods of treatment employing a BSA-dosing may reduce risk of interstitial lung disease (ILD) in a subject in need of treatment with an anti-FRA ADC. In some embodiments, the methods disclosed herein provide uses of the disclosed anti-FRA ADCs for treating a subject who has previously received at least one systemic anticancer therapy (e.g., cytotoxic or targeted anti-cancer agent). In some embodiments, the methods disclosed herein provide uses of the disclosed anti-FRA ADCs for treating a subject with a metastatic cancer, e.g., a metastatic non-small cell lung cancer. In some embodiments, a subject with a metastatic non-small cell lung cancer treated according to the disclosed methods has no genomic alterations. In some embodiments, a subject with a metastatic FRA-expressing cancer (e.g., a metastatic non-small cell lung cancer) has at least one genomic alteration (e.g., at least one unknown or known genomic alteration). Examples of known genomic alterations include, but are not limited to, genomic alterations in any one of the following genes: EGFR, ALK, PI3K, AKT, mTOR, RET, MET, BRAF, NTRK, ROS1, and any gene involved in the RAS-MAPKpathway. In some embodiments, a subject with a metastatic FRA-expressing cancer has at least one known genomic alteration in at least one of any of the aforementioned genes. As used herein, “genomic alteration” refers to any change to the genome, including, but not limited to, a somatic mutation, a copy number variation, and a gene fusion. In some embodiments, the methods disclosed herein provide uses of the disclosed anti-FRA ADCs for treating a subject with a refractory cancer. As used herein, a “refractory cancer” is a cancer that is non-responsive, i.e., that has not responded, to at least one prior therapy. In some embodiments, a subject with a refractory cancer is non-responsive to a targeted treatment, e.g., a treatment specifically targeted against any one of the following genes or variants thereof: epidermal growth factor receptor (EGFR), anaplastic lymphoma kinase (ALK), v-raf murine sarcoma viral oncogene homolog 1 (BRAF), ret proto-oncogene (RET), MET proto-oncogene, receptor tyrosine kinase (MET), neurotrophic receptor tyrosine kinase (NTRK) and receptor tyrosine kinase (ROS1). As used herein, a “targeted treatment” is a cancer treatment that targets certain genes and/or proteins that are involved in the growth and/or survival of cancer cells. As used herein, the term “variant” is used to refer to any naturally-occurring variant of a gene, including but not limited to splice variants, allelic variants, isoforms, and homologs (e.g., paralogs or orthologs). In contrast to a gene comprising a genomic alteration, a variant of a gene is not linked to or associated with a disease or disorder, e.g., a cancer. In some embodiments, any one of the aforementioned genes (or variants thereof) against which a targeted treatment is specifically targeted may comprise at least one genomic alteration. For example, a targeted treatment may be specific against a mutation in NTRK1, while another targeted treatment may be specific against a mutation in NTRK2.

In some embodiments, a subject with a refractory cancer is non-responsive to a platinum-based treatment (e.g., platinum-doublet chemotherapy) and/or an immunotherapy-based treatment (e.g., an immune checkpoint inhibitor, e.g., a PD-1 inhibitor or a PD-L1 inhibitor). In some embodiments, a subject with a refractory cancer is non-responsive to treatment with platinum-doublet chemotherapy and an immune checkpoint inhibitor (e.g., a PD-1 inhibitor or a PD-L1 inhibitor), wherein the platinum-doublet chemotherapy and immune checkpoint inhibitor are administered concurrently or sequentially. In various embodiments, a subject with a refractory cancer is non-responive to not more than 3 prior systemic therapies, e.g., not more than 2 prior systemic therapies. In some embodiments, a subject with a refractory cancer is non-responsive to not more than 1 prior chemotherapy.

In various embodiments, ADC treatment efficacy may be evaluated for toxicity as well as indicators of efficacy and adjusted accordingly. Efficacy measures include, but are not limited to, objective response rate (ORR). ORR may be measured after a given time period after treatment, e.g., at or after 24 weeks after the start of treatment. ORR may be determined based on tumor assessments according to RECIST, e.g., RECIST 1.1. As used herein, “RECIST” refers to the Response Evaluation Criteria in Solid Tumors (RECIST), a set of standardized guidelines used to measure how well a cancer patient responds to a treatment (Therass et al. (2000) J Natl Cancer Inst. 92:205-16). As used herein, “RECIST 1.1” refers to version 1.1 of RECIST, in which the guidelines are revised and updated relative to an earlier version of RECIST (Eisenhauer et al. (2009) Eur J Cancer. 45:228-47).

In some embodiments, the methods disclosed herein for treating a FRA-expressing cancer comprise administering to a subject in need thereof a therapeutically effective amount of an ADC of Formula (I) as disclosed herein, e.g., MORAb-202, wherein the ADC is administered to the subject at a dose based on the subject's body surface area (BSA).

In some embodiments, the methods disclosed herein for reducing risk of ILD in a subject being treated for an FRA-expressing cancer comprise administering to the subject an ADC of Formula (I) as disclosed herein, e.g., MORAb-202, wherein the ADC is administered to the subject at a dose based on the subject's BSA.

In some embodiments, the ADC is administered at a dose of 8 mg to 50 mg per square meter (m²) of the subject's BSA. In some embodiments, the ADC is administered at a dose of 8 mg to 44 mg per square meter (m²) of the subject's BSA. In some embodiments, the ADC is administered at a dose of 11 mg to 44 mg per square meter (m²) of the subject's BSA. In some embodiments, the ADC is administered at a dose of 8 mg to 10 mg per square meter (m²) of the subject's BSA. In some embodiments, the dose is 5-75, 10-60, 15-45, or 20-40, or 25-35 mg per square meter (m²) of the subject's BSA. In some embodiments, the ADC is administered at a dose of 33 mg per square meter (m²) of the subject's BSA. In some embodiments, the ADC is administered at a dose of 25 mg per square meter (m²) of the subject's BSA. In some embodiments, the ADC is administered at a dose of 17 mg per square meter (m²) of the subject's BSA. In some embodiments, the ADC is administered at a dose of 15 mg per square meter (m²) of the subject's BSA. In some embodiments, the ADC is administered at a dose of 10 mg per square meter (m²) of the subject's BSA. In some embodiments, the ADC is administered at a dose of 8 mg per square meter (m²) of the subject's BSA. Any of these doses may be administered once per week, once every two weeks, or once every three weeks.

BSA may be calculated using any accepted method known in the art. Formulas for calculating a subject's body surface area (BSA) include, for example, the Dubois and Dubois formula (or any variation thereof) (Dubois D, Dubois E F. (1916) Arch Intern Med. 1916; 17:863-871), the Mosteller formula (or any variation thereof) (Mosteller R D. (1987) N Engl J Med. 22; 317(17):1098), or the Haycock formula (Haycock G B et al. (1978) J Pediatr. Jul; 93(1):62-6). Exemplary formulas for calculating BSA are provided below:

BSA (m²)=0.20247×Height (m)^{0.725}×Weight (kg)^{0.425} (DuBois and DuBois); (II)

BSA (m²)=0.007184×Height (cm)^{0.725}×Weight (kg)^{0.425} (variation of Dubois and Dubois); (III)

BSA (m²)=([Height (cm)×Weight (kg)]/3600)^{1/2} (Mosteller); (IV)

or

BSA (m²)=0.024265×Height (cm)^{0.3964}×BW (kg)^{0.5318} (Haycock). (V)

In some embodiments, the actual dose of the ADC to be administered to a subject may be calculated as shown below:

Scheduled dose (mg/m²)×body surface area (BSA) (m²)=Actual dose (mg) (VI)

As used herein, “scheduled dose” refers to a dose selected from the range of doses provided above, e.g., 8 mg to 50 mg, to be administered to a subject. For example, the ADC may be administered at a scheduled dose of 33 mg/m² to a subject with an exemplary BSA of 1.8 square meters (m²), as calculated by Formula III above using hypothetical values of a height of 160 cm and a BW of 80 kg. In this particular example, according to Formula VI provided above, the amount of ADC administered to the subject, or actual dose, is 60.5 mg.

In some embodiments, the treatment dose may be recalculated on the first day of each treatment cycle, using the subject's height measured at intake, and the subject's weight measured on or prior to the first day of each treatment cycle, e.g., two days prior to the first day of each treatment cycle.

In some embodiments, the ADC is administered weekly, every two weeks, every three weeks, monthly, or any time period in between. In some embodiments, the ADC is administered once every three weeks. In some embodiments, the ADC may be administered on a 21-day cycle. A treatment cycle of once every three weeks or a 21-day cycle may also be referred to as “Q3W.” In some embodiments, the ADC is administered once every two weeks.

In some embodiments, the ADC may be administered on a 14-day cycle. A treatment cycle of once every two weeks or a 14-day cycle may also be referred to as “Q2W.” In some embodiments, the ADC is administered once per week. In some embodiments, the ADC may be administered on a 7-day cycle. A treatment cycle of once per week or a 7-day cycle may also be referred to as “QW.”

In some embodiments, the ADC is administered once every three weeks at a BSA-dependent dose of 8 mg/m² to 50 mg/m². In some embodiments, the ADC is administered once every two weeks at a BSA-dependent dose of 8 mg/m² to 50 mg/m². In some embodiments, the ADC is administered once per week at a BSA-dependent dose of 8 mg/m² to 50 mg/m². In some embodiments, the ADC is administered once every three weeks at a BSA-dependent dose of 33 mg/m². In some embodiments, the ADC is administered once every two weeks at a BSA-dependent dose of 33 mg/m². In some embodiments, the ADC is administered once per week at a BSA-dependent dose of 33 mg/m². In some embodiments, the ADC is administered once every three weeks at a BSA-dependent dose of 17 mg/m². In some embodiments, the ADC is administered once every two weeks at a BSA-dependent dose of 17 mg/m². In some embodiments, the ADC is administered once per week at a BSA-dependent dose of 17 mg/m².

In some embodiments, the ADC is administered once every three weeks at a BSA-dependent dose of 15 mg/m². In some embodiments, the ADC is administered once every two weeks at a BSA-dependent dose of 15 mg/m². In some embodiments, the ADC is administered once per week at a BSA-dependent dose of 15 mg/m². In some embodiments, the ADC is administered once every three weeks a BSA-dependent dose of 8 mg/m² to 10 mg/m². In some embodiments, the ADC is administered once every two weeks at a BSA-dependent dose of 8 mg/m² to 10 mg/m². In some embodiments, the ADC is administered once per week at a BSA-dependent dose of 8 mg/m² to 10 mg/m². In some embodiments, the ADC is administered once every three weeks at a BSA-dependent dose of 10 mg/m². In some embodiments, the ADC is administered once every two weeks at a BSA-dependent dose of 10 mg/m². In some embodiments, the ADC is administered once per week at a BSA-dependent dose of 10 mg/m². In some embodiments, the ADC is administered once every three weeks at a BSA-dependent dose of 8 mg/m². In some embodiments, the ADC is administered once every two weeks at a BSA-dependent dose of 8 mg/m². In some embodiments, the ADC is administered once per week at a BSA-dependent dose of 8 mg/m².

Without being bound by theory, BSA-based dosing as disclosed herein may lower exposure levels, e.g., in subjects with higher body weights (BW) when administered the disclosed ADCs, which may help to reduce ILD risk. Without being bound by theory, BW-based dosing may result in higher exposure for subjects in the upper quartile of weight, compared to subjects with a lower body weight. Other exemplary dosing regimens that may reduce the total dose and exposure burden in a treated subject include dosing using BW with a maximum total dose cap or adjusted ideal body weight (AIBW). In some embodiments, BSA-based dosing may be preferred due to its ease-of-use, greater familiarity with practitioners, and reduced likelihood of producing dosing errors.

In some embodiments, a subject treated with an anti-FRA ADC as disclosed herein has a body weight value that is in the upper quartile of weight. In some embodiments, a subject treated with an anti-FRA ADC as disclosed herein has a body weight of at least 80 kg.

In some embodiments, a risk of ILD in a subject treated with an anti-FRA ADC as disclosed herein is reduced by at least 5%, at least 10%, at least 15%, at least 16%, at least 17%, at least 18%, at least 19%, or at least 20% following administration of the ADC at a BSA-based dose, as compared to a treatment in which the ADC is administered on a body weight (BW)-based dose. In some embodiments, the comparative treatment is administered at a dose of 0.5 to 2 mg per kilogram of the subject's body weight (BW), e.g., at a dose of 0.9 mg to 1.2 mg per kilogram of BW. For example, in some embodiments, a comparative treatment may be administered at a dose of 0.9 mg per kilogram of a subject's BW, which is comparable to a dose of 33 mg per square meter of the subject's BSA. In some embodiments, for a subject with an exemplary BW of 80 kg, administration of the ADC at a BW-based dose of 0.9 mg/kg would result in an actual dose of 72 mg; in comparison, administration of the ADC to the same subject at a BSA-based dose of 33 mg/m² would result in an actual dose of 59.4 mg.

In some embodiments, the methods as disclosed herein may further comprise administration of one or more additional therapeutic agents, e.g., one or more additional oncologic agents. In some embodiments, the additional agent comprises a corticosteroid. Without being bound by theory, it is hypothesized that a potential mechanism for ILD may involve an FRA-independent interaction between the anti-FRA ADCs as disclosed herein and pulmonary macrophages in the pre-inflammatory lung microenvironment. An “FRA-independent interaction” refers to an interaction, e.g., binding, between an anti-FRA ADC and a cell that is not caused by recognition and binding to the FRA antigen on the cell. This interaction may result in the induction of cytokines within a subject's lung tissue. Further, without being bound by theory, free eribulin may be released into a subject's lung tissue following the internalization of anti-FRA ADCs into macrophages. Without being bound by theory, the release of free eribulin into the lung tissue may cause tissue damage due to the bystander effects of the ADCs disclosed herein. An immune-mediated mechanism may thus drive the development of ILD. Without being bound by theory, the administration of a corticosteroid may alleviate symptoms or altogether prevent the development of ILD due to its immune modulatory and anti-inflammatory effects.

In some embodiments, a corticosteroid may be administered prophylactically. As used herein, “administered prophylactically” refers to the administration of a treatment, e.g., a corticosteroid, to a subject prior to the subject having or developing symptoms of ILD. In some embodiments, a corticosteroid is administered concurrently or sequentially with an ADC. In some embodiments, a corticosteroid is administered before or after an ADC is administered. In some embodiments, a corticosteroid is dexamethasone. In some embodiments, a corticosteroid is prednisone. In some embodiments, a corticosteroid is methylprednisolone. In some embodiments, the corticosteroid (e.g., dexamethasone, prednisone, or methylprednisolone), may be administered orally or intravenously. In some embodiments, when the corticosteroid (e.g., dexamethasone or prednisone) is administered orally, it may be administered in an amount of 1-10 mg, e.g., 0.5-2 mg, e.g., 2-5 mg, e.g., at 0.5 mg, at 1 mg, at 2 mg, or at 4 mg. In some embodiments, the dexamethasone is administered at a dose determined to be therapeutically effective, e.g., at 4 mg of dexamethasone. In some embodiments, the dexamethasone is administered at least once a day, e.g., two times a day. In some embodiments, the dexamethasone is administered for several days (e.g., 1, 2, 3, 4, 5, or more) before or at the start of treatment with an ADC. In some embodiments, the dexamethasone is administered for at least three days at the start of treatment with an ADC. In some embodiments, the dexamethasone is administered orally. In some embodiments, the prednisone is administered at a dose determined to be therapeutically effective, e.g., at 0.5 mg, at 1 mg, or at 2 mg of prednisone. In some embodiments, the prednisone is administered at 0.5 mg of prednisone. In some embodiments, the prednisone is administered at 1 mg of prednisone. In some embodiments, the prednisone is administered at 2 mg of prednisone. In some embodiments, the prednisone is administered at least once a day. In some embodiments, the prednisone is administered for several days (e.g., 10, 11, 12, 13, 14, or more) before or at the start of treatment with an ADC. In some embodiments, the prednisone is administered for at least 14 days before the start of treatment with an ADC. In some embodiments, the prednisone is administered orally. In some embodiments, when the corticosteroid (e.g., methylprednisolone) is administered intravenously, it may be administered in an amount of 300-1200 mg, e.g., 400-1100 mg, e.g., 500-1000 mg, e.g., at 500 mg, at 750 mg, or at 1000 mg. In some embodiments, the methylprednisolone is administered in an amount of 30-130 mg, e.g., 40-125 mg. In some embodiments, the methylprednisolone is administered at 1 mg per kg of a subject's body weight. In some embodiments, the methylprednisolone is administered at 2 mg per kg of a subject's body weight. In some embodiments, the methylprednisolone is administered intravenously. In some embodiments, the methylprednisolone is administered orally. In some embodiments, when the methylprednisolone is administered orally, it may be administered in an amount of 5-100 mg, e.g., 5-90 mg, e.g., 5-80 mg, e.g., 10-80 mg, e.g., e.g., 10-70 mg, e.g., 10-60 mg. In some embodiments, the methylprednisolone is administered orally at 0.5-1.5 mg per kg of a subject's body weight. In some embodiments, the methylprednisolone is administered orally at 0.5 mg per kg of a subject's body weight. In some embodiments, the methylprednisolone is administered orally at 1 mg per kg of a subject's body weight. In some embodiments, the methylprednisolone is administered orally at 1.5 mg per kg of a subject's body weight. In some embodiments, the methylprednisolone is administered at least once a day. In some embodiments, the methylprednisolone is administered for several days (e.g., 1, 2, 3, 4, 5, or more) before or at the start of treatment with an ADC. In some embodiments, the methylprednisolone is administered for at least three days before the start of treatment with an ADC.

The methods of the present disclosure may be applied to a human subject in need of treatment, e.g., a subject suffering from a cancer, e.g., an FRA-expressing cancer. In some embodiments, the methods disclosed herein may be applied to a non-human mammal having an FRA-expressing cancer for veterinary purposes or as an animal model of human disease. Regarding the latter, such animal models may be useful for evaluating the therapeutic efficacy of the disclosed methods (e.g., testing of dosages and time courses of administration).

The ADCs disclosed herein may be administered to a subject by any suitable administration route to have a therapeutic effect. In some embodiments, the ADC is administered to a subject intravenously.

In some embodiments, the present disclosure features a method of treating a cancer that expresses FRA. The method may be used to treat any human or non-human mammal subject having an FRA-expressing cancer, e.g., those where disruption of tubulin provides a therapeutic benefit. Methods for identifying subjects having cancers that express FRA are known in the art and may be used to identify suitable subjects for treatment with a disclosed ADC. An FRA-expressing cancer may be a primary or metastatic FRA-expressing cancer, or an FRA-expressing cancer which is resistant to a platinum-based therapy, e.g., a platinum resistant cancer. Non-limiting examples of FRA-expressing cancers include gastric cancer, ovarian cancer (e.g., serous ovarian cancer, clear cell ovarian cancer, or platinum resistant ovarian cancer), lung cancer (e.g., non-small cell lung cancer, e.g., metastatic non-small cell lung cancer), lung carcinoid, colorectal cancer, breast cancer (e.g., triple negative breast cancer or hormone receptor (HR)-positive and HER2-low breast cancer), endometrial cancer (e.g., serous endometrial carcinoma), peritoneal cancer (e.g., primary peritoneal cancer), fallopian tube cancer, pancreatic cancer, kidney cancer (e.g., renal cell cancer), cervical cancer, esophageal cancer, and osteosarcoma. In some embodiments, the FRA-expressing cancer is ovarian cancer, e.g., platinum resistant ovarian cancer. In some embodiments, the FRA-expressing cancer is breast cancer, e.g., triple negative breast cancer (TNBC). In some embodiments, the FRA-expressing cancer is non-small cell lung cancer (NSCLC), e.g., metastatic non-small cell lung cancer. In some embodiments, the FRA-expressing cancer is endometrial cancer.

In some embodiments, the present disclosure features a method of reducing risk of ILD in a subject having an FRA-expressing cancer, wherein the subject is in need of treatment. The method may be used with any human or non-human mammal subject having an FRA-expressing cancer for the purpose of reducing ILD risk. An FRA-expressing cancer may be a primary or metastatic FRA-expressing cancer, or an FRA-expressing cancer which is resistant to a platinum-based therapy, e.g., a platinum resistant cancer. Non-limiting examples of FRA-expressing cancers include gastric cancer, ovarian cancer (e.g., serous ovarian cancer, clear cell ovarian cancer, or platinum resistant ovarian cancer), lung cancer (e.g., non-small cell lung cancer, e.g., metastatic non-small cell lung cancer), lung carcinoid, colorectal cancer, breast cancer (e.g., triple negative breast cancer or hormone receptor (HR)-positive and HER2-low breast cancer), endometrial cancer (e.g., serous endometrial carcinoma), peritoneal cancer (e.g., primary peritoneal cancer), fallopian tube cancer, pancreatic cancer, kidney cancer (e.g., renal cell cancer), cervical cancer, esophageal cancer, and osteosarcoma. In some embodiments, the FRA-expressing cancer is ovarian cancer, e.g., platinum resistant ovarian cancer. In some embodiments, the FRA-expressing cancer is breast cancer, e.g., triple negative breast cancer (TNBC). In some embodiments, the FRA-expressing cancer is non-small cell lung cancer (NSCLC), e.g., metastatic non-small cell lung cancer. In some embodiments, the FRA-expressing cancer is endometrial cancer.

In various embodiments, the methods disclosed herein, e.g., the BSA-based dosing using an anti-FRA ADC such as MORAb-202, e.g., at a BSA-dependent dose of 8 to 50 mg/m², are used to treat an ovarian cancer, platinum resistant ovarian cancer, breast cancer, triple negative breast cancer, non-small cell lung cancer, or endometrial cancer. In some embodiments, the methods disclosed herein are used to treat a primary peritoneal cancer or fallopian tube cancer. In some embodiments, a BSA-dependent dose of 33 mg/m² administered once every three weeks (Q3W) is used to treat a platinum resistant ovarian cancer (PROC). In some embodiments, a BSA-dependent dose of 25 mg/m² administered Q3W is used to treat a PROC. In some embodiments, a BSA-dependent dose of 17 mg/m² administered Q3W is used to treat a PROC. In some embodiments, a BSA-dependent dose of 15 mg/m² administered once every two weeks (Q2W) is used to treat a PROC. In some embodiments, a BSA-dependent dose of 8 mg/m² to 10 mg/m² administered once per week (QW) is used to treat a PROC. In some embodiments, the PROC is a serous ovarian cancer. In some embodiments, the PROC is a high-grade serous ovarian cancer. In some embodiments, a BSA-based dose of 33 mg/m² Q3W is used to treat a primary peritoneal cancer. In some embodiments, a BSA-based dose of 25 mg/m² Q3W is used to treat a primary peritoneal cancer. In some embodiments, a BSA-based dose of 17 mg/m² Q3W is used to treat a primary peritoneal cancer. In some embodiments, a BSA-based dose of 15 mg/m² Q2W is used to treat a primary peritoneal cancer. In some embodiments, a BSA-based dose of 8 mg/m² to 10 mg/m² QW is used to treat a primary peritoneal cancer. In some embodiments, a BSA-based dose of 33 mg/m² Q3W is used to treat a fallopian tube cancer. In some embodiments, a BSA-based dose of 25 mg/m² Q3W is used to treat a fallopian tube cancer. In some embodiments, a BSA-based dose of 17 mg/m² Q3W is used to treat a fallopian tube cancer. In some embodiments, a BSA-based dose of 15 mg/m² Q2W is used to treat a fallopian tube cancer. In some embodiments, a BSA-based dose of 8 mg/m² to 10 mg/m² QW is used to treat a fallopian tube cancer. In some embodiments, a subject treated according to the methods disclosed herein has a cancer which recurred within six months of receiving platinum-based therapy.

In some embodiments, a subject who experiences a treatment-related adverse event (TRAE) may be subsequently administered a reduced dose level of MORAb-202. In some embodiments, a subject who is administered a BSA-based dose of 33 mg/m² and experiences a TRAE may be subsequently administered a reduced dose of 25 mg/m². In some embodiments, a subject who is administered a BSA-based dose of 25 mg/m² and experiences a TRAE may be subsequently administered a reduced dose of 17 mg/m². In some embodiments, a subject who is administered a BSA-based dose of 25 mg/m² and experiences a TRAE may be subsequently administered a reduced dose of 15 mg/m². In some embodiments, a subject who is administered a BSA-based dose of 25 mg/m² and experiences a TRAE may be subsequently administered a reduced dose of 8 mg/m² to 10 mg/m². In some embodiments, a subject who is administered a BSA-based dose of 15 mg/m² and experiences a TRAE may be subsequently administered a reduced dose of 8 mg/m² to 10 mg/m². In some embodiments, a TRAE is assessed according to NCI CTCAE v5 and assigned a certain grade level. As used herein, NCI CTCAE v5 refers to version 5 of the National Cancer Institute Common Terminology Criteria for Adverse Events, a descriptive terminology which is utilized for adverse event reporting, wherein a grading (severity) scale is provided for each adverse event term. In some embodiments, a TRAE is an infusion-related reaction of Grade 1 or higher (e.g., Grade 2, 3, or 4). In some embodiments, a TRAE is interstitial lung disease (ILD) or pneumonitis of Grade 1 or higher (e.g., Grade 2, 3, 4). In some embodiments, a TRAE is a decrease in neutrophil count of Grade 3 or 4 or equal to or below 1000 cells/l of the subject's blood sample. In some embodiments, a TRAE is febrile neutropenia of Grade 3 or higher. In some embodiments, a TRAE is a decrease in platelet count of Grade 2 or higher or equal to or below 75,000 cells/μl of the subject's blood sample. In some embodiments, a TRAE is any symptomatic or asymptomatic laboratory result of Grade 3 or higher. In some embodiments, a TRAE is any non-hematologic toxicity of Grade 3 or higher.

In some embodiments, the methods disclosed herein, e.g., the BSA-based dosing using an anti-FRA ADC such as MORAb-202, e.g., at a BSA-dependent dose of 8 to 50 mg/m², reduces the risk of ILD.

Prior to the start of treatment, subjects may be assessed by certain clinical criteria to identify those potentially at higher risk for severe respiratory complications. If a subject is determined to be potentially at higher risk for severe respiratory complications, the subject may be excluded from treatment with the methods disclosed herein. In some embodiments, a subject who is treated according to the foregoing methods is assessed by a pulmonary function test (PFT) prior to treatment. In some embodiments, a subject who is assessed by a PFT and subsequently treated does not have one or more of the following results: a FEV1/FVC ratio of less than 0.7, a FEV1 (forced expiratory volume in the first second) of less than 80%, a FVC (forced vital capacity) of less than 80%, or a DLCO (diffusing capacity of the lungs for carbon monoxide) of less than 80%. In some embodiments, a subject who is treated according to the foregoing methods does not have one or more of the following at the start of treatment: interstitial lung disease (ILD) and/or pneumonitis, a history of ILD and/or pneumonitis, a lung-specific clinically significant illness, pleural effusion, pericardial effusion, prior pneumonectomy, a history of chest radiotherapy within the past 2 years, autoimmune disorder with pulmonary involvement, connective tissue disorder with pulmonary involvement, or inflammatory disorder with pulmonary involvement. Exemplary lung-specific clinically significant illnesses include, but are not limited to, any underlying pulmonary disorder (e.g., pulmonary embolism), asthma, chronic obstructive pulmonary disease (COPD), restrictive lung disease, or any other lung-specific inflammatory disease or condition.

In some embodiments, a subject who is treated according to the foregoing methods does not have one or more of the following: a history of more than three prior therapies for the FRA-expressing cancer, a high neutrophil-to-lymphocyte ratio, or a serum albumin level at the start of treatment of less than 3 g/dL. As used herein, a “high neutrophil-to-lymphocyte ratio” refers to a ratio of neutrophil cells to lymphocyte cells, or “neutrophil-to-lymphocyte ratio” (NLR), in a subject's blood sample that is higher relative to an average NLR of a comparator population, e.g., a group of adults in a general population or a group of subjects with an FRA-expressing cancer. In some embodiments, a high NLR may be at least 3, at least 4, at least 5, at least 6, at least 7, at least 8, or at least 9. NLR may be calculated using any method known in the art. For example, NLR can be calculated by dividing the number of neutrophils by the number of lymphocytes. The number of neutrophils and number of lymphocytes may be measured from a subject's peripheral blood sample. NLR may be calculated using absolute cell counts of neutrophils and/or lymphocytes, or using relative percentages of neutrophils and/or lymphocytes.

### Pharmaceutical Compositions and Formulations

An ADC used in the practice of the foregoing methods may be formulated into a pharmaceutical composition suitable for administration to a subject, e.g., a human subject. In some embodiments, the pharmaceutical composition comprises the ADC and a pharmaceutically acceptable carrier suitable for the desired delivery method. Suitable carriers include any material that, when combined with an ADC disclosed herein, allows that ADC to retain its anti-tumor function and is generally non-reactive with the subject's immune system. Pharmaceutically acceptable carriers may include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In some embodiments the formulation includes one or more isotonic agents, for example, sugars, polyalcohols such as mannitol, sorbitol, or sodium chloride. Pharmaceutically acceptable carriers may comprise minor amounts of auxiliary substances such as wetting or emulsifying agents, preservatives or buffers, which enhance the shelf life or effectiveness of the ADC.

The pharmaceutical compositions described herein may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The preferred form depends on the intended mode of administration and therapeutic application.

Pharmaceutical compositions may be solubilized and administered via any route capable of delivering the composition to the tumor site. Potentially effective routes of administration include, but are not limited to, intravenous, parenteral, intraperitoneal, intramuscular, intratumor, intradermal, intraorgan, orthotopic, and the like. Pharmaceutical compositions can be lyophilized and stored as sterile powders, preferably under vacuum, and then reconstituted in bacteriostatic water (containing for example, benzyl alcohol preservative) or in sterile water prior to injection. Administration can be either systemic or local. Pharmaceutical compositions may comprise an ADC or a pharmaceutically acceptable salt thereof, e.g., a mesylate salt. Pharmaceutical compositions may further comprise a corticosteroid, e.g., dexamethasone, prednisone, or methylprednisolone. Alternatively, in some embodiments, a corticosteroid may be provided in a separate packaging.

In various embodiments, kits for use in the therapeutic applications described herein are within the scope of the present disclosure. Such kits may comprise an ADC disclosed herein and a carrier, package, or container. The carrier, package, or container may be compartmentalized to receive one or more containers such as vials, tubes, and the like, each of the container(s) comprising one of the separate elements to be used in a method disclosed herein, and/or a label or insert comprising instructions for use, such as a use described herein. The carrier, packaging or container may also comprise a compartment for a corticosteroid.

Kits may further comprise one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label may be present on or with the container to indicate that the composition is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that the composition is used for diagnosing or treating a condition, such as a cancer a described herein.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### Example 1

1.1 Dose-Response Relationship for ORR in Subjects with PROC

MORAb-202 was administered to subjects (N=58) with platinum resistant ovarian cancer (PROC) over a body weight (BW)-based dose range of 0.3 to 1.2 mg/kg. All doses were administered at Q3W. A dose-response assessment was carried out to calculate a relationship between dose and objective response rate (ORR) over this dose range.

### 1.2 Dose-Response Relationship for ILD Across Tumor Types

MORAb-202 was administered to subjects (N=82) with PROC, endometrial cancer (EC), triple negative breast cancer (TNBC), and non-small cell lung cancer (NSCLC) over a body weight (BW)-based dose range of 0.3 to 1.2 mg/kg. All doses were administered at Q3W. A dose-response assessment was carried out to calculate a relationship between dose and ILD rate over this dose range.

### 1.2.1 Results

Clinical pharmacology assessments were conducted to evaluate the dose relationships between MORAb-202 and objective response rates (ORR) or ILD. The dose-response relationships were calculated using logistic regression analyses. The results of these analyses are presented in Tables 11 and 12 below.

TABLE 11
Dose-response for ORR in PROC

| BW-based | 0.3 mg/kg, | 0.45 mg/kg, | 0.68 mg/kg, | 0.9 mg/kg, | 1.2 mg/kg, |
|---|---|---|---|---|---|
| dose | N = 1 | N = 3 | N = 2 | N = 28 | N = 24 |
| ORR, N (%) | 0 (0) | 0 (0) | 1 (50) | 9 (32.1) | 13 (54.2) |

TABLE 12
Dose-response for ILD across tumor types

| BW-based | 0.3 mg/kg, | 0.45 mg/kg, | 0.68 mg/kg, | 0.9 mg/kg, | 1.2 mg/kg, |
|---|---|---|---|---|---|
| dose | N = 3 | N = 3 | N = 6 | N = 46 | N = 24 |
| ILD, N (%) | 0 (0) | 0 (0) | 1 (16.7) | 9 (37.0) | 13 (58.3) |

A dose-response relationship was found between MORAb-202 and ORR over the dose range of 0.68 to 1.2 mg/kg in subjects with PROC. Similarly, a dose-response relationship was found between MORAb-202 and ILD over the dose range of 0.68 to 1.2 mg/kg in subjects across tumor types (PROC, EC, TNBC, NSCLC). The highest dose of 1.2 mg/kg had the highest ORR in subjects with PROC and the highest ILD rate in subjects across tumor types. The next lower dose of 0.9 mg/kg corresponded to a lower ORR and ILD rate in both subject groups. Given that the dose of 0.9 mg/kg had a lower ILD rate while still providing a therapeutically meaningful benefit (i.e., ORR>30%), it was selected as the starting dose for subsequent simulation experiments.

1.2.2 Exposure-Response (E-R) Analysis for ORR in Subjects with PROC

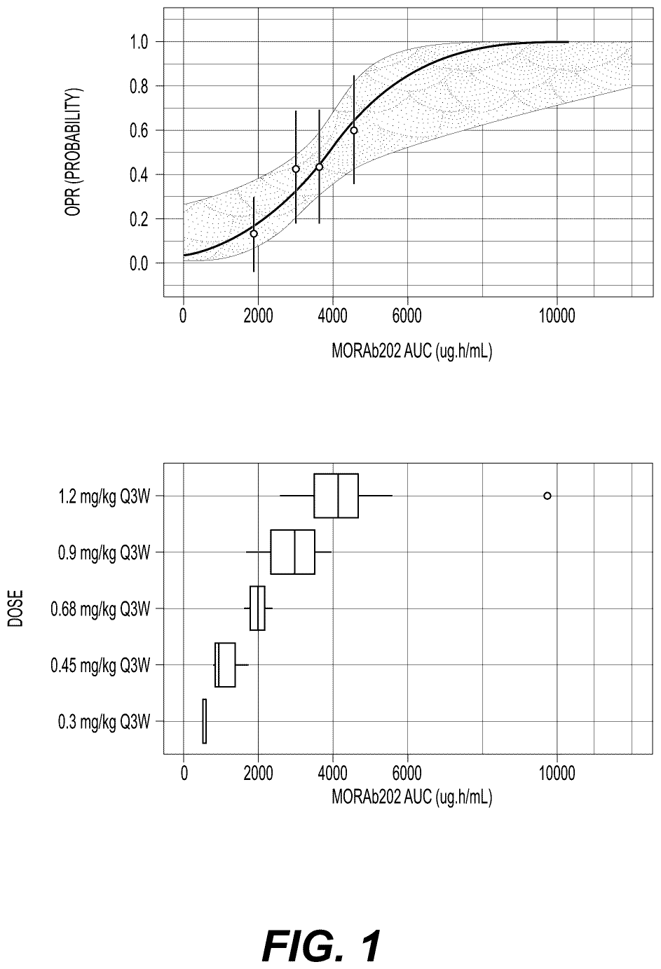

An E-R efficacy (ORR) analysis was conducted with PROC subjects (N=58) across the dose range of 0.3 to 1.2 mg/kg Q3W. Twenty-one subjects had a PR (partial response) and 2 subjects had a CR (complete response) in PROC with an ORR of 39.7% (23/58) across the dose range. Exposure (AUC) was the only significant predictor of the probability of an objective response in multivariate analysis (as shown in FIG. 1). Age, weight, non-high grade serous OC (versus high grade serous OC), ECOG-PS (1 vs 0), and expansion (versus dose escalation) were not significant predictors of an objective response (OR). Higher doses above 1.2 mg/kg Q3W would be expected to result in a higher probability of an OR, given linear PK and lack of saturation of OR across the current dose range. Based on the results of the E-R analysis, clinically meaningful efficacy is expected across the dose range of 0.68 to 1.2 mg/kg. However, as described in section 1.2.3, an overlapping AUC dependence to ILD is observed.

1.2.3 Exposure-Response (E-R) Analysis for ILD in Subjects with PROC and Across all Tumor Types

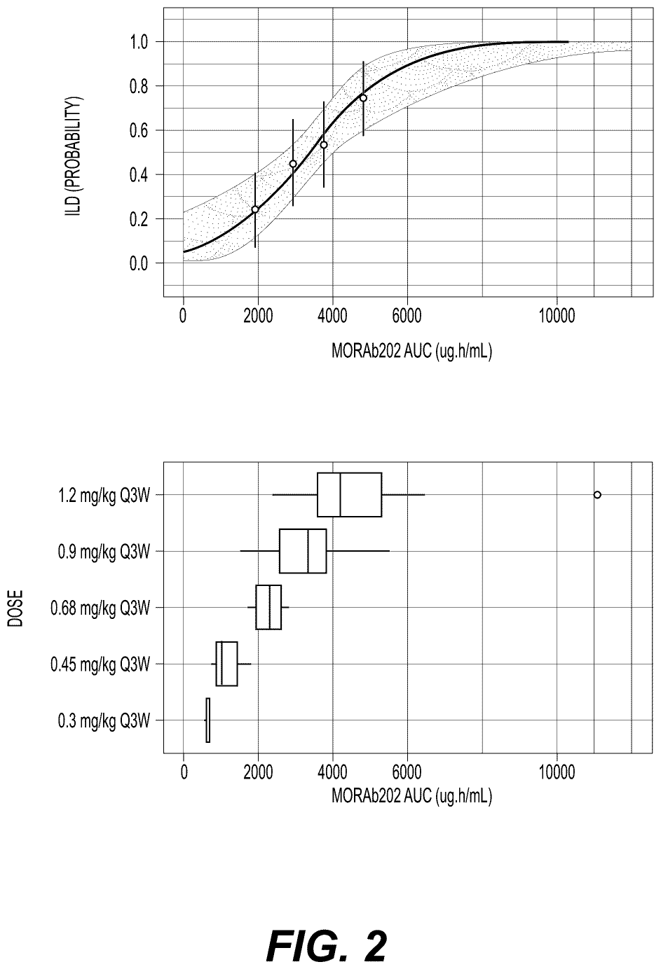

An E-R analysis of ILD was conducted with data from subjects with PROC across the dose range of 0.3 to 1.2 mg/kg Q3W as well as subjects across tumor types (PROC, EC, TNBC, and NSCLC) across the dose range of 0.9 to 1.2 mg/kg Q3W (N=96 in total). Forty-eight subjects had ILD identified by expert review for an ILD rate of 50% (48/96) across both subject groups and dose ranges. AUC and age were significant predictors in multivariate analysis with both higher AUC and higher age predicting a higher probability of ILD. Weight, albumin, ECOG-PS (1 vs 0), Study, and tumor type (OC vs other) were not significant predictors. The probability of ILD by AUC was predicted at the median age (60 years) and plotted along with the observed ILD rate for each exposure quartile (as shown in FIG. 2).

### 1.3 Simulation of Dosing Regimens

Different dosing regimens were simulated to select a dose of MORAb-202 that would minimize ILD rate while maintaining a high ORR. Stochastic simulations were conducted using the MORAb-202 population pharmacokinetics (PPK) model shown in Table 13 to predict AUC and Cₘₐₓ for different dosing regimens. MORAb-202 exposures were found to be dose proportional, and the PK was described by a 2-compartment model with zero-order IV infusion and first-order elimination. BW and serum albumin were significant covariates on CL (total clearance) and BW on volume of distribution. Five dosing regimens were simulated: 1) flat dose of 63 mg; 2) BW-based dose of 0.9 mg/kg; 3) BW-based dose of 0.9 mg/kg with a maximum total dose cap of 70 mg; 4) adjusted ideal body weight (AIBW)-based dose of 1.0 mg/kg; and 5) BSA-based dose of 33 mg/m² (equivalent to BW-based dose of 0.9 mg/kg). All dosing regimens were simulated on a Q3W treatment cycle.

TABLE 13
MORAb-202 parameter estimates of final PPK model

| Parameter | Estimate | % RSE | 95% CI |
|---|---|---|---|
| CL (L/hr) | 0.01595 | 3.03 | 0.01500-0.01689 |
| V1 (L) | 2.482 | 1.75 | 2.397-2.567 |
| Q (L/hr) | 0.008039 | 4.66 | 0.007304-0.008773 |
| V2 (L) | 1.875 | 8.35 | 1.568-2.183 |
| BW on CL (exponent) | 0.534 | 23.1 | 0.2919-0.7761 |
| BW on V1 (exponent) | 0.559 | 14.5 | 0.4006-0.7173 |
| Albumin on CL (exponent) | −0.7834 | 36.6 | −1.336-−0.2212 |
| Inter-Individual Variabilty | | | |
| CL (CV %) | 26.3 | 8.11 | 22.0-30.6 |
| V1 (CV %) | 15.5 | 6.83 | 13.2-17.4 |
| V2 (CV %) | 59.3 | 13.5 | 40.7-73.3 |
| R CL-V1 | 0.577 | 10.9 | 0.468-0.636 |
| Residual | | | |
| Proportional (CV %) | 13.7 | 3.58 | 12.8-14.7 |
| Additive (SD, ug/mL) | 0.0992 | 24.2 | 0.0521-0.1463 |

Eta shrinkage (%):
ETA_CL: 2.3%;
ETA_V1: 7.7%;
ETA_V2: 20.6%;
EPS shrinkage (%): 9.2%
% RSE = percent relative standard error of the estimate

### 1.3.1 Results

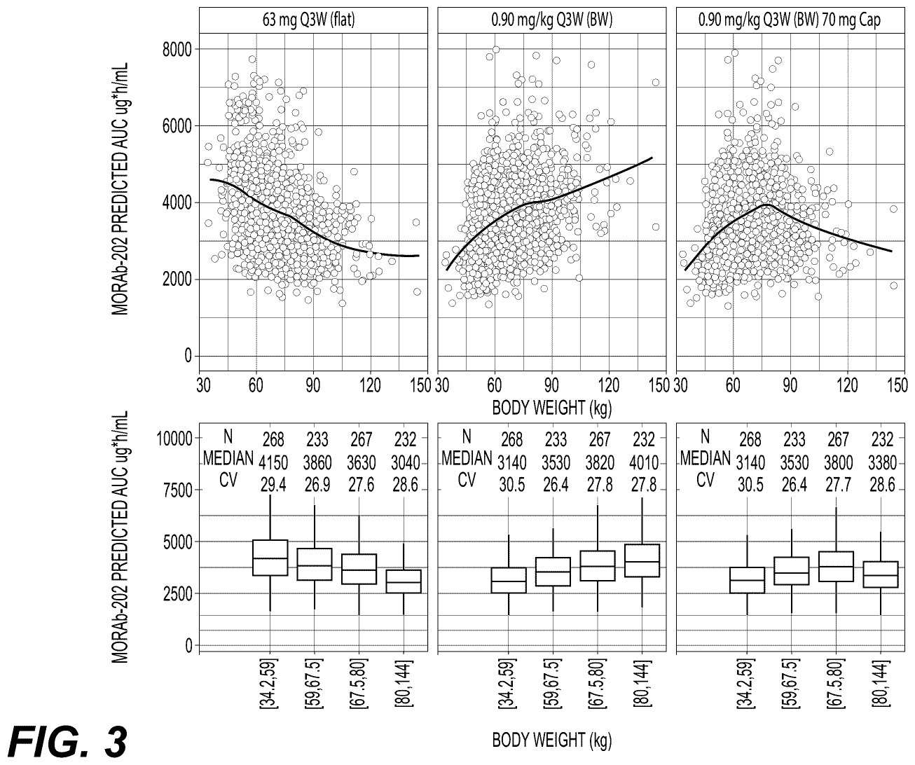

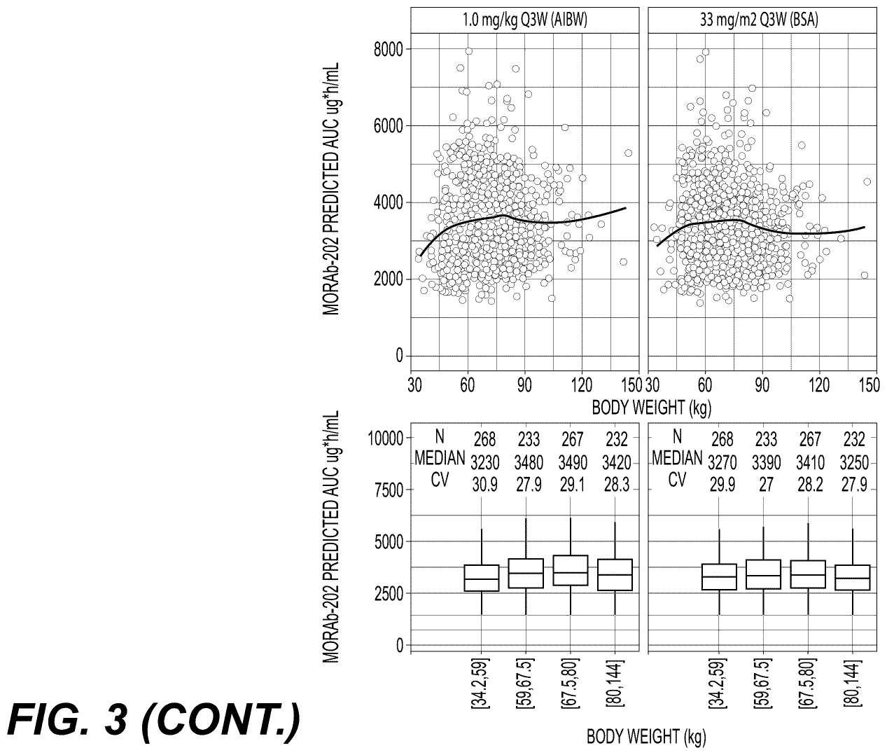

Results of the simulation are shown in FIG. 3. Dosing based on a flat dose was predicted to result in a 27% lower median AUC in subjects in the highest BW quartile compared to subjects in the lowest BW quartile. Dosing based on BW was predicted to result in a 28% higher median AUC in subjects in the highest BW quartile compared to subjects in the lowest BW quartile. Dosing regimens such as capping a BW-based dose at 70 mg, using an AIBW-based dose, or using a BSA-based dose were predicted to result in AUCs that were independent of body weight, thus reducing exposure levels of MORAb-202 in subjects with higher body weight. Predictions of AUC, Cₘₐₓ, ORR, and ILD for the clinically evaluated range of BW-based doses (described in sections 1.1 and 1.2) and equivalent BSA-based doses are provided in Table 6. A BSA-based dose of 33 mg/m² was predicted to provide a similar median exposure level as a BW-based dose of 0.9 mg/kg. A BSA-based dose of 25 mg/m² was predicted to provide a similar median exposure level as a BW-based dose of 0.68 mg/kg, while still offering a potential therapeutic benefit with a predicted ORR of approximately 24%.

TABLE 14
Predicted AUC, Cₘₐₓ, ORR, and ILD for MORAb-202 at body weight-
based doses and equivalent body surface area-based doses

| | | | AUC | Cₘₐₓ | | |
|---|---|---|---|---|---|---|
| | | | (μg*h/mL) | (μg/mL) | | |
| Dosing | | | Median | Median | ORR | ILD |
| regimen | Dose | | (90% PI) | (90% PI) | (90% PI) | (90% PI) |
| Body | 1.2 | mg/kg | 4750 | 32.1 | 0.666 | 0.768 |
| weight- | | | (2930, 7590) | (24, 43.7) | (0.313, 0.952) | (0.34, 0.973) |
| based | 0.9 | mg/kg | 3560 | 24.1 | 0.432 | 0.547 |
| dosing | | | (2200, 5690) | (18, 32.7) | (0.201, 0.811) | (0.21, 0.88) |
| | 0.68 | mg/kg | 2690 | 18.2 | 0.273 | 0.367 |
| | | | (1660, 4300) | (13.6, 24.7) | (0.14, 0.581) | (0.138, 0.722) |
| | 0.45 | mg/kg | 1780 | 12 | 0.152 | 0.213 |
| | | | (1100, 2850) | (8.98, 16.4) | (0.0935, 0.299) | (0.0812, 0.469) |
| Body | 44 | mg/m² | 4400 | 29.8 | 0.601 | 0.704 |
| surface | | | (2690, 6860) | (22.6, 39.5) | (0.273, 0.917) | (0.315, 0.956) |
| area- | 33 | mg/m² | 3310 | 22.4 | 0.383 | 0.49 |
| based | | | (2020, 5150) | (17, 29.5) | (0.179, 0.734) | (0.193, 0.844) |
| dosing | 25 | mg/m² | 2500 | 16.9 | 0.244 | 0.332 |
| | | | (1530, 3900) | (12.8, 22.3) | (0.128, 0.5) | (0.129, 0.665) |
| | 17 | mg/m² | 1660 | 11.2 | 0.14 | 0.194 |
| | | | (1010, 2570) | (8.42, 14.8) | (0.0874, 0.254) | (0.0779, 0.415) |

AUC = area under the curve; Cₘₐₓ = highest concentration of MORAb-202 in the blood after administration; ORR = objective response rate; ILD = interstitial lung disease; PI = prediction interval.
Values based on simulations (N = 1000 per dose in each dosing scenario).

### 1.4 Comparison of BSA- and BW-Based Dosing

Based on the clinical pharmacology assessments of evaluated BW-based doses (described in sections 1.1 and 1.2) and simulated results from different dosing regimens (described in section 1.3), a BSA-based dose of 33 mg/m² at Q3W was selected based on results from the simulation analysis showing that this dose would reduce ILD rate while maintaining a therapeutically meaningful benefit (i.e., ORR>30%). Additional simulations were performed to compare the predicted clinical outcomes of a BSA-based dose of 33 mg/m² with a BW-based dose of 0.9 mg/kg. All simulated comparisons were based on a Q3W treatment cycle.

### 1.41. Results

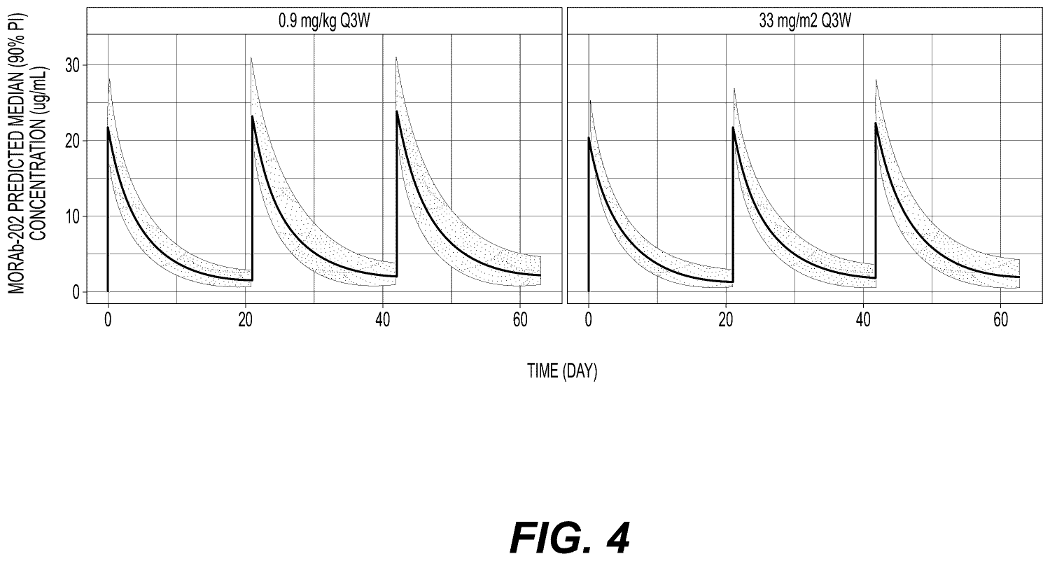

The predicted median concentrations of MORAb-202 over time on a BSA-based dose of 33 mg/m² were found to be similar to the median concentrations of MORAb-202 over the same time period on a BW-based dose of 0.9 mg/kg (FIG. 4). Predictions of AUC, Cₘₐₓ, ORR, and ILD across body weight quartiles were also similar for both dosing regimens (Table 15). Notably, a BSA-based of 33 mg/m² was predicted to scale exposure levels of MORAb-202 more evenly in subjects across BW quartiles, compared to a BW-based dose of 0.9 mg/kg. The BSA-based dosing approach maintained similar exposures in subjects in the lower BW quartiles as the BW-based dosing approach, while reducing exposures in subjects in the highest BW quartiles. As a result, a BSA-based dose of 33 mg/m² was predicted to result in a reduction in ILD rate by 18.4% for subjects in the highest BW quartile, compared to an equivalent BW-based dose of 0.9 mg/kg.

TABLE 15
Predicted MORAb-202 AUC, Cₘₐₓ, ORR, and ILD for 0.9 mg/kg Q3W and 33 mg/m² Q3W overall and across body weight quartiles

| | 0.9 mg/kg Q3W (BW-based dose) | | | | 33 mg/m² Q3W (BSA-based dose) | | | |
|---|---|---|---|---|---|---|---|---|
| | AUC | Cₘₐₓ | | | AUC | Cₘₐₓ | | |
| | (μg*h/mL) | (μg/mL) | | | (μg*h/mL) | (μg/mL) | | |
| | Median | Median | ORR | ILD | Median | Median | ORR | ILD |
| | (90% PI) | (90% PI) | (90% PI) | (90% PI) | (90% PI) | (90% PI) | (90% PI) | (90% PI) |
| Overall | 3560 | 24.1 | 0.432 | 0.547 | 3310 | 22.4 | 0.383 | 0.490 |
| (N = 1000) | (2200, 5690) | (18, 32.7) | (0.201, 0.811) | (0.21, 0.887) | (2020, 5150) | (17, 29.5) | (0.179, 0.734) | (0.193, 0.844) |
| BW Q1* | 3140 | 21.5 | 0.35 | 0.430 | 3270 | 22.5 | 0.376 | 0.458 |
| | (1800, 4990) | (16.4, 29.8) | (0.155, 0.708) | (0.171, 0.817) | (1910, 5310) | (17.6, 31.3) | (0.166, 0.759) | (0.181, 0.844) |
| BW Q2* | 3530 | 23.6 | 0.426 | 0.533 | 3390 | 22.8 | 0.398 | 0.505 |
| | (2220, 5260) | (18.2, 29.9) | (0.204, 0.751) | (0.224, 0.866) | (2150, 5060) | (17.6, 29.2) | (0.195, 0.719) | (0.202, 0.846) |
| BW Q3* | 3820 | 25.1 | 0.484 | 0.625 | 3410 | 22.5 | 0.402 | 0.542 |
| | (2330, 5730) | (18.6, 32.8) | (0.219, 0.815) | (0.264, 0.901) | (2030, 5180) | (17, 29.1) | (0.181, 0.74) | (0.222, 0.841) |
| BW Q4* | 4010 | 27.5 | 0.523 | 0.641 | 3250 | 21.6 | 0.371 | 0.457 |
| | (2480, 6170) | (20, 36.3) | (0.24, 0.864) | (0.262, 0.935) | (2000, 4750) | (16.1, 28.1) | (0.176, 0.667) | (0.197, 0.842) |

AUC = area under the curve; Cₘₐₓ = highest concentration of MORAb-202 in the blood after administration; ORR = objective response rate; ILD = interstitial lung disease; PI = prediction interval; BW = body weight; BSA = body surface area.
*BW Q1 (34.2, 59 kg); Q2 (>59, 67.5 kg); Q3 (>67.5, 80 kg); Q4 (>80, 144 kg)

### Example 2

### 2.1 Study Details

A multicenter, open-label phase 1/2 trial evaluating the safety, tolerability, and efficacy of MORAb-202, a folate receptor alpha (FRA)-targeting antibody-drug conjugate will be conducted with subjects with selected tumor types. The estimated duration of this Phase 1/2 study is approximately 2 years, with an enrollment period of approximately 15 months.

### 2.1.1 Objectives

There will be two parts to the trial: a Dose-Escalation part and a Dose-Confirmation part.

### 2.1.1.1 Primary Objectives

The primary objective of the Dose-Escalation part will be to evaluate safety and tolerability, and to determine the recommended Phase 2 dose (RP2D) of MORAb-202 in subjects with selected tumor types (ovarian cancer (OC), endometrial cancer (EC), non-small cell lung carcinoma (NSCLC), triple-negative breast cancer (TNBC)).

The primary objective of the Dose-Confirmation part will be to further evaluate the safety and tolerability of MORAb-202, and to evaluate preliminary efficacy measured by objective response rate (ORR) of MORAb-202 in subjects with OC and EC at selected doses.

### 2.1.1.2 Secondary Objectives

The secondary objectives of the trial are to: (i) evaluate duration of response (DOR), disease control rate (DCR), and clinical benefit rate (CBR), (ii) evaluate progression-free survival (PFS) and overall survival (OS), (iii) determine the pharmacokinetic (PK) profiles of MORAb-202, total antibody, and released eribulin in serum or plasma, and (iv) evaluate the relationship between folate receptor alpha (FRA) expression levels and clinical outcome measures to support the identification of an appropriate FRA cutoff point.

### 2.1.1.3 Exploratory Objectives

The exploratory objectives of the trial are to: (i) evaluate oxygen saturation using pulse oximetry for the detection and monitoring of interstitial lung disease (ILD), (ii) explore potential blood and tumor pharmacodynamics (PD) biomarkers (e.g., soluble FRA), and correlate with clinical outcome measures including PK, pharmacogenomics (PG), safety, and efficacy, (iii) investigate the effect of MORAb-202 on ventricular repolarization (Dose-Escalation Part only), (iv) evaluate the relationship between lines of prior therapy and clinical outcome measures (OC only), and (v) evaluate the utility of computer-based algorithms to objectively detect lung parenchyma patterns consistent with ILD (e.g., honeycombing, ground glass) in high resolution lung CT images for detection of potential predisposing pathology, changes indicating early ILD, as well as changes associated with ILD resolution.

### 2.2 Study Design

MORAb-202 will be administered as an intravenous (IV) infusion once every 3 weeks (21-day cycle). Treatment will stop upon intolerable toxicity, disease progression, or subject withdrawal for any reason.

### 2.2.1 Folate Receptor Alpha (FRA) Expression Analysis

All selected tumor types will be enrolled irrespective of the level of tumor FRA expression. However, FRA expression levels will be determined prospectively for analysis of correlation of FRA levels with efficacy outcome. Tumor samples to evaluate FRA expression levels (as described in the inclusion criteria of section 2.2.5) are required for study entry. Following completion of the Dose-Confirmation Part, an analysis will be conducted to identify a clinically meaningful FRA cut point.

### 2.2.2 Dose-Escalation

For the Dose-Escalation part, three doses are planned: 0.9, 1.2, and 1.6 mg/kg. Up to 6 subjects will be accrued at each dose level using a rolling 6 design. Subjects will have any of the 4 tumor types: OC, EC, NSCLC and TNBC.

Additional subjects with OC will be accrued to achieve approximately 10 such subjects at each dose level. These additional subjects will not be used for dose-escalation decisions, but their safety and efficacy data will contribute to the RP2D determination.

Other additional subjects may be accrued at selected dose levels, if necessary, for RP2D determination.

### 2.2.2.1 Rolling 6 Design

Dose level assignment is based on the number of subjects currently enrolled in the cohort, the number of dose-limiting toxicities (DLTs) observed, and the number of subjects at risk for developing a DLT (i.e., subjects enrolled but who are not yet assessable for toxicity). The rules for dose assignment are shown in the Dose Decision table for Rolling 6 design.

Subjects unevaluable for DLT will be replaced with the next available subject if escalation or de-escalation rules have not been fulfilled at the time the next available subject is enrolled onto the study.

### 2.2.2.2 Selection of RP2D

The RP2D will be determined based on an integrated assessment of safety, efficacy, PK, and PD data. The DLT and RP2D determinations will be agreed between the sponsor and investigators.

During the Dose-Confirmation Part of the study (described in section 2.2.3), toxicity will be monitored on an ongoing basis together with an independent data monitoring committee (IDMC). At any time, if there is concern with toxicity, the IDMC, investigators, and sponsor will re-evaluate the MORAb-202 dose and consider the appropriate action.

### 2.2.3 Dose-Confirmation

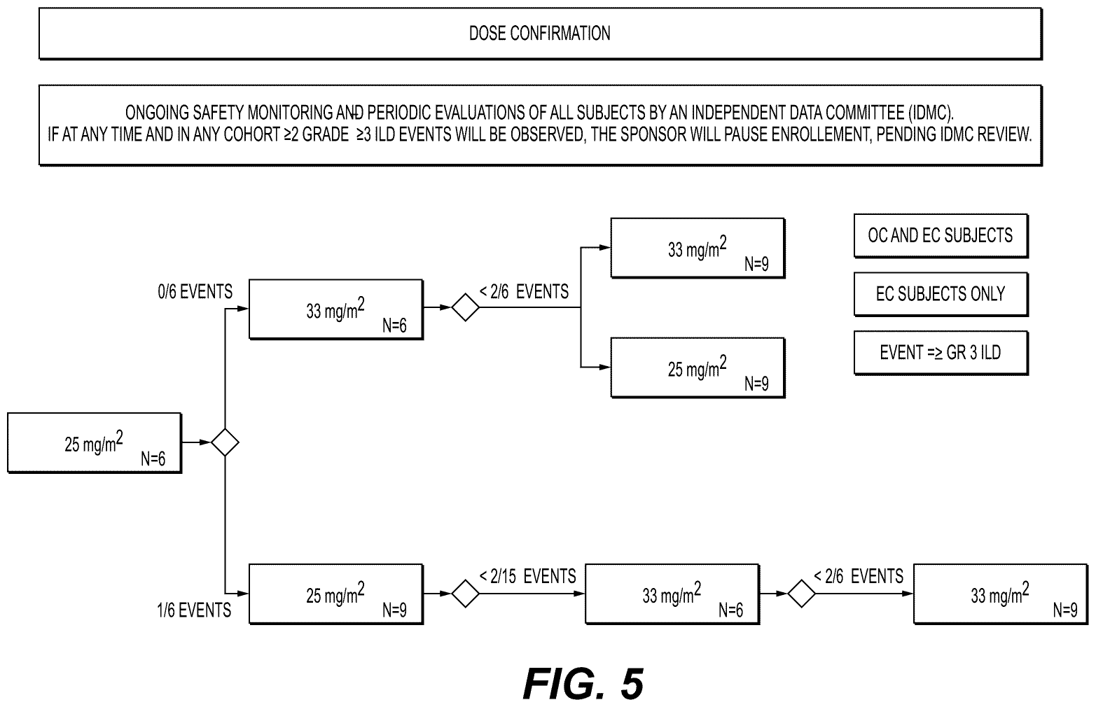

The Dose-Confirmation part will evaluate the safety and preliminary efficacy of MORAb-202 at selected dose levels in subjects with OC and EC. An overview of the study design is shown in FIG. 5.

There will be approximately 30 subjects in the Dose-Confirmation part in 4 study treatment cohorts. If at any time ≥2 Grade ≥3 ILD/pneumonitis (National Cancer Institute Common Terminology Criteria for Adverse Events [NCI CTCAE v5.0]) events are observed, the sponsor will pause enrollment, pending IDMC review. The initial cohort will enroll 6 subjects at 25 mg/m² of MORAb-202.

If there are no Grade 3 ILD events observed after all 6 subjects in the 25 mg/m² cohort have been evaluated (clinically and radiologically) for ILD for a minimum of 6 weeks after C1D1 (cycle 1 day 1), then 6 subjects will be enrolled in a second cohort at 33 mg/m². If there are <2 Grade ≥3 ILD events observed after all 6 subjects in the 33 mg/m² cohort have been evaluated (clinically and radiologically) for ILD for a minimum of 6 weeks after C1D1, then the study will proceed with 2 cohorts, 25 mg/m² and 33 mg/m², with 9 subjects randomly assigned to each cohort.

If there is 1 Grade ≥3 ILD event after dosing all 6 subjects in the 25 mg/m² cohort then this cohort will be expanded to enroll an additional 9 subjects (for a total of 15). After all 15 subjects have been observed for 6 weeks for evaluation of ILD, if there are <2 Grade ≥3 ILD events then enrollment will be opened at 33 mg/m² dose level for 6 subjects. If there are <2 Grade ≥3 ILD events in these 6 subjects, then this cohort will be expanded to enroll a total of 15 subjects.

The data will be analyzed to determine the regimen(s) acceptable for further investigation. The cohorts will be observed for ILD incidence and severity for the duration of the study. Efficacy will be assessed by ORR at Week 24.

The dosing regimen for this study is body surface area (BSA)-based dosing. The dose levels to be used in this portion of the study have been matched to body weight doses of 0.9 mg/kg and 0.68 mg/kg with BSA doses of 33 mg/m² and 25 mg/m² respectively. The BSA dose equivalents were confirmed using predictions from a MORAb-202 population PK model (described in Example 1). This revised dosing regimen has been implemented in the Dose-Confirmation part of this study due to the potential to reduce the ILD risk in higher body weight subjects whilst using doses within the therapeutic index of MORAb-202.

### 2.2.3.1 Study Period

There will be 3 periods for each subject: Pretreatment period, Treatment period, and Follow-up period.

Pretreatment Period: Days −28 to −1, CT/magnetic resonance imaging (MRI) scans must be performed within 28 days prior to study drug administration. All clinical and laboratory test results to determine eligibility must be performed within 7 days prior to study drug administration, unless otherwise indicated.

Treatment Period: MORAb-202 will be administered as an IV infusion once every 3 weeks (21-day cycle), otherwise known as Q3W. Subjects may stay on treatment until intolerable toxicity, disease progression, or subject withdrawal for any reason.

Follow-up Period: After discontinuation of study drug, see Duration of Treatment section for criteria for discontinuation, an off-treatment visit will be conducted after which all subjects will be followed for survival every 12 weeks for up to 3 years. Subjects coming off study treatment for reasons other than progressive disease (PD) should have tumor assessments performed according to the Schedule of Assessments from the date of the last assessment until disease progression is documented or the subject initiates another anticancer therapy, whichever occurs first, unless the study is terminated or the subject withdraws consent for follow-up. Data on subsequent anticancer therapy will be collected. If a subject discontinues study treatment due to ILD, or with ILD ongoing at the time of treatment discontinuation (eg, upon PD or adverse event [AE]), chest CT scans should continue (as per the protocol ILD assessment timepoints) until resolution or stabilization of the ILD (no worsening over 3 consecutive chest CT scans).

All subjects will be followed for survival for 3 years, except where a subject withdraws consent, or the sponsor chooses to halt survival follow-up after completion of the primary study analysis.

End of Study: The end of the study will be defined as last subject/last visit or the time that the sponsor terminates the study. An earlier data cutoff may occur for the purpose of preparing a primary clinical study report.

### 2.2.4 Number of Subjects

For the Dose-Escalation part of the study, approximately 36 subjects will be enrolled. For the Dose-Confirmation part of the study, approximately 30 subjects will be enrolled.

### 2.2.5 Inclusion Criteria

Subjects must meet all of the following criteria to be included in this study:
1. Aged ≥18 years;
2. For Dose-Escalation:
   Females (TNBC, EC and OC) or males/females (NSCLC adenocarcinoma). Subjects with the following disease characteristics:
      TNBC: Histologically confirmed diagnosis of metastatic TNBC (ie, estrogen receptor [ER] negative/progesterone receptor negative/human epidermal growth factor receptor 2 [HER2] negative (defined as IHC <2+ or fluorescence in situ hybridization [FISH] negative) breast cancer). Previously treated with at least one line of systemic anticancer therapy (cytotoxic or targeted anticancer agents) in the metastatic setting.
      NSCLC adenocarcinoma: Histologically or cytologically confirmed metastatic NSCLC adenocarcinoma: subjects who have failed previous treatment for metastatic disease, are not indicated or failed epidermal growth factor receptor (EGFR)-, ALK-, BRAF- or ROS1-targeted therapy, and for whom no alternative standard therapy exists.
      EC: Histologically confirmed diagnosis of advanced, recurrent or metastatic EC. Relapsed or failure of at least one platinum-based regimen or one immunotherapy-based regimen.
      Ovarian cancer or primary peritoneal cancer or fallopian tube cancer: Histologically confirmed diagnosis of high grade serous epithelial OC or primary peritoneal cancer or fallopian tube cancer. Subjects must have:
         platinum-resistant disease (defined as progression within 6 months after the last dose of at least 4 cycles of the last platinum containing chemotherapy regimen)
         received up to 4 lines of systemic therapy post development of platinum resistance.
For Dose-Confirmation:
   Ovarian cancer or primary peritoneal cancer or fallopian tube cancer:
      Platinum-resistant disease defined as:
         For participant with 1 line of platinum-containing therapy: RECIST v1.1 progression >1 month and <6 months after the last dose of the first platinum-containing chemotherapy regimen (of at least 4 cycles)
         For participant with 2-3 lines of platinum-containing therapy: RECIST v1.1 progression during or within 6 months after the last dose of the 2nd or 3rd platinum-containing chemotherapy regimen.
      Have received up to 3 prior lines of systemic therapy and for whom single-agent therapy is appropriate as the next line of therapy. Subjects may have been treated with up to one line of therapy subsequent to determination of platinum-resistance.
         Neoadjuvant±adjuvant will be considered 1 line of therapy.
         Maintenance therapy (eg, bevacizumab, PARP inhibitors) will be considered part of the preceding line of therapy (will not be counted as an independent line of therapy).
         Hormonal therapy will be counted as a separate line of therapy unless it was given as maintenance.
         Therapy changed due to toxicity in the absence of progression will be considered part of the same line.
      Subjects must have histologically confirmed diagnosis of advanced, recurrent, or metastatic EC. All histologies (including carcinosarcoma [no more than one subject per dose level]) and molecular subtypes will be included. Subjects may have been treated with an immune checkpoint inhibitor (ICI)-containing regimen (or be ineligible for ICI treatment) and must have had no more than 2 prior regimens (not including adjuvant therapy if progression or recurrent/metastatic disease occurred more than 6 months after the completion of the last cycle of adjuvant therapy).
      Note: There is no restriction regarding prior hormonal therapy.
3. Available tumor tissue for FRA expression (%) by IHC analysis as assessed by the vendor. There is no minimum requirement for FRA expression (%). However, the tumor sample must be evaluable for IHC analysis (ie, of sufficient quality and quantity). Sample re-submission will be permitted for subjects with tissue result of “non-evaluable” who are otherwise eligible. Tumor sample submission must be archival formalin-fixed, paraffin-embedded (FFPE) tissue block, or unstained slides sectioned within 45 days from the latest FFPE block, or a fresh biopsy sample obtained during screening but prior to initiation of study treatment.
4. Radiological disease progression on or after the most recent therapy by investigator assessment.
5. Measurable disease meeting the following criteria (confirmed by central radiographic review in the Dose-Confirmation Part only):
   At least one lesion of >1.0 cm in long axis diameter for non-lymph nodes or >1.5 cm in short axis diameter for lymph nodes that is serially measurable according to Response Evaluation Criteria in Solid Tumors (RECIST) v 1.1 using either CT or MRI.
   Lesions that have had external beam radiotherapy (EBRT) or loco-regional therapies such as radiofrequency (RF) ablation must show evidence of PD following radiotherapy based on RECIST 1.1 to be deemed a target lesion.
6. Eastern Cooperative Oncology Group Performance Status (ECOG PS) of 0 or 1.
7. Subjects who are expected to survive a minimum of 3 months after the first administration of the study drug.
8. Adequate renal function as evidenced by serum creatinine <1.5 mg/dL or calculated creatinine clearance ≥50 mL/minute according to a 12- or 24-hour urine collection.
9. Adequate bone marrow function, as evidenced by:
Absolute neutrophil count (ANC)≥1.0×10⁹/L
Hemoglobin (Hgb)≥9.0 g/dL
Platelet count ≥75×10⁹/L
Growth factors or transfusions, as per institutional practice, are allowed if needed to achieve the above values. Growth factor and platelet transfusion should not be used within 7 days of initiation of study treatment.
10. Adequate liver function, as evidenced by:
   Total bilirubin ≤1.5× upper limit of normal (ULN) except for unconjugated hyperbilirubinemia (eg, Gilbert's syndrome)
   Alanine aminotransferase (ALT) and aspartate aminotransferase (AST)≤3×ULN (in the case of liver metastases ≤5×ULN) unless there are bone metastases. Subjects with alkaline phosphatase (ALP)≤3×ULN, unless they and are known to have bone metastases in which case higher ALP values will also be allowed.
   Albumin >3.0 g/dL.
11. Subjects must undergo a washout period required from the end of prior treatment to the first administration of the study drug that will be as follows:
   Prior anticancer therapy:
      Prior chemotherapy, surgical therapy, radiation therapy: >3 weeks. Prior chest radiotherapy or pneumonectomy is an exclusion (see exclusion criteria in section 2.2.6).
      Antibody and other biologic therapeutic agents: ≥4 weeks.
      Endocrine therapy or, small-molecule targeted therapy: >2 weeks.
      Immunotherapy: ≥4 weeks.
12. Patients with a history of deep vein thrombosis (DVT) within 3 months prior must have completed at least 1 month of anticoagulation prior to starting study treatment. Anticoagulation must continue while on study treatment.
13. Patients at risk for DVT secondary to central venous catheters or with past medical history of DVT or clinical symptoms suggestive of DVT must have venous Doppler ultrasonography to rule out DVT during the screening period and prior to initiation of study treatment.
14. If a subject has undergone major surgery, the subject must have recovered adequately from the toxicity and/or complications from the intervention prior to starting study treatment.
15. Resolution of anticancer therapy-related or radiation-related toxicities to Grade 1 severity or lower, except for stable sensory neuropathy (Grade ≤2), anemia (Hgb≥9.0 g/dL), and alopecia (any grade).
16. Subject must be willing and able to comply with all aspects of the protocol.
17. Subject must provide written informed consent prior to any study-specific screening procedures.

### 2.2.6 Exclusion Criteria

Subjects who meet any of the following criteria will be excluded from this study:
1. Subjects with endometrial leiomyosarcoma, endometrial stromal sarcoma or high-grade sarcoma.
2. Subjects who received previous treatment with any folate receptor targeting agents.
3. Subjects with platinum refractory OC (defined as disease progression during or within four weeks of the last dose of the initial platinum-based chemotherapy treatment).
4. Currently enrolled in another clinical study or used any investigational drug or device, which in the opinion of the sponsor may interfere with the study treatment, within the past 28 days or 5 times the half-life (where prior drug therapy falls under the parameters of item 11 in the Inclusion Critera, these Inclusion Criteria should be followed) of any investigational drug preceding informed consent.
5. Subjects with brain or subdural metastases are not eligible, unless they have completed local therapy and have discontinued the use of corticosteroids for this indication for at least 2 weeks before starting treatment in this study. Any signs (e.g., radiologic) or symptoms of brain metastases must be stable for at least 4 weeks before starting study treatment.
6. Diagnosed with meningeal carcinomatosis.
7. Any other invasive malignancy that required treatment (other than definitive surgery) or has shown evidence of recurrence/progression (except for non-melanoma skin cancer, or histologically confirmed complete excision of carcinoma in situ) during the 2 years prior to starting study treatment.
8. Significant cardiovascular impairment. History within 6 months prior to the first dose of study drug of: congestive heart failure greater than New York Heart Association (NYHA) Class II; unstable angina; myocardial infarction; stroke; cardiac arrhythmia associated with hemodynamic instability.
9. Clinically significant ECG abnormality, including marked prolonged baseline QT as corrected using Fridericia's formula (QTcF) (repeated demonstration of a QTcF interval >500 ms). A history of risk factors for torsade de pointes (eg, heart failure, hypokalemia, family history of long QT Syndrome) or the use of concomitant medications that prolong the QTcF.
10. Known to be Human Immunodeficiency Virus (HIV) positive. Testing at entry not required.
11. Active viral hepatitis (B or C as demonstrated by positive serology). Testing at entry if there are no symptoms or history is not required unless as per local requirements.
12. Females who are breastfeeding or pregnant at Screening or Baseline (as documented by a positive beta human chorionic gonadotropin (ß-hCG) or human chorionic gonadotropin (hCG) with a minimum sensitivity of 25 IU/L or equivalent units of ß-hCG (or hCG). A separate baseline assessment is required if a negative screening pregnancy test was obtained more than 72 hours before the first administration of the study drug.
13. Females of childbearing potential who:
   within 28 days before study entry, did not use a highly effective method of contraception, which includes any of the following:
      total abstinence (if it is their preferred and usual lifestyle)* * Sexual abstinence is considered a highly effective method only if defined as refraining from heterosexual intercourse during the entire period of risk associated with the study intervention. The reliability of sexual abstinence needs to be evaluated in relation to the duration of the study and the preferred and usual lifestyle of the subject.
      an intrauterine device or intrauterine hormone-releasing system (IUS)
      a contraceptive implant
      an oral contraceptive (subject must be on a stable dose of the same oral contraceptive product for at least 28 days before dosing and throughout the study and for 90 days after study drug discontinuation)
      have a vasectomized partner with confirmed azoospermia
   do not agree to use a highly effective method of contraception (as described above) throughout the entire study period and for 90 days after study drug discontinuation.
For sites outside of the EU, it is permissible that if a highly effective method of contraception is not appropriate or acceptable to the subject, then the subject must agree to use a medically acceptable method of contraception, ie, double-barrier methods of contraception such as latex or synthetic condom plus diaphragm or cervical/vault cap with spermicide. NOTE: All females will be considered to be of childbearing potential unless they are postmenopausal (amenorrheic for at least 12 consecutive months, in the appropriate age group, and without other known or suspected cause) or have been sterilized surgically (ie, bilateral tubal ligation, total hysterectomy, or bilateral oophorectomy, all with surgery at least 1 month before dosing).
14. For Dose-Escalation only: Males who have not had a successful vasectomy (confirmed azoospermia) or they and their female partners do not meet the criteria above (ie, not of childbearing potential or practicing highly effective contraception throughout the study period and for 90 days after study drug discontinuation). If the female partner is pregnant, then males who do not agree to use latex or synthetic condoms throughout the study period and for 90 days after study drug discontinuation. No sperm donation is allowed during the study period and for 90 days after study drug discontinuation.
15. Pulmonary Function Test (PFT) abnormalities: FEV1/FVC<0.7, FEV1 or FVC<80%, DLCO<80%.
16. Current ILD/pneumonitis, or ILD/pneumonitis is suspected at screening or history of interstitial lung disease (ILD)/pneumonitis of any severity including ILD/pneumonitis from prior anticancer therapy.
17. Current infectious pneumonia, history of viral pneumonia.
18. Lung-specific clinically significant illnesses including, but not limited to any underlying pulmonary disorder (e.g., pulmonary embolism), asthma, chronic obstructive pulmonary disease (COPD), and restrictive lung disease.
19. Clinically significant pleural or pericardial effusion.
20. Prior pneumonectomy.
21. History of chest radiotherapy. Subjects with history of chest radiotherapy may be permitted if chest radiotherapy is documented >2 years before starting study treatment.
22. Any autoimmune, connective tissue, or inflammatory disorders with pulmonary involvement.
23. A known history of active TB (bacillus tuberculosis).
24. Scheduled for surgery during the study, other than minor surgery which would not delay study treatment.
25. An active clinically significant (in the opinion of the Investigator) infection requiring systemic therapy within 2 weeks prior to the first dose of study drug.
26. Administration of a live, attenuated vaccine within 4 weeks prior to the first dose of study drug, or anticipation that such a live attenuated vaccine will be required during the study. Inactivated vaccines (such as hepatitis A or polio vaccines) are permitted during the study. Seasonal influenza and COVID-19 vaccines that do not contain live virus are permitted.
27. Any prior hypersensitivity to monoclonal antibodies or contraindication to the receipt of corticosteroids or any of the excipients (investigators should refer to the prescribing information for the selected corticosteroid).
28. Known intolerance to either of the components of the study drug.
29. Any medical or other condition which, in the opinion of the investigator would preclude the subject's participation in the clinical study.
30. Receiving any medication prohibited in combination with the study treatment(s), as described in the product label for eribulin, unless medication was stopped within 7 days prior to enrollment.
31. Known psychiatric or substance abuse disorders that would interfere with cooperation with the requirements of the trial.

### 2.2.7 Study Treatment

Administration schedule: MORAb-202 will be administered once every 3 weeks as an IV infusion. One administration cycle is defined as 21 days (also known as Q3W). The concentration of the study drug in the vial is 10 mg/mL. The first infusion of MORAb-202 will be given over not less than 60 minutes. If no infusion reactions are observed, subsequent infusions can be infused as tolerated, but given over not less than 30 minutes.

### 2.2.8 Concomitant Drug/Therapy

Prophylaxis treatment of nosocomial infection, acceptable treatments for DLT events, or continuous treatment for complications of an AE is permitted with the provision that concomitant treatment should be kept to a minimum during this study. A G-CSF or equivalent may be used at the discretion of the investigator in accordance with institutional or national guidelines.

Radiation therapy to a symptomatic solitary non-target lesion may be allowed while on study treatment (up to twice) after consultation with the sponsor. Any brain lesion requiring radiation would be indicative of disease progression.

Acceptable treatments for adverse events, e.g., ILD, or continuous treatment for complications of an adverse event, e.g., ILD, are permitted. Acceptable treatments include administration of a corticosteroid, e.g., dexamethasone. Dexamethasone will be administered orally at 4 mg two times daily from Day 1 to Day 3 of each cycle of MORAb-202.

### 2.2.9 Assessments

Assessments will be conducted for efficacy, safety, PK, and PD.

### 2.2.10 Bioanalytical Methods

Serum MORAb-202 concentrations will be measured using a drug antibody ratio (DAR)-tolerant ligand-binding assay format designed to specifically quantify the toxin-conjugated antibody (DAR≥1) where any intact molecule with at least one DAR is detected. Serum total antibody concentrations will be measured using a validated ligand-binding assay format designed to detect farletuzumab, independent of the level of linker-toxin conjugation present (DAR≥0). Total eribulin concentrations will be measured using a validated liquid chromatography tandem mass spectrometry (LC-MS/MS) method. The antidrug antibody (ADA) will be measured using a validated ligand-binding assay.

### 2.2.11 Independent Data Monitoring Committee (IDMC)

Safety monitoring will be conducted by an IDMC. The function and membership of the IDMC will be described in the IDMC charter.

The first IDMC will occur after the first 6 subjects have been treated (first cohort) and observed for 6 weeks (timepoint of the first on study tumor assessment and first scheduled assessment for the evaluation of ILD), and then after each cohort. If a subject discontinues study treatment for any reason other than drug related toxicity prior to completing the first 6 weeks of study treatment, then that subject will be replaced. If ≥2 Grade ≥3 ILD/pneumonitis events are observed in the study, the sponsor will pause enrollment, pending IDMC review. The timing of IDMC reviews may also be adjusted or held more frequently if considered warranted by way of the sponsor's ongoing safety monitoring.

### 2.2.12 Sample Size Rationale

The primary objective of the Dose-Escalation Part is to evaluate the safety and tolerability, and to determine the RP2D of MORAb-202 in subjects with selected tumor types (OC, EC, NSCLC, TNBC). The sample size in this part will be approximately 36 subjects depending on the number of DLTs observed. Additional subjects with OC will be accrued, as a backfill, to achieve approximately 10 subjects with OC per dose level.

The primary objective of the Dose-Confirmation Part of this study is to evaluate the safety and preliminary efficacy of MORAb-202 in subjects with OC and EC. The planned number of subjects will be approximately 30. This part consists of cohorts of 6 or 9 subjects, with approximately 15 subjects at each dose level of 25 mg/m² and 33 mg/m² of MORAb-202. The order in which the cohort are carried out depends on the number of Grade ≥3 ILD events observed. The earlier cohorts will enroll both OC and EC subjects, and the last cohort(s) will enroll EC subjects only (please see Study Schema).

The data from these cohorts will be analyzed in order to determine the regimen(s) acceptable for further investigation. An acceptable regimen will be determined based on how well ILD is managed in these cohorts treated according to the methods described herein.

### Example 3

### 3.1 Study Design

A Phase 2 open-label, randomized, multicenter study is conducted assessing the safety, efficacy and tolerability of MORAb-202 in participants with metastatic NSCLC AC (adenocarcinoma). Participants will be randomized in a 1:1 ratio into 2 arms to receive MORAb-202 at 33 mg/m² in Arm A and 25 mg/m² in Arm B every 3 weeks.

The following participants will be enrolled in the study:

Participants without genomic alterations or with unknown genomic alterations in the metastatic setting after receiving:
Prior treatment with platinum-doublet chemotherapy and anti-PD-1/PD-L1, given either concurrently or sequentially
No more than 2 lines of systemic therapy (no more than 1 line of prior chemotherapy)

Participants with known genomic alteration in the metastatic setting after receiving:
At least one approved targeted therapy
No more than 3 lines of systemic therapy (no more than 1 line of chemotherapy)

Approximately 60 participants, will be randomized in a 1:1 ratio stratified by ECOG PS 0 versus 1 into 1 of the following treatment groups:
Arm A (N=30): MORAb-202 33 mg/m² Q3W
Arm B (N=30): MORAb-202 25 mg/m² Q3W

All participants will be treated until disease progression per RECIST v1.1 criteria as assessed by the investigator, unacceptable toxicity, participant withdrawal of consent for receiving study treatment, death, or the end of study, whichever occurs first. Maximum treatment duration will be up to 2 years; however, continuous safety and tumor assessment evaluation will guide the decision to treat a participant with additional cycles of study therapy beyond 2 years, if the participant has confirmed clinical benefit.

To further characterize safety and efficacy at the selected dose, up to an additional 30 participants may be enrolled in Arm A or Arm B in order to ensure at least 30 FRA-evaluable participants are enrolled in each cohort and approximately 25% of FRA-high expressors are enrolled. All relevant data (safety, efficacy, PK and PD) will inform the Sponsor's decision on subsequent clinical development, including enrollment of additional participants into an expansion cohort to assess the relationship with FRA expression through a protocol amendment or continuing development in a separate study.

The primary analysis will be performed when all participants in each arm have been treated and are followed up for a minimum of 6 months or discontinued earlier from treatment. One dose level of MORAb-202 will be selected at the primary analysis to continue further evaluation. The dose selection will be based on the totality of efficacy and safety data. Safety follow-up visits will be conducted 30 days after the last study drug administration. All ongoing treatment-related SAEs and ILD/pneumonitis events will be followed until resolution or stabilization. All participants discontinuing treatment for reasons other than disease progression will be followed for continued tumor imaging assessments until investigator-assessed disease progression per RECIST v1.1, death, or withdrawal of consent for tumor assessment, whichever occurs first. All participants will be followed for survival every 3 months until all randomized participants complete 2 years of survival follow-up.

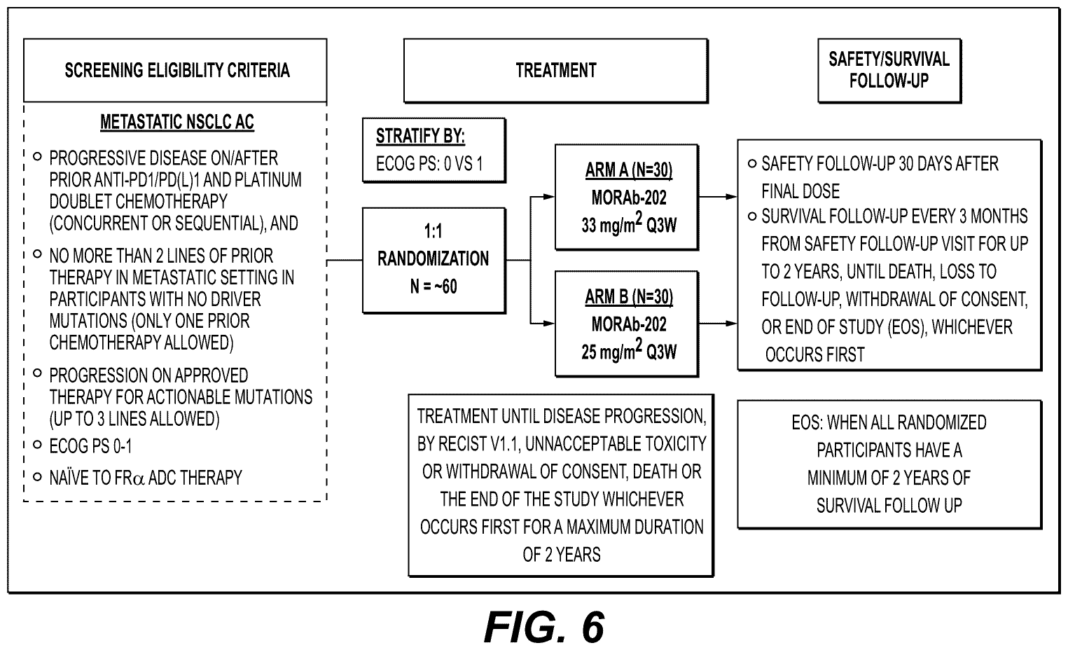

The study design schematic is presented in FIG. 6.

### 3.1.1 Objectives

### 3.1.1.1 Primary Objectives and Endpoints

Primary objectives of this study are: (i) to assess safety and tolerability of MORAb-202 in participants with previously treated non-small cell lung cancer (NSCLC) adenocarcinoma (AC), and (ii) to assess tumor response of MORAb-202 in participants with previously treated NSCLC AC.

Primary endpoints of this study are: (i) to evaluate incidence and severity of adverse events (AEs)/serious AEs (SAEs), treatment related AEs/SAEs, AEs leading to discontinuation, AEs of special interest (AESI), deaths and laboratory abnormalities, and (ii) to evaluate objective response rate (ORR) by Response Evaluation Criteria in Solid Tumors (RECIST) v1.1 per investigator assessment.

### 3.1.1.2 Secondary Objectives and Endopints

Secondary objectives of this study are: (i) to evaluate progression-free survival (PFS) of MORAb-202 in participants with previously treated NSCLC AC; (ii) to evaluate DCR of MORAb-202 in participants with previously treated NSCLC AC; and (iii) to evaluate duration of response (DoR) of MORAb-202 in participants with previously treated NSCLC AC who achieved a CR or PR.

Secondary endopoints of this study are: (i) to evaluate PFS by RECIST v1.1 per investigator assessment; (ii) to evaluate DCR by RECIST v1.1 per investigator assessment; and (iii) to evaluate DoR by RECIST v1.1 per investigator assessment.

### 3.2 Number of Subjects

Approximately 60 participants will be randomized in a 1:1 ratio to one of the 2 treatment groups with approximately 30 participants per arm. It is estimated that approximately 100 participants will be enrolled to achieve approximately 60 randomized participants, assuming a screen failure rate of approximately 40%. Randomized participants who are discontinued before receiving their first dose of MORAb-202 will be replaced.

### 3.3 Inclusion Criteria

Participants are eligible to be included in the study only if all of the following criteria apply:

### 1) Signed Written Informed Consent

a) Participants or legally acceptable representatives (LAR; where acceptable per local guidelines) must have signed and dated an Institutional Review Board (IRB)/Independent Ethics Committee (IEC)-approved written ICF in accordance with regulatory, local, and institutional guidelines. This must be obtained before the performance of any protocol-related procedures that are not part of normal patient care.
b) Participants must be willing and able to comply with scheduled visits, treatment schedule, laboratory testing, and other requirements of the study.

### 2) Type of Participant and Target Disease Characteristics

a) Histologically or cytologically documented metastatic NSCLC AC (as defined by the 8th International Association for the Study of Lung Cancer Classification).
b) Participants without genomic alterations or with unknown genomic alterations in the metastatic setting after receiving:
   i) Prior treatment with platinum-doublet chemotherapy and anti-PD-1/PD-L1, given either concurrently or sequentially
   ii) No more than 2 lines of systemic therapy (no more than 1 line of prior chemotherapy)
c) Participants with known genomic alteration in the metastatic setting:
   i) At least 1 approved targeted therapy
   ii) No more than 3 lines of systemic therapy (no more than 1 line of chemotherapy)
d) Investigator-assessed radiologically documented disease progression during or after last treatment
e) Measurable target disease assessed by the investigator according to RECIST v1.1.
f) Lesions that have had external beam radiotherapy (EBRT) or loco-regional therapies such as radiofrequency (RF) ablation must show evidence of disease progression based on RECIST v1.1 to be deemed a target lesion.
g) ECOG PS of 0 or 1
h) Resolution of toxicities from prior anti-cancer therapies to Grade 1 (NCI CTCAE v5.0) severity or lower before administration of study drug except for stable sensory neuropathy (Grade ≤2), anemia (hemoglobin [Hgb]≥9.0 g/dL), and long-lasting sequelae (such as alopecia, fatigue) that are not expected to interfere with study treatment.
i) Either FFPE tissue or newly obtained biopsies must be available for assessment by IHC at a central laboratory prior to randomization. The tumor sample (tissue block [preferred] or 15 unstained slides) must be evaluable for IHC analysis (ie, of sufficient quality and quantity) to meet eligibility criteria for randomization. Sample resubmission will be permitted for participants with tissue not of sufficient quality and quantity who are otherwise eligible.

### 3) Age of Participant

Participants (male or female) must be 18 years or older at the time of signing the informed consent.

### 4) Reproductive Status

Investigators shall counsel women of childbearing potential (WOCBP), and male participants who are sexually active with WOCBP, on the importance of pregnancy prevention, the implications of an unexpected pregnancy, and the potential of fetal toxicity occurring due to transmission of study intervention, present in seminal fluid, to a developing fetus, even if the participant has undergone a successful vasectomy or if the partner is pregnant.
The investigator shall evaluate the effectiveness of the contraceptive method in relationship to the first dose of study intervention.
Local laws and regulations may require the use of alternative and/or additional contraception methods.
a) Female Participants:
   i) Female participants must have documented proof in their medical documents that they are not of childbearing potential.
   ii) Women who are not of childbearing potential are exempt from contraceptive requirements.
   iii) WOCBP must have a negative highly sensitive serum or urine pregnancy test (minimum sensitivity 25 IU/L or equivalent units of HCG) within 24 hours prior to the start of study intervention.
      (1) If a urine test cannot be confirmed as negative (eg, an ambiguous result), a serum pregnancy test is required. In such cases, the participant must be excluded from participation if the serum pregnancy result is positive.
      (2) The investigator is responsible for review of medical history, menstrual history, and recent sexual activity to decrease the risk for inclusion of a woman with an early undetected pregnancy.
   iv) WOCBP must agree to follow instructions for method(s) of contraception described below and included in the ICF.
      WOCBP are permitted to use hormonal contraception methods.
   v) A female participant is eligible to participate if she is not pregnant or breastfeeding, and at least 1 of the following conditions applies:
      (1) Is not a WOCBP
         OR
      (2) Is a WOCBP and using a contraceptive method that is highly effective (with a failure rate of <1% per year), with low user dependency, during the intervention period and for at least 28 days before dosing and throughout the study and for 90 days after MORAb-202 discontinuation and agrees not to donate eggs (ova, oocytes) for the purpose of reproduction for the same time period.
b) Male Participants:
Males who are sexually active with WOCBP must agree to follow instructions for method(s) of contraception as described below.
   i) Azoospermic males are not exempt from contraceptive requirements and will be required to always use a latex or other synthetic condom during any sexual activity (eg, vaginal, anal, oral) with WOCBP, even if the participant has undergone a successful vasectomy or if the partner is pregnant.
   ii) Male participants will be required to always use a latex or other synthetic condom during any sexual activity (eg, vaginal, anal, oral) with WOCBP, even if the participants have undergone a successful vasectomy or if their partner is already pregnant or breastfeeding. Males should continue to use a condom during the intervention period and for at least 28 days before dosing and throughout the study and 90 days after MORAb-202 discontinuation.
   iii) Female partners of males participating in the study should be advised to use highly effective methods of contraception during the intervention period and for at least 90 days after the last dose of study intervention in the male participant.
   iv) Male participants with a pregnant or breastfeeding partner must agree to remain abstinent from sexual activity or use a male condom during any sexual activity (eg, vaginal, anal, oral), even if the participants have undergone a successful vasectomy, during the intervention period and for at least 90 days after the last dose of study intervention.
   v) Male participants must refrain from donating sperm during the intervention period and for at least 90 days after the last dose of study intervention.
   vi) Breastfeeding partners should be advised to consult their health care providers about using appropriate highly effective contraception during the time the participant is required to use condoms.

### 3.4 Exclusion Criteria

Participants are excluded from the study if any of the following criteria apply:

### 1) Medical Conditions

a) NSCLC histologies other than AC (ie, squamous cell carcinoma; large cell carcinoma).
b) Pulmonary Function Test (PFT) abnormalities: Forced expiratory 1 (FEV1)<70%, or forced vital capacity (FVC)<60%, diffusing capacity of the lung for carbon monoxide (DLCO)<80%.
c) Lung-specific clinically significant illnesses that are not managed by medication including, but not limited to, any underlying pulmonary disorder (eg, pulmonary embolism), asthma, chronic obstructive pulmonary disease (COPD), and restrictive lung disease.
d) Clinically significant pleural or pericardial effusion requiring drainage or ascites requiring peritoneal shunt or CART (Concentrated Ascites Reinfusion Therapy)
e) Prior pneumonectomy. Prior lobectomy and segmentectomy are allowed >12 months before treatment.
f) Recent chest radiotherapy. Participants with chest or chest wall radiation may be permitted if chest radiation is documented >12 months before starting study treatment.
g) Current infectious pneumonia, history of viral pneumonia (including COVID-19-related infection) with evidence of persistent radiologic abnormalities.
i) Previous SARS-CoV-2 infection, either suspected or confirmed within 4 weeks prior to randomization. Additionally, acute symptoms must have completely resolved and based on investigator assessment in consultation with the BMS Medical Monitor (or designee), there are no sequelae that would place the participant at a higher risk of receiving investigational treatment.
   ii) Participants currently in interventional trials for coronavirus disease 2019 (COVID-19), may not participate in BMS clinical studies until the specific washout period is achieved. If a study participant has received an investigational COVID-19 vaccine or other IP designed to treat or prevent COVID-19 prior to screening, enrollment must be delayed until the biologic impact of the vaccine or IP is stabilized, as determined by documented discussion between the investigator and the Medical Monitor (or designee).
   NOTE: COVID-19 polymerase chain reaction (PCR) viral testing may be required prior to randomization based on specific country/regional guidelines, and the result of this testing may impact study participation. Testing results should be discussed with the BMS Medical Monitor (or designee) to confirm eligibility.
h) Investigator assessed current ILD/pneumonitis, or ILD/pneumonitis is suspected at screening or history of ILD/pneumonitis of any severity including ILD/pneumonitis from prior anticancer therapy.
i) Spinal cord compression or untreated, symptomatic central nervous system (CNS) metastases (brain or leptomeningeal). Participants are eligible if CNS metastases are asymptomatic and do not require immediate treatment or if these have been treated and there is no MRI or CT evidence of progression for at least 4 weeks after treatment is complete and within 28 days prior to first dose of study treatment and participants have neurologically returned to baseline (except for residual signs or symptoms related to the CNS treatment). In addition, participants must have discontinued anticonvulsant therapy and must have discontinued corticosteroids, or be on a stable or decreasing dose of ≤10 mg daily prednisone (or equivalent) for at least 2 weeks prior to treatment. Imaging performed within 28 days prior to treatment must document radiographic stability of CNS lesions and be performed after completion of any CNS-directed therapy.
j) Participants with a condition requiring systemic treatment with either corticosteroids >10 mg daily prednisone equivalents or other immunosuppressive medications within 14 days of study treatment administration, except for steroid adrenal replacement where doses of >10 mg daily prednisone equivalent, are allowed in the absence of active autoimmune disease.
   i) Treatment with a short (<5 days) course of steroids up to 7 days prior to initiating study treatment is permitted.
k) Evidence of active infection including tuberculosis and uncontrolled infection requiring systemic antibacterial, antiviral, or antifungal therapy ≤14 days prior to treatment.
   i) Uncontrolled or significant cardiovascular conditions within 6 months prior to enrollment, including, but not limited, to any of the following:Cardiac angioplasty or stenting, myocardial infarction, unstable angina, coronary artery bypass graft surgery, symptomatic peripheral vascular disease, class III or IV congestive heart failure (as defined by the New York Heart Association), pericarditis, or myocarditis.
   ii) Ongoing symptomatic cardiac dysrhythmias, history of clinically significant arrhythmias (such as ventricular tachycardia, ventricular fibrillation, or torsades de pointes).
l) Clinically significant ECG abnormality, including marked prolonged baseline QTcF (repeated demonstration of a QTcF interval >500 msec). A history of risk factors for torsade de pointes (eg, heart failure, hypokalemia, family history of long QT Syndrome) or the use of concomitant medications that prolong the QTcF.
m) Evidence of active bleeding or medically significant hemorrhage within 3 months prior to enrollment.
n) History of deep vein thrombosis (DVT) within 6 weeks prior to enrollment. Participants who completed at least 1 month of anticoagulation prior to starting study treatment and continue while on study are eligible.
   i) Participants at risk for DVT secondary to central venous catheters or with past medical history of DVT or clinical symptoms suggestive of DVT must have venous Doppler ultrasonography to rule out DVT during the screening period and prior to initiation of study treatment.
o) Any autoimmune, connective tissue, or inflammatory disorders (eg, rheumatoid arthritis, Sjögren's syndrome, sarcoidosis, etc) where there is documented (or suspicion of) pulmonary involvement.
p) Concurrent malignancy (present during screening) requiring treatment, or history of prior malignancy active within 2 years prior to randomization, except for the NSCLC under study (ie, participants with a history of prior malignancy are eligible if treatment was completed at least 2 years before randomization and the participant has no evidence of disease). Participants with history of prior early stage basal/squamous cell skin cancer or non-invasive or in situ cancers (ie, superficial bladder cancer, carcinoma in situ of the prostate, cervix, or breast) that have undergone definitive treatment at any time are also eligible.
q) Any condition including medical, emotional, psychiatric, or logistical that, in the opinion of the investigator, would preclude the participant from adhering to the protocol or would increase the risk associated with study participation or study drug administration or interfere with the interpretation of safety results.

### 2) Prior/Concomitant Therapy

a) Participants who have received prior investigational treatment with FRA-targeting agent, or FRA-targeting ADCs including MORAb-202.
b) Any condition requiring folate supplementation (eg, folate deficiency).
c) Participants with known intolerance to components of the study drug.
d) Currently enrolled in another clinical study or used any investigational drug or device, which in the opinion of the sponsor may interfere with the study treatment, within the past 28 days or 5 times the half-life of the investigational drug (whichever is longer) prior to the start of the study treatment.
e) Any major surgery within 4 weeks of the first dose of study treatment. Participants must have recovered from the effects of major surgery or significant traumatic injury at least 14 days before the first dose of study treatment.
f) Treatment with any live/attenuated vaccine within 30 days of first study treatment.

### 3) Physical and Laboratory Test Findings

a) Evidence of organ dysfunction or any clinically significant deviation from normal in physical examination, vital signs, ECG, or clinical laboratory determinations beyond what is consistent with the target population
   i) Inadequate renal function as evidenced by serum creatinine >1.5 mg/dL or calculated creatinine clearance (CrCL)<50 mL/minute according to a 12- or 24-hour urine collection.
   ii) Inadequate bone marrow function, as evidenced by:
      (1) Absolute neutrophil count (ANC)<1.0×108T⁹8T/L
      (2) Hgb<9.0 g/dL
      (3) Platelet count <75×10⁹/L
   NOTE: Supportive therapies as blood/platelet transfusion, hematopoietic stimulating agent including granulocyte colony stimulating factor (G-CSF) formulation needed to achieve the above values are allowed, as per institutional practice, if given ≥1 week before study treatment.
   iii) Inadequate liver function, as evidenced by:
      (1) Total bilirubin >1.5× upper limit of normal (ULN) except for unconjugated hyperbilirubinemia (eg, Gilbert's syndrome, who must have a total bilirubin <3×ULN).
      (2) ALT and aspartate aminotransferase (AST)>3×ULN (in the case of liver metastases >5×ULN) unless there are bone metastases. Participants with alkaline phosphatase (ALP)>3×ULN, unless they and are known to have bone metastases in which case higher ALP values will also be allowed.
   iv) Serum albumin <3.0 g/dL.
b) Known human immunodeficiency virus (HIV) positive with an AIDS defining opportunistic infection within the last year, or a current CD4 count <350 cells/μL. Participants with HIV are eligible if:
   i) They have received antiretroviral therapy (ART) for at least 4 weeks prior to randomization as clinically indicated while enrolled on study
   ii) They continue on ART as clinically indicated while enrolled on study
   iii) CD4 counts and viral load are monitored per standard of care by a local health care provider
NOTE: Testing for HIV must be performed at sites where mandated locally. HIV positive participants must be excluded where mandated locally.
c) Active viral hepatitis B or C as evidenced by the following:
   i) Any positive test result for hepatitis B virus (HBV) indicating presence of virus, eg, hepatitis B surface antigen (HBsAg, Australia antigen) positive.
   ii) Any positive test result for hepatitis C virus (HCV) indicating presence of active viral replication (detectable HCV-ribonucleic acid [RNA]). Note: Participants with positive HCV antibody and an undetectable HCV RNA are eligible to enroll.
   iii) Additional testing or substitute testing per institutional guidelines to rule out infection is permitted.

### 4) Allergies and Adverse Drug Reaction

a) Has any prior severe hypersensitivity (Grade ≥3) to monoclonal antibodies or eribulin, or contraindication to the receipt of corticosteroids or any of the excipients.

### 5) Other Exclusion Criteria

a) Prisoners or participants who are involuntarily incarcerated. (Note: Under certain specific circumstances and only in countries where local regulations permit, a person who has been imprisoned may be included or permitted to continue as a participant. Strict conditions apply, and BMS approval is required.)
b) Participants who are compulsorily detained for treatment of either a psychiatric or physical illness (eg, infectious disease).

### 3.5 Study Interventions

MORAb-202 will be administered to subjects according to Table 16 below:

TABLE 16
Study interventions in CA116003

| Name | Arm A | Arm B |
|---|---|---|
| Intervention | MORAb-202/10 | MORAb-202/10 |
| Name/ Unit | mg/ml | mg/ml |
| Dose Strength(s) | | |
| Type | Drug | Drug |
| Dose Formulation | Lyophilized powder in | Lyophilized powder in |
| | a Single Use Vial | a Single Use Vial |
| Dosage Level(s) | 33 mg/m² Q3W | 25 mg/m² Q3W |
| Route of | IV infusion | IV infusion |
| Administration | | |
| Use | Experimental | Experimental |
| IMP and NIMP/AxMP | IMP | IMP |
| Sourcing | Provided centrally by | Provided centrally by |
| | the Sponsor | the Sponsor |

AxMP, auxiliary medicinal product; IMP, investigational medicinal product; IV, intravenous; NIMP, non-investigational medicinal product; Q3W, every 3 weeks.

### 3.6 Dosage Modifications

For participants who experience toxicity but meet criteria for dose modification, the next administration of MORAb-202 should be reduced 1 dose level lower. Details are shown in Table 17. Consultation with the study Medical Monitor is required prior to dose reduction. Once a dose is decreased, it cannot be increased again.

TABLE 17
Dosage modifications for subjects who experience toxicity

MORAb-202

| Dose levels | Arm A | Arm B |
|---|---|---|
| Starting dose | 33 mg/m² | 25 mg/m² |
| First dose reduction (Dose level −1) | 25 mg/m² | 17 mg/m² |

### Example 4

### 4.1 Study Details

A phase 2, open-label, randomized, multicenter study will be conducted that will evaluate the safety, efficacy, and tolerability of MORAb-202 in female participants with platinum-resistant high grade serous (HGS) ovarian, primary peritoneal, or fallopian tube cancer. The study research hypothesis is that MORAb-202 will have a favorable benefit-risk profile at either 33 mg/m² or 25 mg/m², compared to IC chemotherapy, as measured by overall response rate (ORR) and safety profile in participants with platinum resistant high-grade serous ovarian cancer, primary peritoneal cancer, or fallopian tube cancer. The study duration will be approximately 4 years.

### 4.1.1 Objectives

### 4.1.1.1 Primary Objectives

The primary objectives of the study are to: (i) compare objective response rate of MORAb-202 vs Investigator's Choice (IC) chemotherapy (in all randomized participants); and (ii) evaluate the proportion of participants with treatment-related adverse events (TRAEs) leading to discontinuation in each arm within 6 months from first dose of study drug administration in all treated participants.

The endpoints of the primary objectives described above are: (i) objective response rate (ORR) by RECIST v1.1 per Investigator assessment; and (ii) TRAEs leading to discontinuation.

### 4.1.1.2 Secondary Objectives

The secondary objectives of the study are to: (i) evaluate disease control rate (DCR) of MORAb-202 and IC chemotherapy in all randomized participants; (ii) evaluate duration of response (DoR) of MORAb-202 and IC chemotherapy in all randomized participants; and (iii) evaluate progression-free survival (PFS) of MORAb-202 and IC chemotherapy in all randomized participants. The endpoints of the secondary objectives described above are: (i) DCR by RECIST v1.1 per Investigator assessment; (ii) DoR by RECIST v1.1 per Investigator assessment; and (iii) PFS by RECIST v1.1 per Investigator assessment.

### 4.2 Study Design

Participants will be randomized in a 2:2:1 ratio into the following treatment arms:
Arm A (N=60): Administration of MORAb-202 at 33 mg/m² (with the option for dose reduction to 25 mg/m²) once every 3 weeks (21-day cycle).
Arm B (N=60): Administration of MORAb-202 at 25 mg/m² (with the option for dose reduction to 17 mg/m²) once every 3 weeks (21-day cycle).
Arm C (N=30): Administration of IC single-agent chemotherapy selected from the following:
   Paclitaxel at 80 mg/m² IV on Days 1, 8, 15, and 22 of a 28-day cycle; or
   Pegylated liposomal doxorubicin (PLD) at 40 mg/m² IV on Day 1 of a 28-day cycle; or
   Topotecan at 4 mg/m² IV on Days 1, 8, and 15 of a 28-day cycle or 1.25 mg/m² on 5 consecutive days on Days 1 to 5 of a 21-day cycle.

MORAb-202 will be administered as an IV infusion.

Participants will be randomized by FRA expression (≥75% tumor staining vs<75% tumor staining) and number of prior lines of therapy (1 vs 2-3).

All participants will be treated until disease progression as assessed by the Investigator according to RECIST v1.1, unacceptable toxicity, participant withdrawal of consent for receiving study treatment, death, or the end of study, whichever occurs first. Maximum treatment duration will be up to 2 years; however, continuous safety and tumor assessment evaluation will guide the decision to treat a participant with additional cycles of study therapy beyond 2 years, if the participant has a confirmed clinical benefit.

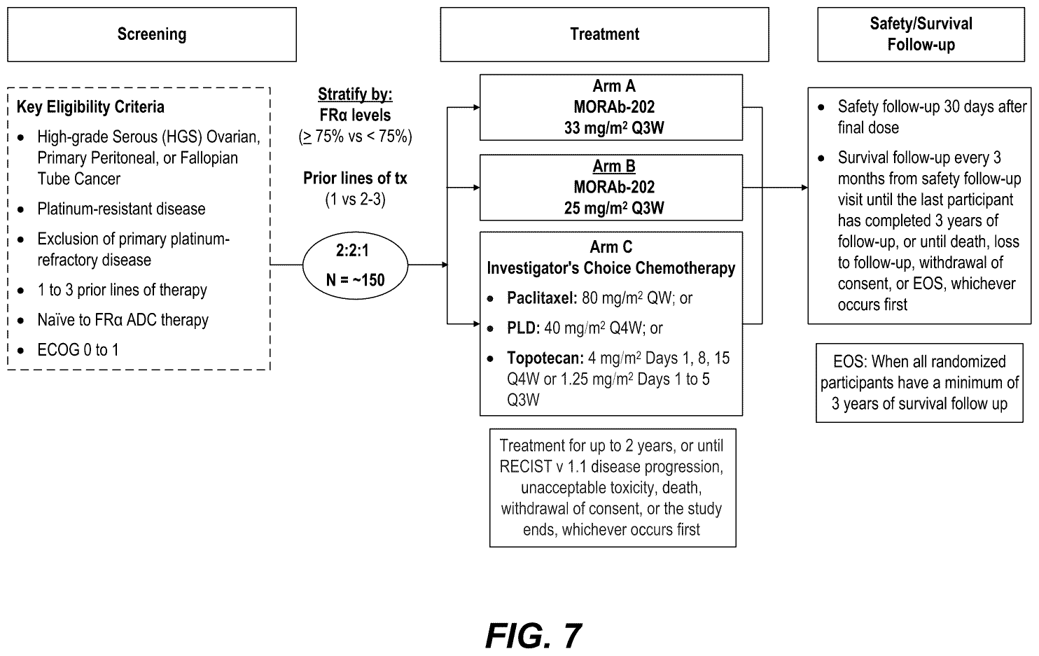

An overview of the study design is shown in FIG. 7.

### 4.3 Number of Participants

Approximately 150 participants will be randomized in a 2:2:1 ratio to 1 of the 3 treatment arms, with approximately 60 participants per MORAb-202 arm (Arms A and B) and 30 participants in the chemotherapy arm (Arm C). It is estimated that approximately 200 participants will be enrolled to achieve approximately 150 randomized participants, assuming a screen failure rate of approximately 25%.

### 4.4 Inclusion Criteria

The main inclusion criteria for participants in the study are as follows. All of the following must apply for a participant to be eligible to be included in the study:
Female participants with histologically confirmed diagnosis of HGS ovarian, primary peritoneal, or fallopian tube cancer.
Platinum-resistant disease, defined as:
   For participants who had only 1 line of platinum-based therapy: progression between >1 month and ≤6 months after the last dose of platinum-based therapy of at least 4 cycles.
   For participants who had 2 or 3 lines of platinum-based therapy: progression ≤6 months after the last dose of platinum-based therapy.
Participants have received at least 1 but no more than 3 prior lines of systemic therapy and for whom single-agent therapy is appropriate as the next line of therapy. Participants may have been treated with up to 1 line of therapy subsequent to determination of platinum-resistance.
   Participants must have received prior treatment with bevacizumab or must be deemed medically inappropriate or ineligible/intolerant to receive bevacizumab, refused to receive bevacizumab, or been unable to receive bevacizumab due to lack of access.
   Note: (i) Neoadjuvant+adjuvant chemotherapy will be considered 1 line of therapy. (ii) Maintenance therapy (eg, bevacizumab, PARP inhibitors) will be considered part of the preceding line of therapy. (iii) Therapy changed in the absence of progression will be considered part of the same line.
Disease progression per RECIST v1.1 (by Investigator assessment) of at least one measurable lesion on or after the most recent therapy. Either formalin-fixed, paraffin-embedded (FFPE) tissue (up to 5 years old) or newly obtained biopsies must be available for FRA assessment prior to randomization. The tumor sample (preferably a tissue block or a minimum of 15 unstained slides) must be evaluable for FRA IHC analysis to meet eligibility criteria. Sample resubmission will be permitted for participants with non-evaluable FRA IHC who are otherwise eligible.
Eastern Cooperative Oncology Group Performance Status (ECOG PS) of 0 or 1.
Participant must be ≥18 years of age (or the legal age of consent in the jurisdiction in which the study is taking place) at the time of signing the informed consent form (ICF).

### 4.5 Exclusion Criteria

Participants are excluded from the study if any of the following apply:
Clear cell, mucinous, endometrioid or sarcomatous histology, or mixed tumors containing components of any of these histologies, or low grade or borderline ovarian cancer.
Primary platinum-refractory ovarian cancer defined as disease progression within 1 month of the last dose of the first line platinum-containing regimen.
Pulmonary function test (PFT) abnormalities: FEV1<70% or FVC<60%, and DLCO <80%.
Investigator-assessed current ILD/pneumonitis, or ILD/pneumonitis suspected at Screening or history of ILD/pneumonitis of any severity including ILD/pneumonitis from prior anti-cancer therapy.
Current infectious pneumonia, history of viral pneumonia (including COVID-19-related infection) with evidence of persistent radiologic abnormalities.
Significant third-space fluid retention (eg, ascites or pleural effusion) that requires repeated drainage.
Clinically significant pericardial effusion requiring drainage.
Prior pneumonectomy. Prior lobectomy and segmentectomy are allowed >12 months before treatment.
Recent chest radiotherapy. Participants with chest or chest wall radiation (eg, history of breast cancer) may be permitted if the radiation is documented >6 months before starting study treatment.
Any autoimmune, connective tissue, or inflammatory disorders (eg, rheumatoid arthritis, Sjögren's syndrome, sarcoidosis, etc) where there is documented (or suspicion of) pulmonary involvement.
Spinal cord compression or untreated, symptomatic central nervous system (CNS) metastases. Participants are eligible if CNS metastases are asymptomatic and do not require immediate treatment, or have been treated and participants have neurologically returned to baseline (except for residual signs or symptoms related to the CNS treatment). In addition, participants have discontinued anticonvulsant therapy and must have been either off corticosteroids, or on a stable or decreasing dose of ≤10 mg daily prednisone (or equivalent) for at least 2 weeks prior to treatment. Imaging performed within 28 days prior to treatment must document radiographic stability of CNS lesions and be performed after completion of any CNS-directed therapy.
Concurrent malignancy (present during screening) requiring treatment, or history of prior malignancy active within 2 years prior to randomization (ie, participants with a history of prior malignancy are eligible if treatment was completed at least 2 years before randomization and the patient has no evidence of disease). Participants with history of prior early stage basal/squamous cell skin cancer or non-invasive or in situ cancers that have undergone definitive treatment at any time are also eligible.
Participants with a condition requiring systemic treatment with either corticosteroids >10 mg daily prednisone equivalents or other immunosuppressive medications within 14 days of study treatment administration, except for steroid adrenal replacement where doses of >10 mg daily prednisone equivalent, are allowed in the absence of active autoimmune disease.
   Treatment with a short course (<5 days) of steroids up to 7 days prior to initiating study treatment is permitted.

### 4.6 Study Interventions

Compounds will be administered to participants according to Table 18 below:

TABLE 18
Study interventions for CA116001

| Intervention Name | Unit Dose Strength(s) | | IMP/Non-IMP/AxMP |
|---|---|---|---|
| MORAb-202 | 10 | mg/mL | IMP |
| Lyophilized | | | |
| Powder in a Single | | | |
| Use Vial | | | |
| Paclitaxel | 6 | mg/mL | IMP |
| Pegylated Liposomal | 2 | mg/mL | IMP |
| Doxorubicin (PLD) | | | |
| Topotecan | 1 | mg/mL | IMP |

Abbreviations: AxMP, auxiliary medicinal product; IMP, investigational medicinal product; PLD, pegylated liposomal doxorubicin.

### Example 5

5.1 Preliminary Results from Phase 1/2 Trial (Example 2)

The dose evaluation part of the study was designed with two sequential cohorts at 25 mg/m² and 33 mg/m². Enrollment has been completed in the two sequential cohorts. A total of 14 subjects were enrolled and treated with MORAb-202 with 7 subjects in each cohort. Further investigation will continue at 25 mg/m² Q3W. Acceptable safety and initial anti-tumor activity has been demonstrated with this regimen. Mean PK profiles of 25 mg/m² for MORAb-202, total antibody, and released eribulin were comparable to those of a 0.68 mg/kg dose. BSA-based dosing decreased the total amount of drug (mg) compared with BW-based estimated total dose in most subjects. Additional testing is planned for every two weeks and for once per week dosing regimens.

Selected sequences:

```
SEQ ID NO: 1 (MORAb-003 HC CDR1; Kabat): GYGLS
SEQ ID NO: 2 (MORAb-003 HC CDR2; Kabat): MISSGGSYTYYADSVKG
SEQ ID NO: 3 (MORAb-003 HC CDR3; Kabat): HGDDPAWFAY
SEQ ID NO: 4 (MORAb-003 LC CDR1; Kabat): SVSSSISSNNLH
SEQ ID NO: 5 (MORAb-003 LC CDR2: Kabat): GTSNLAS
SEQ ID NO: 6 (MORAb-003 LC CDR3; Kabat): QQWSSYPYMYT
SEQ ID NO: 7 (MORAb-003 HC CDR1; IMGT): GFTFSGYG
SEQ ID NO: 8 (MORAb-003 HC CDR2; IMGT): ISSGGSYT
SEQ ID NO: 9 (MORAb-003 HC CDR3; IMGT): ARHGDDPAWFAY
SEQ ID NO: 10 (MORAb-003 LC CDR1; IMGT): SSISSNN
SEQ ID NO: 11 (MORAb-003 LC CDR2; IMGT): GTS
SEQ ID NO: 12 (MORAb-003 LC CDR3; IMGT): QQWSSYPYMYT
SEQ ID NO: 15 (MORAb-003 Heavy chain (HC))
      1 EVQLVESGGG VVQPGRSLRL SCSASGFTFS GYGLSWVRQA PGKGLEWVAM
    51 ISSGGSYTYY ADSVKGRFAI SRDNAKNTLF LQMDSLRPED TGVYFCARHG
  101 DDPAWFAYWG QGTPVTVSSA STKGPSVEPL APSSKSTSGG TAALGCLVKD
  151 YFPEPVTVSW NSGALTSGVH TFPAVLQSSG LYSLSSVVTV PSSSLGTQTY
  201 ICNVNHKPSN TKVDKKVEPK SCDKTHTCPP CPAPELLGGP SVFLFPPKPK
  251 DTLMISRTPE VTCVVVDVSH EDPEVKENWY VDGVEVHNAK TKPREEQYNS
  301 TYRVVSVLTV LHQDWLNGKE YKCKVSNKAL PAPIEKTISK AKGQPREPQV
  351 YTLPPSRDEL TKNQVSLTCL VKGFYPSDIA VEWESNGQPE NNYKTTPPVL
  401 DSDGSFFLYS KLTVDKSRWQ QGNVFSCSVM HEALHNHYTQ KSLSLSPGK
SEQ ID NO: 16 (MORAb-003 Light chain (LC))
      1 DIQLTQSPSS LSASVGDRVT ITCSVSSSIS SNNLHWYQQK PGKAPKPWIY
    51 GTSNLASGVP SRFSGSGSGT DYTFTISSLQ PEDIATYYCQ QWSSYPYMYT
  101 FGQGTKVEIK RTVAAPSVFI FPPSDEQLKS GTASVVCLLN NFYPREAKVQ
  151 WKVDNALQSG NSQESVTEQD SKDSTYSLSS TLTLSKADYE KHKVYACEVT
  201 HQGLSSPVTK SENRGEC
SEQ ID NO: 33 (MORAb-003 Heavy Chain full length pre-protein
amino acid sequence; leader sequence underlined)
      1 MGWSCIILFL VATATGVHSE VQLVESGGGV VQPGRSLRLS CSASGFTFSG
    51 YGLSWVRQAP GKGLEWVAMI SSGGSYTYYA DSVKGRFAIS RDNAKNTLFL
  101 QMDSLRPEDT GVYFCARHGD DPAWFAYWGQ GTPVTVSSAS TKGPSVFPLA
  151 PSSKSTSGGT AALGCLVKDY FPEPVTVSWN SGALTSGVHT FPAVLQSSGL
  201 YSLSSVVTVP SSSLGTQTYI CNVNHKPSNT KVDKKVEPKS CDKTHTCPPC
  251 PAPELLGGPS VFLFPPKPKD TLMISRTPEV TCVVVDVSHE DPEVKENWYV
  301 DGVEVHNAKT KPREEQYNST YRVVSVLTVL HQDWLNGKEY KCKVSNKALP
  351 APIEKTISKA KGQPREPQVY TLPPSRDELT KNQVSLTCLV KGFYPSDIAV
  401 EWESNGQPEN NYKTTPPVLD SDGSFFLYSK LTVDKSRWQQ GNVFSCSVMH
  451 EALHNHYTQK SLSLSPGK
SEQ ID NO: 34 (MORAb-003 Light Chain full length pre-protein
amino acid sequence (leader sequence underlined))
      1 MGWSCIILFL VATATGVHSD IQLTQSPSSL SASVGDRVTI TCSVSSSISS
    51 NNLHWYQQKP GKAPKPWIYG TSNLASGVPS RFSGSGSGTD YTFTISSLQP
  101 EDIATYYCQQ WSSYPYMYTF GQGTKVEIKR TVAAPSVFIF PPSDEQLKSG
  151 TASVVCLLNN FYPREAKVQW KVDNALQSGN SQESVTEQDS KDSTYSLSST
  201 LTLSKADYEK HKVYACEVTH QGLSSPVTKS FNRGEC
SEQ ID NO: 35 (MORAb-003 HC nt)
      1 ATGGGATGGA GCTGTATCAT CCTCTTCTTG GTAGCAACAG CTACAGGTGT
    51 CCACTCCGAG GTCCAACTGG TGGAGAGCGG TGGAGGTGTT GTGCAACCTG
  101 GCCGGTCCCT GCGCCTGTCC TGCTCCGCAT CTGGCTTCAC CTTCAGCGGC
  151 TATGGGTTGT CTTGGGTGAG ACAGGCACCT GGAAAAGGTC TTGAGTGGGT
  201 TGCAATGATT AGTAGTGGTG GTAGTTATAC CTACTATGCA GACAGTGTGA
  251 AGGGTAGATT TGCAATATCG CGAGACAACG CCAAGAACAC ATTGTTCCTG
  301 CAAATGGACA GCCTGAGACC CGAAGACACC GGGGTCTATT TTTGTGCAAG
  351 ACATGGGGAC GATCCCGCCT GGTTCGCTTA TTGGGGCCAA GGGACCCCGG
  401 TCACCGTCTC CTCAGCCTCC ACCAAGGGCC CATCGGTCTT CCCCCTGGCA
  451 CCCTCCTCCA AGAGCACCTC TGGGGGCACA GCGGCCCTGG GCTGCCTGGT
  501 CAAGGACTAC TTCCCCGAAC CGGTGACGGT GTCGTGGAAC TCAGGCGCCC
  551 TGACCAGCGG CGTGCACACC TTCCCGGCTG TCCTACAGTC CTCAGGACTC
  601 TACTCCCTCA GCAGCGTGGT GACCGTGCCC TCCAGCAGCT TGGGCACCCA
  651 GACCTACATC TGCAACGTGA ATCACAAGCC CAGCAACACC AAGGTGGACA
  701 AGAAAGTTGA GCCCAAATCT TGTGACAAAA CTCACACATG CCCACCGTGC
  751 CCAGCACCTG AACTCCTGGG GGGACCGTCA GTCTTCCTCT TCCCCCCAAA
  801 ACCCAAGGAC ACCCTCATGA TCTCCCGGAC CCCTGAGGTC ACATGCGTGG
  851 TGGTGGACGT GAGCCACGAA GACCCTGAGG TCAAGTTCAA CTGGTACGTG
  901 GACGGCGTGG AGGTGCATAA TGCCAAGACA AAGCCGCGGG AGGAGCAGTA
  951 CAACAGCACG TACCGTGTGG TCAGCGTCCT CACCGTCCTG CACCAGGACT
1001 GGCTGAATGG CAAGGAGTAC AAGTGCAAGG TCTCCAACAA AGCCCTCCCA
1051 GCCCCCATCG AGAAAACCAT CTCCAAAGCC AAAGGGCAGC CCCGAGAACC
1101 ACAGGTGTAC ACCCTGCCCC CATCCCGGGA TGAGCTGACC AAGAACCAGG
1151 TCAGCCTGAC CTGCCTGGTC AAAGGCTTCT ATCCCAGCGA CATCGCCGTG
1201 GAGTGGGAGA GCAATGGGCA GCCGGAGAAC AACTACAAGA CCACGCCTCC
1251 CGTGCTGGAC TCCGACGGCT CCTTCTTCTT ATATTCAAAG CTCACCGTGG
1301 ACAAGAGCAG GTGGCAGCAG GGGAACGTCT TCTCATGCTC CGTGATGCAT
1351 GAGGCTCTGC ACAACCACTA CACGCAGAAG AGCCTCTCCC TGTCTCCCGG
1401 GAAATGA
SEQ ID NO: 36 (MORAb-003 LC nt)
      1 ATGGGATGGA GCTGTATCAT CCTCTTCTTG GTAGCAACAG CTACAGGTGT
    51 CCACTCCGAC ATCCAGCTGA CCCAGAGCCC AAGCAGCCTG AGCGCCAGCG
  101 TGGGTGACAG AGTGACCATC ACCTGTAGTG TCAGCTCAAG TATAAGTTCC
  151 AACAACTTGC ACTGGTACCA GCAGAAGCCA GGTAAGGCTC CAAAGCCATG
  201 GATCTACGGC ACATCCAACC TGGCTTCTGG TGTGCCAAGC AGATTCAGCG
  251 GTAGCGGTAG CGGTACCGAC TACACCTTCA CCATCAGCAG CCTCCAGCCA
  301 GAGGACATCG CCACCTACTA CTGCCAACAG TGGAGTAGTT ACCCGTACAT
  351 GTACACGTTC GGCCAAGGGA CCAAGGTGGA AATCAAACGA ACTGTGGCTG
  401 CACCATCTGT CTTCATCTTC CCGCCATCTG ATGAGCAGTT GAAATCTGGA
  451 ACTGCCTCTG TTGTGTGCCT GCTGAATAAC TTCTATCCCA GAGAGGCCAA
  501 AGTACAGTGG AAGGTGGATA ACGCCCTCCA ATCGGGTAAC TCCCAGGAGA
  551 GTGTCACAGA GCAGGACAGC AAGGACAGCA CCTACAGCCT CAGCAGCACC
  501 CTGACGCTGA GCAAAGCAGA CTACGAGAAA CACAAAGTCT ACGCCTGCGA
  651 AGTCACCCAT CAGGGCCTGA GCTCGCCCGT CACAAAGAGC TTCAACAGGG
  701 GAGAGTGTTA A
SEQ ID NO: 37 (human FRA)
      1 maqrmttqll lllvwvavvg eaqtriawar tellnvcmna khhkekpgpe dklheqcrpw
    61 rknaccstnt sqeahkdvsy lyrfnwnhcg emapackrhf iqdtclyecs pnlgpwiqqv
  121 dqswrkervl nvplckedce qwwedertsy tcksnwhkgw nwtsgfnkca vgaacqpfhf
  181 yfptptvlcn eiwthsykvs nysrgsgrci qmwfdpaqgn pneevarfya aamsgagpwa
  241 awpfllslal mllwlls
SEQ ID NO: 38 (human FRA nucleotide)
      1 cattccttgg tgccactgac cacagctctt tcttcaggga cagacatggc tcagcggatg
    61 acaacacagc tgctgctcct tctagtgtgg gtggctgtag taggggaggc tcagacaagg
  121 attgcatggg ccaggactga gcttctcaat gtctgcatga acgccaagca ccacaaggaa
  181 aagccaggcc ccgaggacaa gttgcatgag cagtgtcgac cctggaggaa gaatgcctgc
  241 tgttctacca acaccagcca ggaagcccat aaggatgttt cctacctata tagattcaac
  301 tggaaccact gtggagagat ggcacctgcc tgcaaacggc atttcatcca ggacacctgc
  361 ctctacgagt gctcccccaa cttggggccc tggatccagc aggtggatca gagctggcgc
  421 aaagagcggg tactgaacgt gcccctgtgc aaagaggact gtgagcaatg gtgggaagat
  481 tgtcgcacct cctacacctg caagagcaac tggcacaagg gctggaactg gacttcaggg
  541 tttaacaagt gcgcagtggg agctgcctgc caacctttcc atttctactt ccccacaccc
  601 actgttctgt gcaatgaaat ctggactcac tcctacaagg tcagcaacta cagccgaggg
  661 agtggccgct gcatccagat gtggttcgac ccagcccagg gcaaccccaa tgaggaggtg
  721 gcgaggttct atgctgcagc catgagtggg gctgggccct gggcagcctg gcctttcctg
  781 cttagcctgg ccctaatgct gctgtggctg ctcagctgac ctccttttac cttctgatac
  841 ctggaaatcc ctgccctgtt cagccccaca gctcccaact atttggttcc tgctccatgg
  901 tcgggcctct gacagccact ttgaataaac cagacaccgc acatgtgtct tgagaattat
  961 ttggaaaaaa aaaaaaaaaa aa
```

1. A method of treating a folate receptor alpha (FRA)-expressing cancer, comprising administering to a subject in need thereof an antibody-drug conjugate of Formula (I):
Ab-(L-D)p (I)
wherein
(i) Ab is an internalizing anti-folate receptor alpha antibody or internalizing antigen-binding fragment thereof comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 4 (LCDR1), SEQ ID NO: 5 (LCDR2), and SEQ ID NO: 6 (LCDR3), as defined by the Kabat numbering system; or three heavy chain complementarity determining regions (HCDRs) comprising amino acid sequences of SEQ ID NO: 7 (HCDR1), SEQ ID NO: 8 (HCDR2), and SEQ ID NO: 9 (HCDR3); and three light chain complementarity determining regions (LCDRs) comprising amino acid sequences of SEQ ID NO: 10 (LCDR1), SEQ ID NO: 11 (LCDR2), and SEQ ID NO: 12 (LCDR3), as defined by the IMGT numbering system;
(ii) D is eribulin;
(iii) L is a cleavable linker comprising Mal-(PEG)₂-Val-Cit-pAB; and
(iv) p is an integer from 1 to 8;
and wherein the antibody-drug conjugate is administered to said subject at a dose of 8 mg to 50 mg of the antibody-drug conjugate per square meter (m²) of the subject's body surface area (BSA).

2. A method of reducing risk of interstitial lung disease (ILD) in a subject being treated for an FRA-expressing cancer, comprising administering to the subject an antibody-drug conjugate of Formula (I):
Ab-(L-D)p (I)
wherein
(i) Ab is an internalizing anti-folate receptor alpha antibody or internalizing antigen-binding fragment thereof comprising three heavy chain complementarity determining regions (HCDRs) comprising amino acid sequences of SEQ ID NO: 1 (HCDR1), SEQ ID NO: 2 (HCDR2), and SEQ ID NO: 3 (HCDR3); and three light chain complementarity determining regions (LCDRs) comprising amino acid sequences of SEQ ID NO: 4 (LCDR1), SEQ ID NO: 5 (LCDR2), and SEQ ID NO: 6 (LCDR3), as defined by the Kabat numbering system; or three heavy chain complementarity determining regions (HCDRs) comprising amino acid sequences of SEQ ID NO: 7 (HCDR1), SEQ ID NO: 8 (HCDR2), and SEQ ID NO: 9 (HCDR3); and three light chain complementarity determining regions (LCDRs) comprising amino acid sequences of SEQ ID NO: 10 (LCDR1), SEQ ID NO: 11 (LCDR2), and SEQ ID NO: 12 (LCDR3), as defined by the IMGT numbering system;
(ii) D is eribulin;
(iii) L is a cleavable linker comprising Mal-(PEG)₂-Val-Cit-pAB; and
(iv) p is an integer from 1 to 8;
and wherein the antibody-drug conjugate is administered to said subject at a dose of 8 mg to 50 mg of the antibody-drug conjugate per square meter (m²) of the subject's body surface area (BSA).

3. The method of claim 1 or claim 2, wherein the antibody or antigen-binding fragment comprises a heavy chain variable region comprising an amino acid sequence of SEQ ID NO: 13, and a light chain variable region comprising an amino acid sequence of SEQ ID NO: 14.

4. The method of any one of claims 1 to 3, wherein the antibody or antigen-binding fragment comprises a heavy chain comprising an amino acid sequence of SEQ ID NO: 15, and a light chain comprising an amino acid sequence of SEQ ID NO: 16.

5. The method of any one of claims 1 to 4, wherein the antibody-drug conjugate is MORAb-202.

6. The method of any one of claims 1 to 5, wherein p is from 3 to 5.

7. The method of any one of claims 1 to 6, wherein the dose is 8 mg to 44 mg per m² of the subject's BSA.

8. The method of any one of claims 1 to 7, wherein the dose is 33 mg per m² of the subject's BSA.

9. The method of any one of claims 1 to 7, wherein the dose is 25 mg per m² of the subject's BSA.

10. The method of any one of claims 1 to 7, wherein the dose is 17 mg per m² of the subject's BSA.

11. The method of any one of claims 1 to 7, wherein the dose is 15 mg per m² of the subject's BSA.

12. The method of any one of claims 1 to 7, wherein the dose is 8 mg to 10 mg per m² of the subject's BSA.

13. The method of claim 8 or claim 9, wherein the method further comprises administering a lower dose per m² of the subject's BSA to reduce toxicity.

14. The method of claim 13, wherein the lower dose is 17 mg, 15 mg, or 8 mg to 10 mg per m² of the subject's BSA.

15. The method of any one of claims 1 to 14, wherein the antibody-drug conjugate is administered once every three weeks.

16. The method of any one of claims 1 to 14, wherein the antibody-drug conjugate is administered once every two weeks.

17. The method of any one of claims 1 to 14, wherein the antibody-drug conjugate is administered once per week.

18. The method of any one of claims 1 to 17, wherein the subject has a body weight value that is in the upper quartile for weight.

19. The method of any one of claims 1 to 18, wherein a risk of ILD is reduced by at least 5%, at least 10%, at least 15%, at least 16%, at least 17%, at least 18%, at least 19%, or at least 20% following administration of the antibody-drug conjugate, as compared to a treatment in which the antibody-drug conjugate is administered on a body weight based dose, e.g., at a dose of 0.5 to 2 mg per kilogram of the subject's body weight (BW), e.g., at a dose of 0.9 mg to 1.2 mg per kilogram of BW.

20. The method of any one of claims 1 to 19, further comprising administering a corticosteroid.

21. The method of claim 20, wherein the corticosteroid is administered prophylactically.

22. The method of claim 20 or claim 21, wherein the corticosteroid is administered concurrently or sequentially with the antibody-drug conjugate.

23. The method of any one of claims 20 to 22, wherein the corticosteroid is administered before or after the antibody-drug conjugate is administered.

24. The method of any one of claims 20 to 23, wherein the corticosteroid is dexamethasone.

25. The method of claim 24, wherein the dexamethasone is administered at a dose of 4 mg dexamethasone.

26. The method of claim 24 or claim 25, wherein the dexamethasone is administered at least once a day.

27. The method of claim 26, wherein the dexamethasone is administered two times a day.

28. The method of claim 26 or claim 27, wherein the dexamethasone is administered for at least three days at the start of treatment with the antibody-drug conjugate.

29. The method of any one of claims 24 to 28, wherein the dexamethasone is administered orally.

30. The method of any one of claims 20 to 23, wherein the corticosteroid is prednisone.

31. The method of claim 30, wherein the prednisone is administered at a dose of 0.5 mg prednisone.

32. The method of claim 30, wherein the prednisone is administered at a dose of 1 mg prednisone.

33. The method of claim 30, wherein the prednisone is administered at a dose of 2 mg prednisone.

34. The method of any one of claims 30 to 33, wherein the prednisone is administered at least once a day.

35. The method of any one of claims 30 to 34, wherein the prednisone is administered for at least 14 days before the start of treatment with the antibody-drug conjugate.

36. The method of any one of claims 30 to 35, wherein the prednisone is administered orally.

37. The method of any one of claims 20 to 23, wherein the corticosteroid is methylprednisolone.

38. The method of claim 37, wherein the methylprednisolone is administered at a dose of 500-1000 mg prednisone.

39. The method of claim 37 or claim 38, wherein the methylprednisolone is administered at least once a day.

40. The method of any one of claims 37 to 39, wherein the methylprednisolone is administered for at least three days before the start of treatment with the antibody-drug conjugate.

41. The method of any one of claims 37 to 40, wherein the methylprednisolone is administered intravenously.

42. The method of any one of claims 1 to 41, wherein the antibody-drug conjugate is administered intravenously.

43. The method of any one of claims 1 to 42, wherein the subject is administered a dose of 33 mg/m² and subsequently administered a reduced dose of 25 mg/m².

44. The method of any one of claims 1 to 42, wherein the subject is administered a dose of 25 mg/m² and subsequently administered a reduced dose of 17 mg/m².

45. The method of any one of claims 1 to 42, wherein the subject is administered a dose of 25 mg/m² and subsequently administered a reduced dose of 15 mg/m².

46. The method of any one of claims 1 to 42, wherein the subject is administered a dose of 15 mg/m² and subsequently administered a reduced dose of 8 mg/m² to 10 mg/m².

47. The method of any one of claims 1 to 46, wherein the FRA-expressing cancer is selected from: gastric cancer, ovarian cancer, serous ovarian cancer, serous high-grade ovarian cancer, clear cell ovarian cancer, platinum resistant ovarian cancer, lung cancer, non-small cell lung cancer, metastatic non-small cell lung cancer, lung carcinoid, colorectal cancer, breast cancer, triple negative breast cancer, hormone receptor (HR)-positive and HER2-low breast cancer, endometrial cancer, serous endometrial carcinoma, peritoneal cancer, primary peritoneal cancer, fallopian tube cancer, pancreatic cancer, kidney cancer, renal cell cancer, cervical cancer, esophageal cancer, and osteosarcoma.

48. The method of claim 47, wherein the FRA-expressing cancer is ovarian cancer.

49. The method of claim 48, wherein the ovarian cancer is platinum resistant ovarian cancer (PROC).

50. The method of claim 49, wherein the PROC is a serous ovarian cancer.

51. The method of claim 50, wherein the serous ovarian cancer is a high-grade serous ovarian cancer.

52. The method of claim 47, wherein the FRA-expressing cancer is platinum resistant primary peritoneal cancer.

53. The method of claim 47, wherein the FRA-expressing cancer is platinum resistant fallopian tube cancer.

54. The method of claim 47, wherein the FRA-expressing cancer is breast cancer.

55. The method of claim 54, wherein the breast cancer is triple negative breast cancer (TNBC).

56. The method of claim 47, wherein the FRA-expressing cancer is non-small cell lung cancer (NSCLC).

57. The method of claim 47, wherein the FRA-expressing cancer is endometrial cancer (EC).

58. The method of any one of claims 47 to 57, wherein the FRA-expressing cancer is a metastatic cancer.

59. The method of claim 58, wherein the metastatic cancer has no genomic alteration.

60. The method of claim 58, wherein the metastatic cancer has at least one known genomic alteration in at least one of any of the following genes: EGFR, ALK, PI3K, AKT, mTOR, RET, MET, BRAF, NTRK, ROS1, and any gene involved in the RAS-MAPKpathway.

61. The method of any one of claims 58 to 60, wherein the metastatic cancer is a non-small cell lung cancer.

62. The method of any one of claims 47 to 61, wherein the FRA-expressing cancer is a refractory cancer.

63. The method of claim 62, wherein the refractory cancer is non-responsive to a targeted treatment.

64. The method of claim 63, wherein the targeted treatment is a targeted treatment against any one of the following genes or variants thereof: EGFR, ALK, BRAF, RET, MET, NTRK, and ROS1.

65. The method of claim 62, wherein the refractory cancer is non-responsive to a platinum-based treatment and an immunotherapy-based treatment, wherein the treatments are administered concurrently or sequentially.

66. The method of claim 65, wherein the platinum-based treatment is a platinum-doublet chemotherapy, and wherein the immunotherapy-based treatment is a PD-1 inhibitor or a PD-L1 inhibitor.

67. The method of any one of claims 1 to 66, wherein the subject at the start of treatment does not have one or more of: interstitial lung disease (ILD) and/or pneumonitis, a history of ILD and/or pneumonitis, a lung-specific clinically significant illness, pleural effusion, pericardial effusion, prior pneumonectomy, a history of chest radiotherapy within the past 2 years, autoimmune disorder with pulmonary involvement, connective tissue disorder with pulmonary involvement, or inflammatory disorder with pulmonary involvement.

68. The method of any one of claims 1 to 66, wherein the subject does not have one or more of: a history of more than three prior therapies for the FRA-expressing cancer, a high neutrophil-to-lymphocyte ratio, or a serum albumin level at the start of treatment of less than 3 g/dL.


SEQUENCE LISTING (38 sequences)

SEQ ID NO: 1 - protein, 5 aa, synthetic construct
GYGLS

SEQ ID NO: 2 - protein, 17 aa, synthetic construct
MISSGGSYTYYADSVKG

SEQ ID NO: 3 - protein, 10 aa, synthetic construct
HGDDPAWFAY

SEQ ID NO: 4 - protein, 12 aa, synthetic construct
SVSSSISSNNLH

SEQ ID NO: 5 - protein, 7 aa, synthetic construct
GTSNLAS

SEQ ID NO: 6 - protein, 11 aa, synthetic construct
QQWSSYPYMYT

SEQ ID NO: 7 - protein, 8 aa, synthetic construct
GFTFSGYG

SEQ ID NO: 8 - protein, 8 aa, synthetic construct
ISSGGSYT

SEQ ID NO: 9 - protein, 12 aa, synthetic construct
ARHGDDPAWFAY

SEQ ID NO: 10 - protein, 7 aa, synthetic construct
SSISSNN

SEQ ID NO: 11 - skipped

SEQ ID NO: 12 - protein, 11 aa, synthetic construct
QQWSSYPYMYT

SEQ ID NO: 13 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 14 - protein, 110 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 15 - protein, 449 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 217 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 15 bp, synthetic construct
  source 1..15: mol_type = other DNA
ggctatgggttgtct

SEQ ID NO: 18 - DNA, 51 bp, synthetic construct
  source 1..51: mol_type = other DNA
atgattagtagtggtggtagttatacctactatgcagacagtgtgaagggt

SEQ ID NO: 19 - DNA, 30 bp, synthetic construct
  source 1..30: mol_type = other DNA
catggggacgatcccgcctggttcgcttat

SEQ ID NO: 20 - DNA, 36 bp, synthetic construct
  source 1..36: mol_type = other DNA
agtgtcagctcaagtataagttccaacaacttgcac

SEQ ID NO: 21 - DNA, 21 bp, synthetic construct
  source 1..21: mol_type = other DNA
ggcacatccaacctggcttct

SEQ ID NO: 22 - DNA, 33 bp, synthetic construct
  source 1..33: mol_type = other DNA
caacagtggagtagttacccgtacatgtacacg

SEQ ID NO: 23 - DNA, 24 bp, synthetic construct
  source 1..24: mol_type = other DNA
ggcttcaccttcagcggctatggg

SEQ ID NO: 24 - DNA, 24 bp, synthetic construct
  source 1..24: mol_type = other DNA
attagtagtggtggtagttatacc

SEQ ID NO: 25 - DNA, 36 bp, synthetic construct
  source 1..36: mol_type = other DNA
gcaagacatggggacgatcccgcctggttcgcttat

SEQ ID NO: 26 - DNA, 21 bp, synthetic construct
  source 1..21: mol_type = other DNA
tcaagtataagttccaacaac

SEQ ID NO: 27 - skipped

SEQ ID NO: 28 - DNA, 33 bp, synthetic construct; identical to SEQ ID NO: 22
  source 1..33: mol_type = other DNA

SEQ ID NO: 29 - DNA, 357 bp, synthetic construct
  source 1..357: mol_type = other DNA
gaggtccaactggtggagagcggtggaggtgttgtgcaacctggccggtccctgcgcctgtcctgctccgcatctggcttcaccttcagcggctatgggttgtcttgggtgagacaggcacctggaaaaggtcttgagtgggttgcaatgattagtagtggtggtagttatacctactatgcagacagtgtgaagggtagatttgcaatatcgcgagacaacgccaagaacacattgttcctgcaaatggacagcctgagacccgaagacaccggggtctatttttgtgcaagacatggggacgatcccgcctggttcgcttattggggccaagggaccccggtcaccgtctcctca

SEQ ID NO: 30 - DNA, 330 bp, synthetic construct
  source 1..330: mol_type = other DNA
gacatccagctgacccagagcccaagcagcctgagcgccagcgtgggtgacagagtgaccatcacctgtagtgtcagctcaagtataagttccaacaacttgcactggtaccagcagaagccaggtaaggctccaaagccatggatctacggcacatccaacctggcttctggtgtgccaagcagattcagcggtagcggtagcggtaccgactacaccttcaccatcagcagcctccagccagaggacatcgccacctactactgccaacagtggagtagttacccgtacatgtacacgttcggccaagggaccaaggtggaaatcaaa

SEQ ID NO: 31 - DNA, 1350 bp, synthetic construct
  source 1..1350: mol_type = other DNA
gaggtccaactggtggagagcggtggaggtgttgtgcaacctggccggtccctgcgcctgtcctgctccgcatctggcttcaccttcagcggctatgggttgtcttgggtgagacaggcacctggaaaaggtcttgagtgggttgcaatgattagtagtggtggtagttatacctactatgcagacagtgtgaagggtagatttgcaatatcgcgagacaacgccaagaacacattgttcctgcaaatggacagcctgagacccgaagacaccggggtctatttttgtgcaagacatggggacgatcccgcctggttcgcttattggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 32 - DNA, 654 bp, synthetic construct
  source 1..654: mol_type = other DNA
gacatccagctgacccagagcccaagcagcctgagcgccagcgtgggtgacagagtgaccatcacctgtagtgtcagctcaagtataagttccaacaacttgcactggtaccagcagaagccaggtaaggctccaaagccatggatctacggcacatccaacctggcttctggtgtgccaagcagattcagcggtagcggtagcggtaccgactacaccttcaccatcagcagcctccagccagaggacatcgccacctactactgccaacagtggagtagttacccgtacatgtacacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 33 - protein, 468 aa, synthetic construct
MGWSCIILFLVATATGVHSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 236 aa, synthetic construct
MGWSCIILFLVATATGVHSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 1407 bp, synthetic construct
  source 1..1407: mol_type = other DNA
atgggatggagctgtatcatcctcttcttggtagcaacagctacaggtgtccactccgaggtccaactggtggagagcggtggaggtgttgtgcaacctggccggtccctgcgcctgtcctgctccgcatctggcttcaccttcagcggctatgggttgtcttgggtgagacaggcacctggaaaaggtcttgagtgggttgcaatgattagtagtggtggtagttatacctactatgcagacagtgtgaagggtagatttgcaatatcgcgagacaacgccaagaacacattgttcctgcaaatggacagcctgagacccgaagacaccggggtctatttttgtgcaagacatggggacgatcccgcctggttcgcttattggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 36 - DNA, 711 bp, synthetic construct
  source 1..711: mol_type = other DNA
atgggatggagctgtatcatcctcttcttggtagcaacagctacaggtgtccactccgacatccagctgacccagagcccaagcagcctgagcgccagcgtgggtgacagagtgaccatcacctgtagtgtcagctcaagtataagttccaacaacttgcactggtaccagcagaagccaggtaaggctccaaagccatggatctacggcacatccaacctggcttctggtgtgccaagcagattcagcggtagcggtagcggtaccgactacaccttcaccatcagcagcctccagccagaggacatcgccacctactactgccaacagtggagtagttacccgtacatgtacacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 37 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 38 - DNA, 982 bp, Homo sapiens
  source 1..982: mol_type = unassigned DNA
cattccttggtgccactgaccacagctctttcttcagggacagacatggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctgacctccttttaccttctgatacctggaaatccctgccctgttcagccccacagctcccaactatttggttcctgctccatggtcgggcctctgacagccactttgaataaaccagacaccgcacatgtgtcttgagaattatttggaaaaaaaaaaaaaaaaaa